(19) United States
(12) Patent Application Publication
(10) Pub. No.: US 2010/0298348 A1
(43) Pub. Date: Nov. 25, 2010

(54) Method of Decreasing Ubiquitylated Protein Levels

(76) Inventors: Kim Nicholas Green, Newport Beach, CA (US); Eckard Weber, San Diego, CA (US); Tilman Oltersdorf, Cardiff, CA (US); Sharon Rogers, St. George, UT (US); Frank LaFerla, Irvine, CA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/777,586

(22) Filed: May 11, 2010

Related U.S. Application Data
(60) Provisional application No. 61/177,234, filed on May 11, 2009.
(60) Provisional application No. 61/177,235, filed on May 11, 2009.
(60) Provisional application No. 61/177,236, filed on May 11, 2009.
(60) Provisional application No. 61/177,237, filed on May 11, 2009.

(51) Int. Cl. A61K 31/519; A61K 31/438; A61K 31/437; A61K 31/429; A61K 31/4166; A61P 9/00; A61P 43/00; A61P 25/28; A61P 29/00; A61P 25/16; A61P 25/00; A61P 35/00; A61P 19/08; A61P 3/10; A61P 27/12; A61P 27/00; C12Q 1/02

(52) U.S. Cl. 514/259.1; 514/278; 514/292; 514/300; 514/368; 514/392; 435/29

(57) ABSTRACT

The present invention provides a method of decreasing the level of ubiquitylated protein in a subject, the method comprising administering a heterocyclic compound disclosed herein or a pharmaceutically acceptable salt, hydrate or prodrug thereof to a subject in need thereof.

This application claims priority benefit of U.S. Provisional Application Nos. 61/177,234, 61/177,235, 61/177,236 and 61/177,237, each of which was filed on May 11, 2009, and each of which is incorporated by reference in its entirety.

### FIELD OF THE INVENTION

This invention relates to the processing of proteins that are targeted for degradation by ubiquitylation.

### BACKGROUND OF THE INVENTION

Alzheimer's Disease (AD) is a neurodegenerative disorder for which there are only symptomatic treatments, with limited efficacy. Certain amyloid-beta (Aβ) fragments of APP, notably Aβ₁₋₄₀ and Aβ₁₋₄₂ have been implicated in the pathology of AD. Reduction of Aβ has been pursued as an approach to modify the course of AD (Barten, D. and C. Albright, Mol. Neurobiol. 37: 171-186 (1998)). However, to date, no approved therapies have resulted from this approach.

Attempts have been made to treat AD with both active and passive immunization against Aβ. One such immunization approach has already failed in human testing (Holmes, C. et al., Lancet 372: 216-23 (2008)). A limitation of Aβ immunotherapy may be that it targets only Aβ that is already formed. It does not slow or halt production of new Aβ, and in fact, may even encourage increased production of new Aβ.

Other attempts to treat AD have involved interrupting known enzymes from the processing of APP before deleterious Aβ fragments can be produced. These enzyme targets are gamma-secretase and beta-secretase (BACE), a type 1 membrane-associated aspartyl protease. Gamma-secretase inhibitors have not proved useful, because many such inhibitors affect cleavage of other gamma-secretase substrates and as a result can be toxic (Czirr, E. and S. Weggen, Neurodegenerative Dis. 3: 298-302 (2006); Tomita, T., Nauyn-Schmiedegerg's Arch. Pharmacol. 377: 295-300 (2008)); Milano, J. et al., Toxicological Sciences 82: 341-358 (2004)).

Gamma-secretase modulators also have not proved useful. Examples of gamma secretase modulators include non-steroidal anti-inflammatory drugs (NSAIDs), which are allosteric modulators of gamma secretase. Such compounds are not toxic at the doses used for inflammation, but do have toxicity at doses high enough to modulate gamma secretase. The compounds that have entered clinical testing have only high micromolar in vitro potency, such that they are too weak to have sufficient clinical effects (Czirr, E. and S. Weggen, Neurodegenerative Dis. 3: 298-304 (2006)). The prototype gamma secretase modulator, Flurizan, recently failed a Phase III clinical trial.

In addition, prior attempts at treating AD with beta-secretase inhibitors have not proven useful, because the large binding pocket of beta-secretase combined with its membrane location creates a challenge to design inhibitors that cross the blood-brain barrier in sufficient concentration to be useful (Barten, D. and C. Albright, Mol. Neurobiol 37: 171-186 (1998); John, V., Curr. Top. Med. Chem. 6: 569-78 (2006); Venugopal, C. et al., CNS & Neurological Disorders—Drug Targets 7: 278-294 (2008)).

Thus, there remains a need in the art for an effective treatment of AD.

The putative alpha-secretase ADAM10 is a surface-expressed metalloproteinase that plays an important role in various physiological processes. ADAM10 is initially expressed as the inactive enzyme pro-ADAM10, which is subsequently activated by proteolytic cleavage to give the active enzyme ADAM 10. ADAM 10 is known to cleave substrates at extracellular sites proximal to the cellular membrane, resulting in release of the soluble ectodomain of the substrate. Mice with a targeted disruption of the adam10 gene have shown that the protease is crucial for development while recent in vitro research has shown that ADAM10 is involved in various diseases and repair functions. See Pruessmeyer, J. and Ludwig, A., Semin. Cell & Dev. Biol. 1-11 (2008).

The acute and chronic inflammatory response is driven by cytokines that, in turn, drive gene expression of proinflammatory molecules such as chemokines and adhesion molecules that are involved in leukocyte recruitment. One major proinflammatory cytokine tumor necrosis factor α (TNFα) has been found to be a substrate of ADAM10. Other proinflammatory substrates of ADAM10 include Notch, the IL-6 receptor, CX3CL1, CXCL16, JAM-A, VE-cadherin and Fas-ligand. See Pruessmeyer, J. and Ludwig, A., Semin. Cell & Dev. Biol. 1-11 (2008). Thus, downregulation of ADAM10 activity can lead to control of the inflammatory response.

Overexpression of ADAM10 has been associated with cancer, such as prostate cancer, colon carcinoma and squamous cell carcinoma. Previously, metalloproteinases had been associated with tumor invasion due to facilitating tumor cell access to the vascular and lymphatic system. Recently, ADAM10 has been indicated to be involved in early tumorigenesis events such as stimulation of proliferation by released growth factors or escape from immune surveillance. ADAM10 substrates known to be involved in tumorigenesis include EGF, betacellulin, ErbB2/HER2, CD44, Des 2, MICA and CD30. See Pruessmeyer, J. and Ludwig, A., Semin. Cell & Dev. Biol. 1-11 (2008). Thus, downregulation of ADAM10 can lead to control of early tumorigenesis by decreased shedding of growth factors, adhesion molecules and molecules which help cancer evade immune surveillance.

ADAM10 has also been shown to cleave a low affinity IgE (CD23) receptor. See Lemieux, G. A. et al., J. Biol. Chem. 282: 14,836-44 (2007); and Weskamp et al., Nature Immunol. 7: 1293-98 (2006). Thus, downregulation of ADAM10 can lead to control of allergic reaction.

ADAM10 has also been shown to be involved in mediating Gram-positive bacteria activation of mucin gene expression in cystic fibrosis patients, leading to overproduction of mucous, which contributes to morbidity and mortality by obstructing airflow and shielding bacteria from antibiotics. See Lemjabber, H. and C. Basbaum, Nature Medicine 8: 41-46 (2002). Thus, downregulation of ADAM10 can lead to control of mucous overproduction in cystic fibrosis patients.

Reduction of protein concentration can be due to reduced transcription, reduced translation, and/or increased degradation. The majority of cellular proteins are degraded by the ubiquitin-proteasome system (UPS) which consists of a substrate recruiting and substrate degrading machinery. Substrate recruiting is mediated by three classes of enzymes: E1 activates ubiquitin, E2 serves as a ubiquitin carrier and E3 is a ubiquitin protein ligase that attaches activated ubiquitin to protein substrates. Ubiquitylated proteins are recognized by the nature of the ubiquitin linkage and targeted to either lysosomes (Lys63 linkage) or to the proteasome (Lys11, Lys29 or Lys48 linkages) and degraded (for review see Dahlmann, BMC Biochem. 22: Suppl 1:S3 (2007) and Miranda et al., Molecular Interventions 7: 157-167 (2007)). The UPS degrades damaged proteins and is a key mechanism for removing dysfunctional proteins created by transcription, translation and/or folding errors. With increasing age the UPS becomes less efficient. Accumulation and subsequent aggregation of proteins that should be removed by the UPS are believed to play a role in a number of age-related diseases, including neurodegenerative diseases. In particular, it has been hypothesized that beta-amyloid accumulation in Alzheimer's disease, is, in part, a consequence of failure of the UPS and/or lysosomal processing pathways to clear Aβ aggregates. Pathological protein accumulation is involved in a number of other neurodegenerative diseases such as Parkinson's disease (α-synuclein Lewy bodies) and Huntington's Disease (huntingtin protein) (for review see Upadhya et al., BMC Biochem. 22: Suppl 1:S1 (2007)).

In addition, it has been shown that BACE degradation is mediated by the ubiquitin proteosome pathway. Qing, H. et al., FASEB J. 18:1571-1573 (2004).

### BRIEF SUMMARY OF THE INVENTION

The present invention provides a method of inducing cleavage of amyloid precursor protein to produce an approximately 17 kilodalton (kDa) carboxy-terminal fragment of amyloid precursor protein in a subject, the method comprising administering an effective amount of a heterocyclic compound having the general Formula (I):

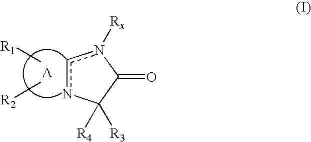

or a pharmaceutically acceptable salt, hydrate or prodrug thereof to a subject in need thereof, wherein the approximately 17 kDA fragment includes the carboxy-terminal amino acid sequence of amyloid precursor protein and amyloid-beta amino acid sequence, wherein R₁, R₂, R₃, R₃ and Rₓ are as defined herein.

The present invention also provides an approximately 17 kDa amyloid precursor protein fragment that includes the carboxy-terminal amino acid sequence of amyloid precursor protein and amyloid-beta amino acid sequence.

The present invention also provides a method for screening for a compound that cleaves amyloid precursor protein to generate an approximately 17 kDa fragment of amyloid precursor protein, the method comprising: (a) exposing cells that produce amyloid precursor protein or fragments thereof to a test compound, and (b) detecting the amount of the approximately 17 kDa fragment, wherein the approximately 17 kDa fragment includes the carboxy-terminal amino acid sequence of amyloid precursor protein and amyloid-beta amino acid sequence, and wherein an increase in the amount of the approximately 17 kDa fragment in cells that are exposed to the compound, relative to the amount of the approximately 17 kDa fragment in cells that are not exposed to the compound, indicates that the compound cleaves amyloid precursor protein to generate the approximately 17 kDa fragment.

The present invention also provides a method of inducing cleavage of amyloid precursor protein to produce an approximately 17 kDa carboxy-terminal fragment of amyloid precursor protein in a subject, the method comprising administering an effective amount of a compound that is not a compound having the general Formula (I):

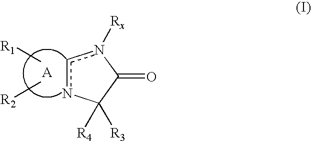

or a pharmaceutically acceptable salt, hydrate or prodrug thereof, wherein R₁, R₂, R₃, R₃ and Rₓ are as defined herein.

The present invention also provides a method of decreasing the level of pro-ADAM10 and/or BACE protein in a subject, the method comprising administering an effective amount of a heterocyclic compound having the general Formula (I):

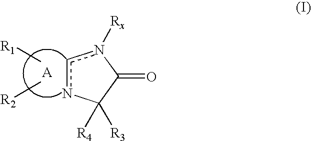

or a pharmaceutically acceptable salt, hydrate or prodrug thereof to a subject in need thereof, wherein R₁, R₂, R₃, R₃ and Rₓ are as defined herein.

The present invention also provides a method for screening for a compound that decreases the level of pro-ADAM10 and/or BACE, the method comprising: (a) exposing cells or tissue that express pro-ADAM10 and/or BACE to a test compound, and (b) detecting the amount of pro-ADAM10 and/or BACE in the cells or tissue, wherein a decrease in the amount pro-ADAM10 and/or BACE protein in cells or tissue exposed to the compound, relative to pro-ADAM10 and/or BACE protein in cells or tissue that are not exposed to the compound, indicates that the compound decreased the amount of pro-ADAM10 and/or BACE protein.

The present invention also provides a method of decreasing the level of pro-ADAM10 and/or BACE protein in a subject, the method comprising administering an effective amount of a heterocyclic compound that is not a compound having the general Formula (I):

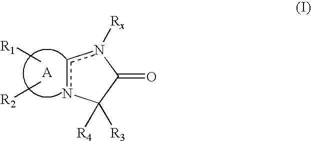

or a pharmaceutically acceptable salt, hydrate or prodrug thereof to a subject in need thereof, wherein R₁, R₂, R₃, R₃ and Rₓ are as defined herein.

The present invention also provides an isolated approximately 32 kDa phosphorylated tau protein fragment.

The present invention also provides a method of decreasing tau protein accumulation in a subject, the method comprising administering an effective amount of a heterocyclic compound having the general Formula (I):

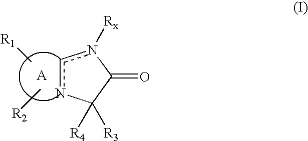

or a pharmaceutically acceptable salt, hydrate or prodrug thereof to a subject in need thereof, wherein R₁, R₂, R₃, R₃ and Rₓ are as defined herein.

The present invention also provides a method for screening for a compound that decreases tau protein accumulation, the method comprising: (a) exposing cells or tissue that accumulate tau protein to a test compound, and (b) detecting the amount of tau protein accumulated in said cells or tissue, wherein a decrease in the amount of tau protein accumulation in cells exposed to the compound, relative to tau protein accumulation by cells or tissue that are not exposed to the compound, indicates that the compound decreased the amount of tau protein accumulation.

The present invention also provides a method of decreasing tau protein accumulation in a subject, the method comprising administering an effective amount of a heterocyclic compound that is not a compound having the general Formula (I):

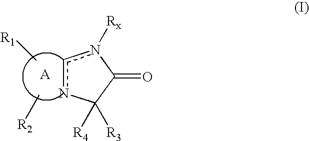

or a pharmaceutically acceptable salt, hydrate or prodrug thereof to a subject in need thereof, wherein R₁, R₂, R₃, R₃ and Rₓ are as defined herein, and wherein the compound is not a compound disclosed in International Application No. PCT/US2006/026331, which published as WO 2007/008586.

The present invention also provides a method of treating or preventing inflammation in a subject, the method comprising administering an effective amount of a heterocyclic compound having the general Formula (I):

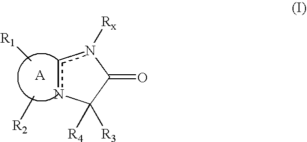

or a pharmaceutically acceptable salt, hydrate or prodrug thereof to a subject in need thereof, wherein R₁, R₂, R₃, R₃ and Rₓ are as defined herein.

The present invention also provides a method of treating or preventing cystic fibrosis in a subject, the method comprising administering an effective amount of a heterocyclic compound having the general Formula (I):

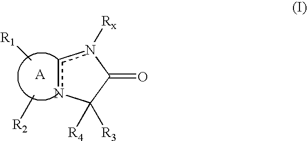

or a pharmaceutically acceptable salt, hydrate or prodrug thereof to a subject in need thereof, wherein R₁, R₂, R₃, R₃ and Rₓ are as defined herein.

The present invention also provides a method of treating or preventing allergy in a subject, the method comprising administering an effective amount of a heterocyclic compound having the general Formula (I):

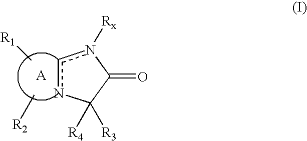

or a pharmaceutically acceptable salt, hydrate or prodrug thereof to a subject in need thereof, wherein R₁, R₂, R₃, R₃ and Rₓ are as defined herein.

The present invention also provides a method of treating hyperproliferative disease in a subject, the method comprising administering an effective amount of a heterocyclic compound having the general Formula (I):

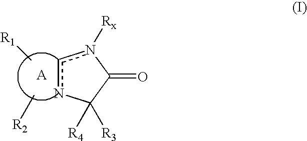

or a pharmaceutically acceptable salt, hydrate or prodrug thereof to a subject in need thereof, wherein R₁, R₂, R₃, R₃ and Rₓ are as defined herein.

The present invention also provides a method of reducing levels of ubiquitylated proteins in a human subject, the method comprising administering an effective amount of a heterocyclic compound having the general Formula (I):

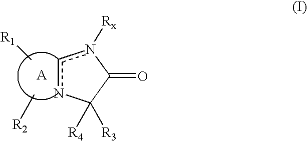

or a pharmaceutically acceptable salt, hydrate or prodrug thereof to a human subject in need thereof, wherein R₁, R₂, R₃, R₃ and Rₓ are as defined herein.

The present invention also provides a method of reducing levels of ubiquitylated proteins in a human subject, the method comprising administering an effective amount of a compound that is not a heterocyclic compound having the general Formula (I):

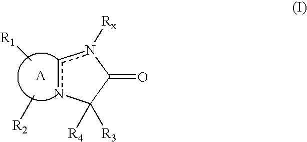

or a pharmaceutically acceptable salt, hydrate or prodrug thereof to a human subject in need thereof, wherein R₁, R₂, R₃, R₃ and Rₓ are as defined herein, and wherein the compound is not a ubiquitin hydrolase.

The present invention also provides a method of enhancing the removal and degradation of unwanted proteins in a human subject, the method comprising administering an effective amount of a heterocyclic compound having the general Formula (I):

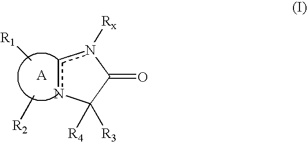

or a pharmaceutically acceptable salt, hydrate or prodrug thereof to a human subject in need thereof, wherein R₁, R₂, R₃, R₃ and Rₓ are as defined herein.

The present invention also provides a method of enhancing the removal and degradation of harmful proteins in a human subject, the method comprising administering an effective amount of a compound that is not a heterocyclic compound having the general Formula (I):

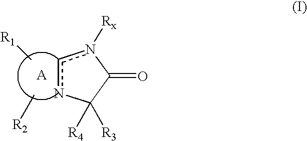

or a pharmaceutically acceptable salt, hydrate or prodrug thereof to a human subject in need thereof, wherein R₁, R₂, R₃, R₃ and Rₓ are as defined herein, and wherein the compound is not a ubiquitin hydrolase.

The present invention also provides a method of enhancing proteasome breakdown of accumulated and/or harmful proteins in a human subject, the method comprising administering an effective amount of a heterocyclic compound having the general Formula (I):

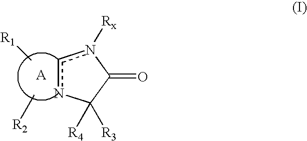

or a pharmaceutically acceptable salt, hydrate or prodrug thereof to a human subject in need thereof, wherein R₁, R₂, R₃, R₃ and Rₓ are as defined herein.

The present invention also provides a method of enhancing proteasome breakdown of accumulated and/or harmful proteins in a human subject, the method comprising administering an effective amount of a compound that is not a heterocyclic compound having the general Formula (I):

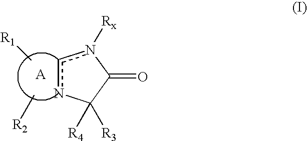

or a pharmaceutically acceptable salt, hydrate or prodrug thereof to a human subject in need thereof, wherein R₁, R₂, R₃, R₃ and Rₓ are as defined herein, and wherein the compound is not a ubiquitin hydrolase.

The present invention also provides a method of enhancing the activity of the ubiquitin-proteasome system pathway in a human subject, the method comprising administering an effective amount of a heterocyclic compound having the general Formula (I):

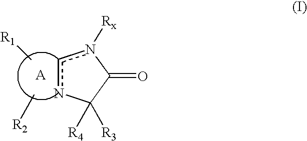

or a pharmaceutically acceptable salt, hydrate or prodrug thereof to a human subject in need thereof, wherein R₁, R₂, R₃, R₃ and Rₓ are as defined herein.

The present invention also provides a method of enhancing the activity of the ubiquitin-proteasome system pathway in a human subject, the method comprising administering an effective amount of a compound that is not a heterocyclic compound having the general Formula (I):

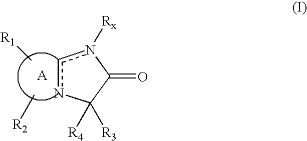

or a pharmaceutically acceptable salt, hydrate or prodrug thereof to a human subject in need thereof, wherein R₁, R₂, R₃, R₃ and Rₓ are as defined herein, and wherein the compound is not a ubiquitin hydrolase.

The present invention also provides a method for screening for a compound that decreases the level of ubiquitylated protein, the method comprising: (a) exposing cells or tissue that contain ubiquitylated protein to a test compound, and (b) detecting the amount of ubiquitylated protein in said cells or tissue, wherein an decrease in the amount ubiquitylated protein in cells or tissue exposed to the compound, relative to ubiquitylated protein in cells or tissue that are not exposed to the compound, indicates that the compound decreased the amount of ubiquitylated protein.

The present invention also provides a method of treating or preventing a prion disease, the method comprising administering an effective amount of a heterocyclic compound having the general Formula (I):

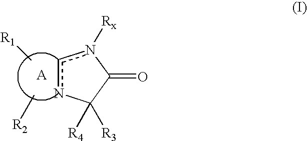

or a pharmaceutically acceptable salt, hydrate or prodrug thereof to a human subject in need thereof, wherein R₁, R₂, R₃, R₃ and Rₓ are as defined herein.

The present invention also provides a method of treating or preventing cataracts of the eye, the method comprising administering an effective amount of a heterocyclic compound having the general Formula (I):

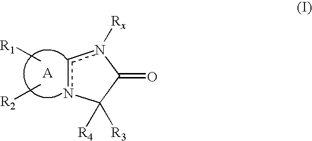

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has cataracts of the eye. In another embodiment, the subject has been diagnosed with cataracts of the eye. In another embodiment, the cataracts of the eye are treated. In another embodiment, the cataracts of the eye are prevented.

The present invention also provides a method of treating or preventing type 2 diabetes, the method comprising administering an effective amount of a heterocyclic compound having the general Formula (I):

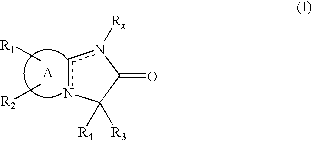

or a pharmaceutically acceptable salt, hydrate or prodrug thereof to a human subject in need thereof, wherein R₁, R₂, R₃, R₃ and Rₓ are as defined herein.

The present invention also provides a method of treating or preventing Paget's disease, the method comprising administering an effective amount of a heterocyclic compound having the general Formula (I):

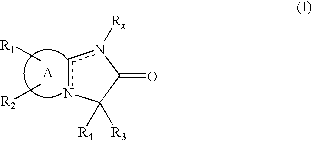

or a pharmaceutically acceptable salt, hydrate or prodrug thereof to a human subject in need thereof, wherein R₁, R₂, R₃, R₃ and Rₓ are as defined herein.

The present invention also provides a method of treating traumatic brain injury, the method comprising administering an effective amount of a heterocyclic compound having the general Formula (I):

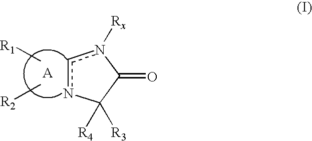

or a pharmaceutically acceptable salt, hydrate or prodrug thereof to a human subject in need thereof, wherein R₁, R₂, R₃, R₃ and Rₓ are as defined herein.

The present invention also provides a method of treating or preventing Amyotrophic Lateral Sclerosis, the method comprising administering an effective amount of a heterocyclic compound having the general Formula (I):

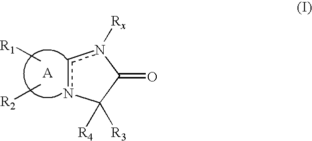

or a pharmaceutically acceptable salt, hydrate or prodrug thereof to a human subject in need thereof, wherein R₁, R₂, R₃, R₃ and Rₓ are as defined herein.

The present invention also provides a method of treating or preventing frontotemporolobar dementia, the method comprising administering an effective amount of a heterocyclic compound having the general Formula (I):

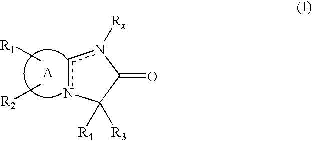

or a pharmaceutically acceptable salt, hydrate or prodrug thereof to a human subject in need thereof, wherein R₁, R₂, R₃, R₃ and Rₓ are as defined herein.

The present invention also provides a method of treating or preventing Lewy body disease, the method comprising administering an effective amount of a heterocyclic compound having the general Formula (I):

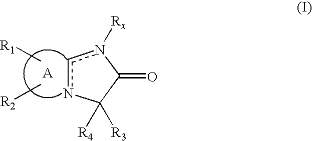

or a pharmaceutically acceptable salt, hydrate or prodrug thereof to a human subject in need thereof, wherein R₁, R₂, R₃, R₃ and Rₓ are as defined herein.

The present invention also provides a method of treating or preventing sporadic inclusion body myositis, the method comprising administering an effective amount of a heterocyclic compound having the general Formula (I):

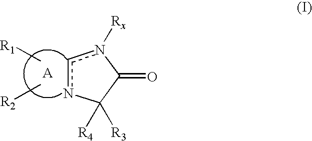

or a pharmaceutically acceptable salt, hydrate or prodrug thereof to a human subject in need thereof, wherein R₁, R₂, R₃, R₃ and Rₓ are as defined herein.

The present invention also provides a method of treating or preventing cardiac dysfunction, the method comprising administering a heterocyclic compound having the general Formula (I):

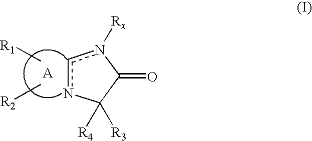

or a pharmaceutically acceptable salt, hydrate or prodrug thereof to a human subject in need thereof, wherein R₁, R₂, R₃, R₃ and Rₓ are as defined herein.

The present invention also provides a method of treating or preventing spinobulbar muscular atrophy (Kennedy Disease), the method comprising administering an effective amount of a heterocyclic compound having the general Formula (I):

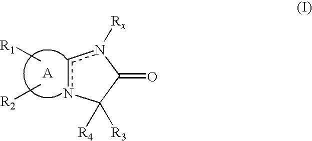

or a pharmaceutically acceptable salt, hydrate or prodrug thereof to a human subject in need thereof, wherein R₁, R₂, R₃, R₃ and Rₓ are as defined herein.

The present invention also provides a method of treating or preventing dentatorubral-pallidoluysian atrophy (Haw River Syndrome), the method comprising administering an effective amount of a heterocyclic compound having the general Formula (I):

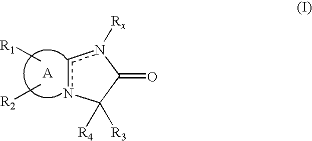

or a pharmaceutically acceptable salt, hydrate or prodrug thereof to a human subject in need thereof, wherein R₁, R₂, R₃, R₃ and Rₓ are as defined herein.

The present invention also provides a method of treating or preventing spinocerebellar ataxia, the method comprising administering an effective amount of a heterocyclic compound having the general Formula (I):

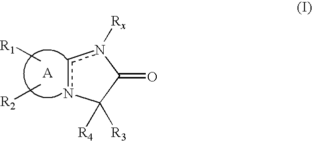

or a pharmaceutically acceptable salt, hydrate or prodrug thereof to a human subject in need thereof, wherein R₁, R₂, R₃, R₃ and Rₓ are as defined herein.

The present invention also provides a method of treating or preventing macular degeneration, the method comprising administering an effective amount of a heterocyclic compound having the general Formula (I):

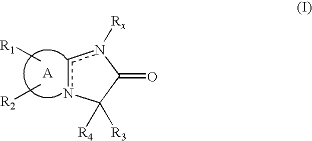

or a pharmaceutically acceptable salt, hydrate or prodrug thereof to a human subject in need thereof, wherein R₁, R₂, R₃, R₃ and Rₓ are as defined herein.

The present invention also provides a method of treating or preventing Parkinson's disease, the method comprising administering an effective amount of a heterocyclic compound having the general Formula (I):

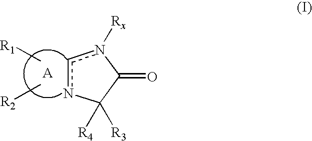

or a pharmaceutically acceptable salt, hydrate or prodrug thereof to a human subject in need thereof, wherein R₁, R₂, R₃, R₃ and Rₓ are as defined herein.

The present invention also provides a method of treating or preventing polyglutamine diseases, the method comprising administering to a subject in need thereof an effective amount of a heterocyclic compound having the general Formula (I):

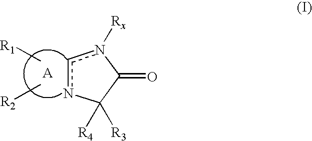

or a pharmaceutically acceptable salt, hydrate or prodrug thereof to a human subject in need thereof, wherein R₁, R₂, R₃, R₃ and Rₓ are as defined herein.

The present invention also provides a method of treating or preventing Huntington's disease, the method comprising administering to a subject in need thereof an effective amount of a heterocyclic compound having the general Formula (I):

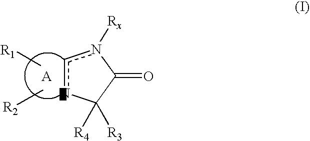

or a pharmaceutically acceptable salt, hydrate or prodrug thereof to a human subject in need thereof, wherein R₁, R₂, R₃, R₃ and Rₓ are as defined herein.

The present invention also provides a method of treating or preventing systemic amyloidosis, the method comprising administering to a subject in need thereof an effective amount of a heterocyclic compound having the general Formula (I):

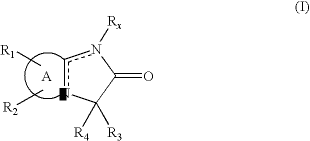

or a pharmaceutically acceptable salt, hydrate or prodrug thereof to a human subject in need thereof, wherein R₁, R₂, R₃, R₃ and Rₓ are as defined herein.

### BRIEF DESCRIPTION OF THE DRAWINGS/FIGURES

FIGS. 1A and 1B are bar graphs that depict the effect of the compound ST101 on Aβ production by Neuro2a (N2a) cells. FIG. 1A is a bar graph that depicts the Aβ concentration in the cell culture medium as a function of ST101 concentration compared to control after 24 hours of treatment. FIG. 1B is a bar graph that depicts the ratio of Aβ 1-42 to Aβ 1-40 as a function of ST101 concentration compared to control.

FIGS. 2A, 2B and 2C are graphs that depict the effect of ST101 in 3xTg-AD mice in the Morris water maze. FIG. 2A is a graph depicting latency (in seconds) during training over a period of seven days, compared to control mice. FIGS. 2B and 2C are graphs that depict latency (in seconds) and number of crosses over the platform location at 24 and 72 hours after training in ST101-treated animals and control mice.

FIGS. 3A and 3B are bar graphs that depict the effect of ST101 on Aβ in brain tissue from 3xTg-AD mice. FIG. 3A depicts the amounts of soluble Aβ₁₋₄₀ and Aβ₁₋₄₂ in brain tissue in mice treated with ST101, relative to control mice. FIG. 3B a bar graph that depicts the amounts of insoluble Aβ₁₋₄₀ and Aβ₁₋₄₂ (formic acid extraction) in mice treated with ST101, relative to control mice.

FIG. 4 is a Western blot that depicts APP carboxy-terminal fragments detected by antibody CT20 in the brains of ST101-treated (S) 3xTg-AD mice, relative to untreated (C) 3xTg-AD mice.

FIG. 5 is a Western blot that depicts APP and degradation fragments detected by antibody CT20 in the brains of ST101-treated (S) 3xTg-AD mice, relative to untreated (C) 3xTg-AD mice. * indicates full length APP species, ** indicates major degradation products, and “Actin” stands for anti-beta-actin antibody as a protein loading control.

FIG. 6 is a drawing that depicts a proposed amyloid processing pathway leading to a novel amyloid precursor protein carboxy-terminal fragment.

FIGS. 7A and 7B are bar graphs that depict the effect of ST101 on Aβ in brain tissue from 3xTg-AD mice. FIG. 7A depicts the amounts of soluble Aβ₁₋₄₀ and Aβ₁₋₄₂ in brain tissue in mice treated with ST101, relative to control mice. FIG. 7B is a bar graph that depicts the amounts of insoluble Aβ₁₋₄₀ and Aβ₁₋₄₂ (formic acid extraction) in mice treated with ST101, relative to control mice. * denotes a statistically significant difference from control animals.

FIGS. 8A and 8B are bar graphs that depict the effect of ST101 on Aβ in brain tissue from 3xTg-AD mice. FIG. 8A depicts the amounts of soluble Aβ₁₋₄₀ and Aβ₁₋₄₂ in brain tissue in mice treated with ST101, relative to control mice. FIG. 8B a bar depicts the amounts of insoluble Aβ₁₋₄₀ and Aβ₁₋₄₂ (formic acid extraction) in mice treated with ST101, relative to control mice.

FIG. 9 is a bar graph that depicts the effect of ST 101 on Aβ in brain tissue from cynomolgus monkeys. FIG. 9 depicts the amount of levels of Aβ₁₋₄₀ in monkeys treated with ST 101, relative to control monkeys.

FIGS. 10A-10D are Western blots that depict APP carboxy-terminal fragments detected in the brains of ST101-treated (T in FIG. 10A, S in FIGS. 10B-10C) 3xTg-AD mice, relative to untreated (C) 3xTg-AD mice. In FIGS. 10A and 10B, the CT20 antibody was used. FIG. 10B is from a separate experiment that used the same brain extract used in the experiment for FIG. 10A. In FIGS. 10C and 10D, an APP C-terminal antibody (Eptitomics #: 1565-1) was used. FIG. 10D is a lighter exposure of the Western blot in FIG. 10C.

FIG. 11A is a series of Western blots depicting levels of proADAM10, ADAM10, proBACE, BACE, Presenilinl and APP-CFTs in brain extracts from ST101 treated 3×TG-AD mice (S) versus control mice (C). FIG. 11B depicts quantification of the Western blot bands from FIG. 11A by densitometry.

FIG. 12 is a series of Western blots depicting levels of full-length tau, tau accumulates, tau degradation products and phosphorylated tau levels in brain extracts from ST101 treated 3×TG-AD mice (S) versus control mice (C). Beta actin levels (Ac) were used as a loading control.

FIG. 13 is a Western blot that shows the effect of ST101 on sAPP-beta in brain tissue from 3xTgAD mice.

FIG. 14 is a Western blot that shows the effect of ST101 on TACE in brain tissue from 3xTgAD mice.

FIG. 15 is a Western blot that shows the effect of ST101 on ubiquitylated protein levels in brain tissue from ST101-treated 3xTgAD mice (S) versus control mice (C).

FIG. 16A is Western Blot that shows the effect of ST101 on α-tubulin of higher molecular weight that is presumably ubiquitylated tubulin in brain tissue from 3xTgAD mice. FIG. 16B is a Western Blot that shows the effect of ST101 on levels of acetylated α-tubulin in brain tissue from ST101-treated 3xTgAD mice (S) versus control mice (C).

FIG. 17 is a Western Blot that shows the effect of ST101 on levels of huntingtin protein phosphorylated at Serine 13 in brain tissue from ST101-treated 3xTgAD mice (S) versus control mice (C).

FIG. 18 is a series of Western blots depicting levels of LC3, Cathepsin D, Beclin 1, and LAMP2A in brain extracts from ST101 treated 3×TG-AD mice (S) versus control mice (C).

FIGS. 19A-19C are a series of Western blots that depict APP carboxy-terminal fragments detected by APP antibody 1565-1 (Epitomics) in the brains of ST101-treated (S) cynomolgus monkeys, relative to untreated monkeys (C). FIG. 19B is bar graphs that depict the effect of ST101 on amounts of soluble Aβ₁₋₄₀ and Aβ₁₋₄₂ in brain tissue in male monkeys treated with ST101, relative to control monkeys. FIG. 19C is a Western blot that depicts BACE, ADAM10, GAPDH (loading control) and APP carboxy-terminal fragments (detected by APP antibody CT20) in the brains of ST101-treated monkeys, relative to untreated monkeys.

FIGS. 20A and 20B are Western blots that depict acetylated alpha-tubulin, beta-tubulin and their degradation fragment detected in the brains of ST101-treated (S) cynomolgus monkeys, relative to untreated monkeys (C).

FIG. 21A is a Western blot that depicts the effect of ST101 on ubiquitylated protein levels in brain tissue of ST101-treated (S) wildtype C57/B6 mice, relative to untreated mice (C). FIG. 21B is a bar graph that shows the quantification by densitometry of the ubiquitin signal in FIG. 21A.

FIG. 22 is a Western blot that depicts proteasome alpha subunits, proteasome beta subunits 21 and 51, and actin (loading control) detected in the brains of ST101-treated (S) cynomolgus monkeys, relative to untreated monkeys (C).

FIG. 23 is a Western blot that depicts APP carboxy-terminal fragments including the AICD fragment detected by APP antibody CT20 (Calbiochem) in the brains of ST101-treated (S) 3xTg-AD mice, relative to untreated (C) 3xTg-AD mice. * indicates the 17 kD fragment.

### DETAILED DESCRIPTION OF THE INVENTION

Unless defined otherwise, all technical and scientific terms used herein have the same meaning as is commonly understood by one of ordinary skill in the art to which this invention belongs.

The present invention provides a method of inducing cleavage of APP to produce an approximately 17 kDa carboxy-terminal fragment of APP in a subject, the method comprising administering a heterocyclic compound having the general Formula (I):

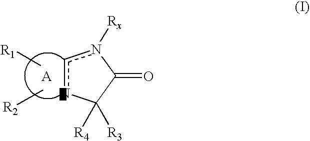

or a pharmaceutically acceptable salt, hydrate or prodrug thereof to a subject in need thereof, wherein the approximately 17 kDa fragment includes the carboxyterminal amino acid sequence of APP and amyloid-beta amino acid sequence.

In another embodiment, administering a heterocyclic compound having the general Formula (I) results in a decrease in the production of one or more of Aβ₁₋₄₂, Aβ₁₋₄₀, the C99 fragment of APP, and/or the C83 fragment of APP.

In another embodiment, administering a heterocyclic compound having the general Formula (I) results in a decrease in Aβ.

In another embodiment, the subject has Alzheimer's Disease-related pathology mediated cognitive decline in Down syndrome. In another embodiment, the Alzheimer's Disease-related pathology mediated cognitive decline in Down syndrome is treated. In another embodiment, the Alzheimer's Disease-related pathology mediated cognitive decline in Down syndrome is prevented.

In another embodiment, the subject to whom a heterocyclic compound having the general Formula (I) is administered has AD. In another embodiment, the subject has been diagnosed with AD. In another embodiment, the subject has mild cognitive impairment. In another embodiment, the subject has been diagnosed with mild cognitive impairment.

In another embodiment, the AD is treated. In another embodiment, the mild cognitive impairment is treated. As used herein, “treatment” means any manner in which the symptoms of a condition, disorder or disease are ameliorated or otherwise beneficially altered. In another embodiment, the subject has been diagnosed with AD.

In one embodiment, the AD is prevented. In another embodiment, the mild cognitive impairment is prevented. “Preventing” AD or cognitive impairment, as used herein, refers to preventing the occurrence of one or more symptoms of AD in a subject.

As used herein, amelioration of the symptoms of a particular disorder by administration of a particular pharmaceutical composition refers to any lessening, whether permanent or temporary, lasting or transient, that can be attributed to or associated with administration of the composition.

In another embodiment, the subject is screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is screened to determine whether the subject is predisposed to develop AD. The screening can be performed by examining the subject. Alternatively, the screening can be performed by assaying one or more biological markers of predisposition to AD.

The present invention also provides an isolated approximately 17 kDa APP fragment that includes the carboxy-terminal amino acid sequence of APP and amyloid-beta amino acid sequence.

The present invention also provides a composition comprising the approximately 17 kDA fragment of the invention. In another embodiment, the composition also comprises cell culture lysate and/or medium.

The present invention also provides a container comprising the approximately 17 kDA fragment of the invention. In another embodiment, the container is a microtube. In another embodiment, the container is a test tube. In another embodiment, the container is pipette or a micropipette. In another embodiment, the container is a microarray apparatus. In another embodiment, the container is a microtiter plate. In another embodiment, the container is a component of a screening assay apparatus.

The present invention also provides a method for screening for a compound that cleaves APP to generate an approximately 17 kDa fragment of APP, the method comprising: (a) exposing cells that produce APP or fragments thereof to a test compound, and (b) detecting the amount of the approximately 17 kDa fragment, wherein the approximately 17 kDa fragment includes the carboxy-terminal amino acid sequence of APP and amyloid-beta amino acid sequence, and wherein an increase in the amount of the approximately 17 kDa fragment of cells exposed to the compound, relative to the amount of the approximately 17 kDa fragment in cells that are not exposed to the compound, indicates that the compound induces cleavage of APP to generate the approximately 17 kDa fragment.

Alternatively, one can detect the presence of the free amino-terminus of the approximately 17 kDa fragment, or one can detect the free carboxy-terminus of APP generated by the cleavage that created the 17 kDa fragment.

The present invention also provides a method for screening for a compound that cleaves APP to generate an approximately 17 kDa fragment of APP, the method comprising: (a) exposing cells that produce APP or fragments thereof to a test compound, and (b) detecting the approximately 17 kDa fragment, wherein the approximately 17 kDa fragment includes the carboxy-terminal amino acid sequence of APP and amyloid-beta amino acid sequence, and wherein the presence of the approximately 17 kDa fragment of cells exposed to the compound, relative to the absence of the approximately 17 kDa fragment in cells that are not exposed to the compound, indicates that the compound induces cleavage of APP to generate the approximately 17 kDa fragment.

In one embodiment, the method further comprises (c) determining whether the amount of one or more of Aβ₁₋₄₂, Aβ₁₋₄₀, the C99 fragment of APP, or the C83 fragment of APP in cells exposed to the compound is decreased, relative to the amount of Aβ₁₋₄₂, Aβ₁₋₄₀, the C99 fragment of APP, or the C83 fragment of APP in cells that are not exposed to the compound.

In another embodiment, the screening method of the present invention is carried out in vitro. In this embodiment, the amount of the approximately 17 kDa fragment in the cell culture can be measured, for cells that are exposed to the compound and for control cells that are not exposed to the compound. An increase in the amount of the approximately 17 kDa fragment in the cell culture of cells exposed to the compound, relative to the amount of the approximately 17 kDa fragment in the cell culture of cells that are not exposed to the compound, indicates that the compound cleaves APP to generate the approximately 17 kDa fragment.

The approximately 17 kDa APP fragment, Aβ₁₋₄₂, Aβ₁₋₄₀, the C99 fragment of APP, or the C83 fragment of APP can also be detected, for example, using gel electrophoresis. The 17 kDa APP fragment, Aβ₁₋₄₂, Aβ₁₋₄₀, the C99 fragment of APP, or the C83 fragment of APP can also be detected using a sandwich ELISA assay employing a first monoclonal antibody directed against the N-terminus of the 17 kDa fragment and a second monoclonal antibody directed against another region of the 17 kDa fragment, for example, the carboxy-terminus of the 17 kDa fragment.

The approximately 17 kDa APP fragment, Aβ₁₋₄₂, Aβ₁₋₄₀, the C99 fragment of APP, or the C83 fragment of APP can also be detected, for example, using mass spectrometry, with or without prior immunoprecipitation by an antibody.

In another embodiment, the approximately 17 kDa fragment is isolated. The term “isolated” as used herein means separated from the brain of a subject. In another embodiment, the approximately 17 kDa fragment is present in an electrophoretic gel. In another embodiment, the approximately 17 kDa fragment is present in cell culture lysate or medium.

The “approximately 17 kDa fragment” of APP is the fragment of APP that contains the C-terminal sequence of APP and the amyloid-beta sequence of APP. The approximately 17 kDa fragment is not the C99 fragment of APP or the C83 fragment of APP.

The present invention also provides a method of inducing cleavage of APP to produce an approximately 17 kDa carboxy-terminal fragment of APP in a subject, the method comprising administering a compound that is not a compound having the general Formula (I):

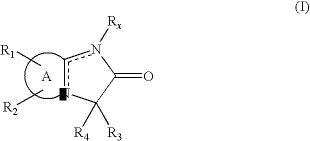

or a pharmaceutically acceptable salt, hydrate or prodrug thereof.

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan).

In another embodiment, administering a compound that is not a compound having the general Formula (I) results in a decrease in the production of one or more of Aβ₁₋₄₂, Aβ₁₋₄₀, the C99 fragment of APP, and/or the C83 fragment of APP.

In another embodiment, the subject to whom a heterocyclic compound having the general Formula (I) is administered has AD. In another embodiment, the subject has been diagnosed with AD. In another embodiment, the subject has mild cognitive impairment. In another embodiment, the subject has been diagnosed with mild cognitive impairment.

In another embodiment, the AD is treated. In another embodiment, the mild cognitive impairment is treated. As used herein, “treatment” means any manner in which the symptoms of a condition, disorder or disease are ameliorated or otherwise beneficially altered. In another embodiment, the subject has been diagnosed with AD.

In one embodiment, the AD is prevented. In another embodiment, the mild cognitive impairment is prevented. “Preventing” AD or cognitive impairment, as used herein, refers to preventing the occurrence of one or more symptoms of AD in a subject.

As used herein, amelioration of the symptoms of a particular disorder by administration of a particular pharmaceutical composition refers to any lessening, whether permanent or temporary, lasting or transient, that can be attributed to or associated with administration of the composition.

In another embodiment, the subject is screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is screened to determine whether the subject is predisposed to develop AD. The screening can be performed by examining the subject. Alternatively, the screening can be performed by assaying one or more biological markers of predisposition to AD.

The present invention also provides a method of decreasing the level of pro-ADAM10 and/or BACE protein in a subject, the method comprising administering a heterocyclic compound having the general Formula (I):

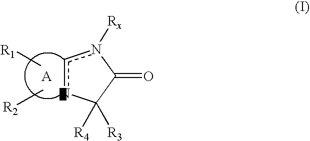

or a pharmaceutically acceptable salt, hydrate or prodrug thereof to a subject in need thereof, wherein R₁, R₂, R₃, R₃ and Rₓ are as defined herein.

In one embodiment, the level of pro-ADAM10 is decreased. In another embodiment, the level of BACE is decreased. In another embodiment, the level of pro-ADAM10 and the level of BACE are decreased.

Levels of pro-ADAM10 and BACE can be assayed, for example, in a Western blot using antibodies that are specific for pro-ADAM10 and BACE, respectively.

In one embodiment, pro-ADAM10 and/or BACE protein level is reduced in the brain of the subject.

In another embodiment, the subject to whom a heterocyclic compound having the general Formula (I) is administered has AD. In another embodiment, the subject has been diagnosed with AD. In another embodiment, the subject has mild cognitive impairment. In another embodiment, the subject has been diagnosed with mild cognitive impairment.

In another embodiment, the AD is treated. In another embodiment, the mild cognitive impairment is treated. In another embodiment, the subject has been diagnosed with AD.

In one embodiment, the AD is prevented. In another embodiment, the mild cognitive impairment is prevented.

In another embodiment, the subject is screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is screened to determine whether the subject is predisposed to develop AD. The screening can be performed by examining the subject. Alternatively, the screening can be performed by assaying one or more biological markers of predisposition to AD. In another embodiment, the subject has inclusion body myositis. In another embodiment, the inclusion body myositis is treated. In another embodiment, the inclusion body myositis is prevented.

In another embodiment, the subject has Alzheimer's Disease-related pathology mediated cognitive decline in Down syndrome. In another embodiment, the Alzheimer's Disease-related pathology mediated cognitive decline in Down syndrome is treated. In another embodiment, the Alzheimer's Disease-related pathology mediated cognitive decline in Down syndrome is prevented.

In another embodiment, administering the heterocyclic compound results in a decrease in the mRNA transcription of pro-ADAM 10 and/or BACE.

In another embodiment, administering the heterocyclic compound results in a decrease in the protein translation of pro-ADAM10 and/or BACE.

In another embodiment, administering the heterocyclic compound results in a post-translational modification of pro-ADAM10 and/or BACE.

In another embodiment, administering the heterocyclic compound results in increased degradation of pro-ADAM10 and/or BACE.

The present invention also provides a method for screening for a compound that decreases the level of pro-ADAM10 and/or BACE, the method comprising: (a) exposing cells or tissue that express pro-ADAM10 and/or BACE to a test compound, and (b) detecting the amount of pro-ADAM10 and/or BACE in the cells or tissue, wherein an decrease in the amount pro-ADAM10 and/or BACE protein in cells or tissue exposed to the compound, relative to pro-ADAM10 and/or BACE protein in cells or tissue that are not exposed to the compound, indicates that the compound decreased the amount of pro-ADAM10 and/or BACE protein.

The present invention also provides a method of decreasing the level of pro-ADAM10 and/or BACE protein in a subject, the method comprising administering a heterocyclic compound that is not a compound having the general Formula (I):

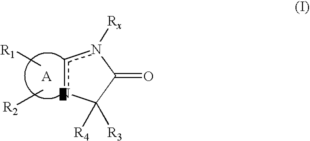

or a pharmaceutically acceptable salt, hydrate or prodrug thereof to a subject in need thereof, wherein R₁, R₂, R₃, R₃ and Rₓ are as defined herein.

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan).

In one embodiment, the level of pro-ADAM10 is decreased. In another embodiment, the level of BACE is decreased. In another embodiment, the level of pro-ADAM10 and the level of BACE are decreased.

In one embodiment, pro-ADAM10 and/or BACE protein level is reduced in the brain of the subject.

In another embodiment, the subject to whom a heterocyclic compound having the general Formula (I) is administered has AD. In another embodiment, the subject has been diagnosed with AD. In another embodiment, the subject has mild cognitive impairment. In another embodiment, the subject has been diagnosed with mild cognitive impairment.

In another embodiment, the AD is treated. In another embodiment, the mild cognitive impairment is treated. In another embodiment, the subject has been diagnosed with AD.

In one embodiment, the AD is prevented. In another embodiment, the mild cognitive impairment is prevented.

In another embodiment, the subject is screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is screened to determine whether the subject is predisposed to develop AD. The screening can be performed by examining the subject. Alternatively, the screening can be performed by assaying one or more biological markers of predisposition to AD.

In another embodiment, administering the heterocyclic compound results in a decrease in the mRNA transcription of pro-ADAM 10 and/or BACE.

In another embodiment, administering the heterocyclic compound results in a decrease in the protein translation of pro-ADAM10 and/or BACE.

In another embodiment, administering the heterocyclic compound results in a post-translational modification of pro-ADAM10 and/or BACE.

In another embodiment, administering the heterocyclic compound results in increased degradation of pro-ADAM10 and/or BACE.

In another embodiment, the subject to whom a heterocyclic compound having the general Formula (I) is administered has an inflammatory condition. In another embodiment, the subject has been diagnosed with an inflammatory condition. In another embodiment, the inflammatory condition is treated. In another embodiment, the inflammatory condition is prevented.

In another embodiment, the subject to whom a heterocyclic compound having the general Formula (I) is administered has cancer. In another embodiment, the subject has been diagnosed with cancer. In another embodiment, the cancer is treated. In another embodiment, the cancer is prevented.

In another embodiment, the subject to whom a heterocyclic compound having the general Formula (I) is administered has cystic fibrosis. In another embodiment, the subject has been diagnosed with cystic fibrosis. In another embodiment, the cystic fibrosis is treated. In another embodiment, the cystic fibrosis is prevented.

In another embodiment, the subject to whom a heterocyclic compound having the general Formula (I) is administered has an allergic condition. In another embodiment, the subject has been diagnosed with an allergic condition. In another embodiment, the allergic condition is treated. In another embodiment, the allergic condition is prevented.

The present invention also provides an isolated approximately 32 kDa phosphorylated tau protein fragment.

The present invention also provides a composition comprising an isolated approximately 32 kDa phosphorylated tau protein fragment. In another embodiment, the composition also comprises cell culture lysate and/or medium.

The present invention also provides a container comprising an isolated phosphorylated tau protein fragment. In another embodiment, the container is a microtube. In another embodiment, the container is a test tube. In another embodiment, the container is pipette or a micropipette. In another embodiment, the container is a microarray apparatus. In another embodiment, the container is a microtiter plate. In another embodiment, the container is a component of a screening assay apparatus.

The present invention also provides a method of decreasing tau protein accumulation in a subject, the method comprising administering a heterocyclic compound having the general Formula (I):

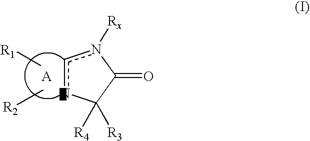

or a pharmaceutically acceptable salt, hydrate or prodrug thereof to a subject in need thereof, wherein R₁, R₂, R₃, R₃ and Rₓ are as defined herein.

Tau level can be assayed, for example, in a Western blot using an antibody that is specific for tau, or a specific ELISA.

In one embodiment, the subject to whom a heterocyclic compound having the general Formula (I) is administered has AD. In another embodiment, the subject has been diagnosed with AD. In another embodiment, the subject has mild cognitive impairment. In another embodiment, the subject has been diagnosed with mild cognitive impairment.

In another embodiment, the AD is treated. In another embodiment, the mild cognitive impairment is treated. In another embodiment, the subject has been diagnosed with AD.

In one embodiment, the AD is prevented. In another embodiment, the mild cognitive impairment is prevented.

In another embodiment, the subject is screened to determine whether the subject has AD. The screening can be performed by examining the subject. Alternatively, the screening can be performed by assaying one or more biological markers of AD.

In another embodiment, the subject has been diagnosed as predisposed to AD.

In another embodiment, the subject is screened to determine whether the subject is predisposed to develop AD. The screening can be performed by examining the subject. Alternatively, the screening can be performed by assaying one or more biological markers of predisposition to AD.

In another embodiment, frontal temporal dementia is treated. In another embodiment, frontal temporal dementia is prevented.

The present invention also provides a method for screening for a compound that decreases tau protein accumulation, the method comprising: (a) exposing cells or tissue that accumulate tau protein to a test compound, and (b) detecting the amount of tau protein accumulated in said cells or tissue, wherein a decrease in the amount of tau protein accumulation in cells or tissue exposed to the compound, relative to tau protein accumulation by cells or tissue that are not exposed to the compound, indicates that the compound decreased the amount of tau protein accumulation.

The present invention also provides a method of decreasing tau protein accumulation in a subject, the method comprising administering a heterocyclic compound that is not a compound having the general Formula (I):

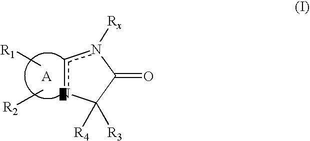

or a pharmaceutically acceptable salt, hydrate or prodrug thereof to a subject in need thereof, wherein R₁, R₂, R₃, R₃ and Rₓ are as defined herein, and wherein the compound is not a compound disclosed in International Application No. PCT/US2006/026331, which published as WO 2007/008586.

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan).

In one embodiment, the subject to whom a heterocyclic compound having the general Formula (I) is administered has AD. In another embodiment, the subject has been diagnosed with AD. In another embodiment, the subject has mild cognitive impairment. In another embodiment, the subject has been diagnosed with mild cognitive impairment.

In another embodiment, the AD is treated. In another embodiment, the mild cognitive impairment is treated. In another embodiment, the subject has been diagnosed with AD.

In one embodiment, the AD is prevented. In another embodiment, the mild cognitive impairment is prevented.

In another embodiment, the subject is screened to determine whether the subject has AD. The screening can be performed by examining the subject. Alternatively, the screening can be performed by assaying one or more biological markers of AD.

In another embodiment, the subject has been diagnosed as predisposed to AD. In another embodiment, the subject is screened to determine whether the subject is predisposed to develop AD. The screening can be performed by examining the subject. Alternatively, the screening can be performed by assaying one or more biological markers of predisposition to AD.

The present invention also provides a method of treating or preventing inflammation in a subject, the method comprising administering a heterocyclic compound having the general Formula (I):

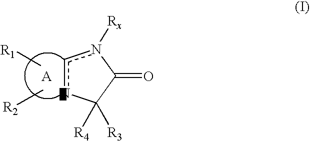

or a pharmaceutically acceptable salt, hydrate or prodrug thereof to a subject in need thereof, wherein R₁, R₂, R₃, R₃ and Rₓ are as defined herein.

In one embodiment, the subject has an inflammatory condition.

In another embodiment, the subject has been diagnosed with an inflammatory condition.

In another embodiment, the inflammatory condition is treated.

In another embodiment, the inflammatory condition is prevented.

In another embodiment, the inflammatory condition is selected from the group consisting of psoriasis, Crohn's disease, rheumatoid arthritis, asthma, an autoimmune disease, chronic inflammation, chronic prostatitis, glomerulonephritis, hypersensitivities, inflammatory bowel disease, pelvic inflammatory disease, reperfusion injury, rheumatoid arthritis, transplant rejection, inclusion body myositis, and vasculitis. Other inflammatory conditions not listed herein can be treated or prevented by the method of the present invention.

The present invention also provides a method of treating or preventing cystic fibrosis in a subject, the method comprising administering a heterocyclic compound having the general Formula (I):

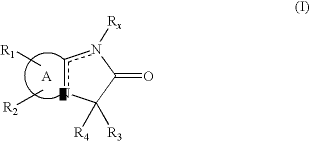

or a pharmaceutically acceptable salt, hydrate or prodrug thereof to a subject in need thereof, wherein R₁, R₂, R₃, R₃ and Rₓ are as defined herein.

In one embodiment, the subject has cystic fibrosis.

In another embodiment, the subject has been diagnosed with cystic fibrosis.

In another embodiment, the cystic fibrosis is treated.

In another embodiment, the cystic fibrosis is prevented.

In another embodiment, the heterocyclic compound is administered by inhalation.

The present invention also provides a method of treating or preventing a hyperproliferative disease in a subject, the method comprising administering a heterocyclic compound having the general Formula (I):

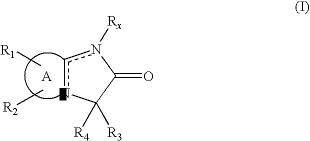

or a pharmaceutically acceptable salt, hydrate or prodrug thereof to a subject in need thereof, wherein R₁, R₂, R₃, R₃ and Rₓ are as defined herein.

In one embodiment, the hyperproliferative disease is cancer. In another embodiment, the cancer is treated.

In another embodiment, the subject has been diagnosed with cancer.

In another embodiment, the inflammatory condition is treated.

In another embodiment, the subject has been diagnosed as predisposed to cancer.

In another embodiment, then subject has been screened to determine whether the subject is predisposed to cancer.

In another embodiment, the cancer is selected from the group of breast cancer, lymphoma, skin cancer, pancreatic cancer, colon cancer, rectal cancer, pancreatic cancer, kidney cancer, skin cancer, leukemia, thyroid cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. Other cancers listed herein can be treated or prevented by the method of the present invention.

The present invention also provides a method of treating or preventing allergy in a subject, the method comprising administering a heterocyclic compound having the general Formula (I):

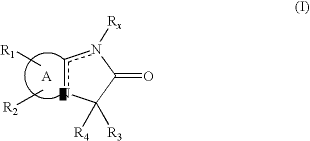

or a pharmaceutically acceptable salt, hydrate or prodrug thereof to a subject in need thereof, wherein R₁, R₂, R₃, R₃ and Rₓ are as defined herein.

In one embodiment, the subject has one or more allergies.

In another embodiment, the subject has been diagnosed with one or more allergies.

In another embodiment, the one or more allergies is treated.

In another embodiment, the one or more allergies is prevented.

In another embodiment, the heterocyclic compound is administered by inhalation.

In another embodiment, the allergic condition is selected from the group consisting of allergic asthma, perennial allergic rhinitis, seasonal allergic rhinitis, atopic dermatitis, contact hypersensitivity, contact dermatitis, conjunctivitis, allergic conjunctivitis, eosinophilic bronchitis, food allergies, eosinophilic gastroenteritis, inflammatory bowel disease, ulcerative colitis, Crohn's disease, mastocytosis, hyper IgE syndrome, systemic lupus erythematus, psoriasis, acne, multiple sclerosis, allograft rejection, reperfusion injury, chronic obstructive pulmonary disease, rheumatoid arthritis, psoriatic arthritis and osteoarthritis, an animal allergy, a venom allergy, a plant allergy an anaphylactic reaction, and a hypersensitivity reaction. In one embodiment, the allergic condition is a local allergic condition. In another embodiment, the allergic condition is a systemic allergic condition. Other allergic conditions not listed herein can be treated or prevented by the method of the present invention.

The present invention also provides a method of reducing levels of ubiquitylated proteins in a human subject, the method comprising administering a heterocyclic compound having the general Formula (I):

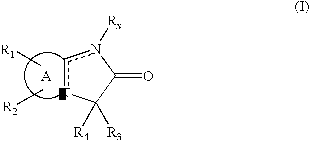

or a pharmaceutically acceptable salt, hydrate or prodrug thereof to a subject in need thereof, wherein R₁, R₂, R₃, R₃ and Rₓ are as defined herein.

In one embodiment, the ubiquitylated protein is reduced in at least one tissue or organ of a subject, e.g. in the brain, muscle tissue or mucosal tissue of the subject.

In one embodiment, the subject to whom a heterocyclic compound having the general Formula (I) is administered has Alzheimer's Disease. In another embodiment, the subject has been diagnosed with Alzheimer's Disease. In another embodiment, the Alzheimer's disease is treated. In another embodiment, the Alzheimer's Disease is prevented. In another embodiment, the subject has been diagnosed as predisposed to Alzheimer's Disease. In another embodiment, the subject has been screened to determine whether the subject is predisposed to Alzheimer's Disease.

In one embodiment, the subject to whom a heterocyclic compound having the general Formula (I) is administered has mild cognitive impairment. In another embodiment, the subject has been diagnosed with mild cognitive impairment. In another embodiment, the mild cognitive impairment is treated. In another embodiment, the mild cognitive impairment is prevented.

In one embodiment, the subject to whom a heterocyclic compound having the general Formula (I) is administered has Parkinson's disease. In another embodiment, the subject has been diagnosed with Parkinson's disease. In another embodiment, the Parkinson's disease is treated. In another embodiment, the Parkinson's disease is prevented.

In another embodiment, the subject to whom a heterocyclic compound having the general Formula (I) is administered has Huntington's disease. In another embodiment, the subject has been diagnosed with Huntington's disease. In another embodiment, the Huntington's disease is treated. In another embodiment, the Huntington's disease is prevented.

The present invention also provides a method of reducing levels of ubiquitylated proteins in a human subject, the method comprising administering a compound that is not a heterocyclic compound having the general Formula (I):

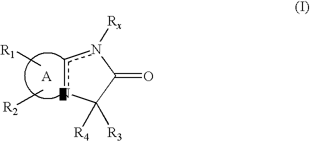

or a pharmaceutically acceptable salt, hydrate or prodrug thereof to a subject in need thereof, wherein R₁, R₂, R₃, R₃ and Rₓ are as defined herein, wherein the compound is not a ubiquitin hydrolase. In one embodiment, the heterocyclic compound is not spiro(imidazo[1,2-a]pyridin-2(3H)-one-3,2′-indan).

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan).

In one embodiment, the ubiquitylated protein is reduced in the brain of the subject.

In one embodiment, the subject to whom a heterocyclic compound having the general Formula (I) is administered has Alzheimer's Disease. In another embodiment, the subject has been diagnosed with Alzheimer's Disease. In another embodiment, the Alzheimer's disease is treated. In another embodiment, the Alzheimer's Disease is prevented. In another embodiment, the subject has been diagnosed as predisposed to Alzheimer's Disease. In another embodiment, the subject has been screened to determine whether the subject is predisposed to Alzheimer's Disease.

In one embodiment, the subject to whom a heterocyclic compound having the general Formula (I) is administered has mild cognitive impairment. In another embodiment, the subject has been diagnosed with mild cognitive impairment. In another embodiment, the mild cognitive impairment is treated. In another embodiment, the mild cognitive impairment is prevented.

In one embodiment, the subject to whom a heterocyclic compound having the general Formula (I) is administered has Parkinson's disease. In another embodiment, the subject has been diagnosed with Parkinson's disease. In another embodiment, the Parkinson's disease is treated. In another embodiment, the Parkinson's disease is prevented.

The present invention also provides a method of enhancing the removal and degradation of harmful proteins in a human subject, the method comprising administering a heterocyclic compound having the general Formula (I):

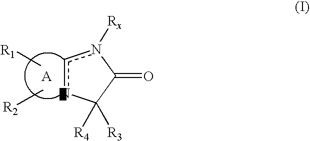

or a pharmaceutically acceptable salt, hydrate or prodrug thereof to a human subject in need thereof, wherein R₁, R₂, R₃, R₃ and Rₓ are as defined herein.

The present invention also provides a method of enhancing the removal and degradation of harmful proteins in a human subject, the method comprising administering a compound that is not a heterocyclic compound having the general Formula (I):

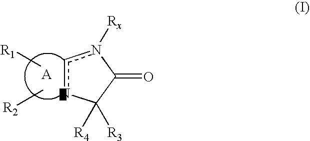

or a pharmaceutically acceptable salt, hydrate or prodrug thereof to a human subject in need thereof, wherein R₁, R₂, R₃, R₃ and Rₓ are as defined herein. In one embodiment, the heterocyclic compound is not spiro(imidazo[1,2-a]pyridin-2(3H)-one-3,2′-indan).

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan).

The present invention also provides a method of enhancing proteasome breakdown of accumulated and/or harmful proteins in a human subject, the method comprising administering a heterocyclic compound having the general Formula (I):

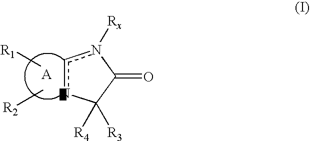

or a pharmaceutically acceptable salt, hydrate or prodrug thereof to a human subject in need thereof, wherein R₁, R₂, R₃, R₃ and Rₓ are as defined herein.

The present invention also provides a method of enhancing proteasome breakdown of accumulated and/or harmful proteins in a human subject, the method comprising administering a compound that is not a heterocyclic compound having the general Formula (I):

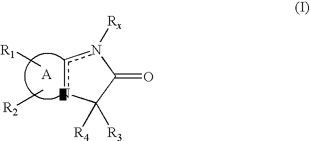

or a pharmaceutically acceptable salt, hydrate or prodrug thereof to a human subject in need thereof, wherein R₁, R₂, R₃, R₃ and Rₓ are as defined herein, and wherein the compound is not a ubiquitin hydrolase.

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan)

The present invention also provides a method of enhancing the activity of the ubiquitin-proteasome system pathway in a human subject, the method comprising administering a heterocyclic compound having the general Formula (I):

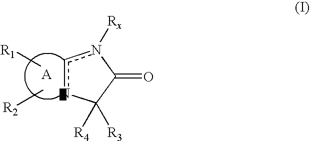

or a pharmaceutically acceptable salt, hydrate or prodrug thereof to a human subject in need thereof, wherein R₁, R₂, R₃, R₃ and Rₓ are as defined herein.

The present invention also provides a method of enhancing the activity of the ubiquitin-proteasome system pathway in a human subject, the method comprising administering a compound that is not a heterocyclic compound having the general Formula (I):

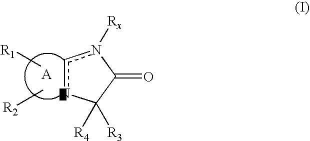

or a pharmaceutically acceptable salt, hydrate or prodrug thereof to a human subject in need thereof, wherein R₁, R₂, R₃, R₃ and Rₓ are as defined herein. In one embodiment, the heterocyclic compound is not spiro(imidazo[1,2-a]pyridin-2(3H)-one-3,2′-indan), and wherein the compound is not a ubiquitin hydrolase.

In one embodiment, the compound is not a compound disclosed in any of U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety. In another embodiment, the compound is not spiro(imidazo[1,2-a]pyridin-2(3H)-one-3,2′-indan)

The present invention also provides a method of treating or preventing a prion disease, the method comprising administering a heterocyclic compound having the general Formula (I):

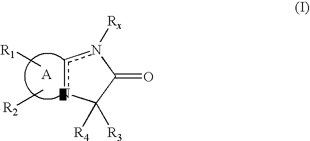

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has prion disease wherein a PrP variant is misfolded. In another embodiment, the subject has been diagnosed with prion disease. In another embodiment, the prion disease is treated. In another embodiment, the prion disease is prevented.

The present invention also provides a method of treating or preventing cataracts of the eye, the method comprising administering a heterocyclic compound having the general Formula (I):

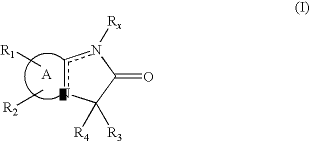

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has cataracts of the eye. In another embodiment, the subject has been diagnosed with cataracts of the eye. In another embodiment, the cataracts of the eye are treated. In another embodiment, the cataracts of the eye are prevented.

The present invention also provides a method of treating or preventing type 2 diabetes, the method comprising administering a heterocyclic compound having the general Formula (I):

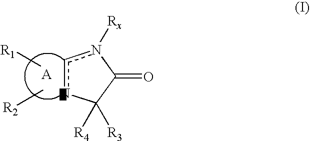

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has type 2 diabetes. In another embodiment, the subject has been diagnosed with type 2 diabetes. In another embodiment, the type 2 diabetes is treated. In another embodiment, the type 2 diabetes is prevented.

The present invention also provides a method of treating or preventing Paget's disease, the method comprising administering a heterocyclic compound having the general Formula (I):

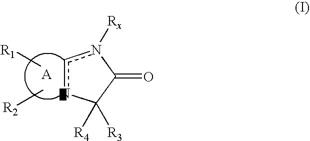

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has Paget's disease. In another embodiment, the subject has been diagnosed with Paget's disease. In another embodiment, the Paget's disease is treated. In another embodiment, the Paget's disease is prevented.

The present invention also provides a method of treating or preventing

Amyotrophic Lateral Sclerosis, the method comprising administering a heterocyclic compound having the general Formula (I):

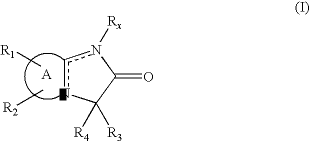

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has Amyotrophic Lateral Sclerosis. In another embodiment, the subject has been diagnosed with Amyotrophic Lateral Sclerosis. In another embodiment, the Amyotrophic Lateral Sclerosis treated. In another embodiment, the Amyotrophic Lateral Sclerosis is prevented.

The present invention also provides a method of treating or preventing frontotemporolobar dementia, the method comprising administering a heterocyclic compound having the general Formula (I):

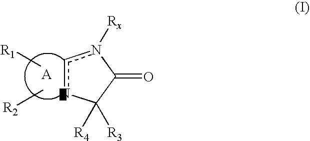

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has frontotemporolobar dementia. In another embodiment, the subject has been diagnosed with frontotemporolobar dementia. In another embodiment, the frontotemporolobar dementia is treated. In another embodiment, the frontotemporolobar dementia is prevented.

The present invention also provides a method of treating or preventing Lewy body disease, the method comprising administering a heterocyclic compound having the general Formula (I):

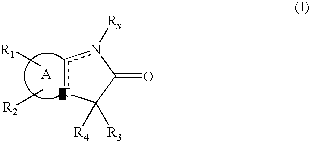

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has Lewy body disease. In another embodiment, the subject has been diagnosed with Lewy body disease. In another embodiment, the Lewy body disease treated. In another embodiment, the Lewy body disease is prevented.

The present invention also provides a method of treating or preventing sporadic inclusion body myositis, the method comprising administering a heterocyclic compound having the general Formula (I):

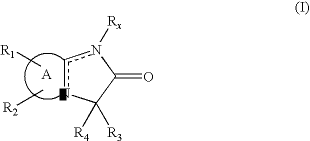

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has sporadic inclusion body myositis. In another embodiment, the subject has been diagnosed with sporadic inclusion body myositis. In another embodiment, the sporadic inclusion body myositis treated. In another embodiment, the sporadic inclusion body myositis is prevented.

The present invention also provides a method of treating traumatic brain injury, the method comprising administering a heterocyclic compound having the general Formula (I):

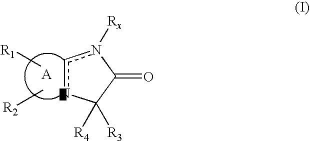

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has suffered a traumatic brain injury.

The present invention also provides a method of treating or preventing cardiac dysfunction, the method comprising administering a heterocyclic compound having the general Formula (I):

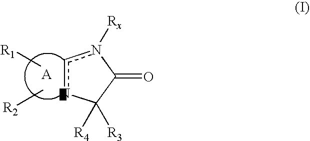

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has cardiac dysfunction. In another embodiment, the subject has been diagnosed with cardiac dysfunction. In another embodiment, the cardiac dysfunction is treated. In another embodiment, the cardiac dysfunction is prevented. In one embodiment, the cardiac dysfunction is heart failure, e.g., congestive heart failure. In another embodiment, the cardiac dysfunction is cardiac hypertrophy.

The present invention also provides a method of treating or preventing spinobulbar muscular atrophy (Kennedy Disease), the method comprising administering a heterocyclic compound having the general Formula (I):

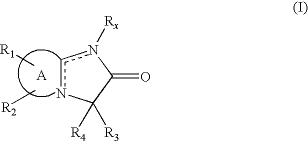

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has spinobulbar muscular atrophy. In another embodiment, the subject has been diagnosed with spinobulbar muscular atrophy. In another embodiment, the spinobulbar muscular atrophy is treated. In another embodiment, the spinobulbar muscular atrophy is prevented.

The present invention also provides a method of treating or preventing dentatorubral-pallidoluysian atrophy (Haw River Syndrome), the method comprising administering a heterocyclic compound having the general Formula (I):

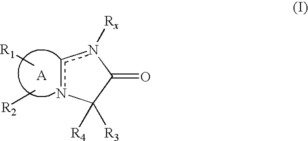

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has dentatorubral-pallidoluysian atrophy. In another embodiment, the subject has been diagnosed with dentatorubral-pallidoluysian atrophy. In another embodiment, the dentatorubral-pallidoluysian atrophy is treated. In another embodiment, the dentatorubral-pallidoluysian atrophy is prevented.

The present invention also provides a method of treating or preventing spinocerebellar ataxia, the method comprising administering a heterocyclic compound having the general Formula (I):

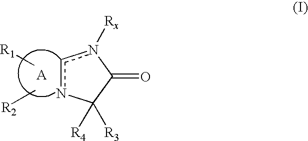

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has spinocerebellar ataxia. In another embodiment, the subject has been diagnosed with spinocerebellar ataxia. In another embodiment, the spinocerebellar ataxia is treated. In another embodiment, the spinocerebellar ataxia is prevented. In one embodiment, the spinocerebellar ataxia is spinocerebellar ataxia type 1. In another embodiment, the spinocerebellar ataxia is spinocerebellar ataxia type 2. In another embodiment, the spinocerebellar ataxia is spinocerebellar ataxia type 3. In another embodiment, the spinocerebellar ataxia is spinocerebellar ataxia type 6. In another embodiment, the spinocerebellar ataxia is spinocerebellar ataxia type 7. In another embodiment, the spinocerebellar ataxia is spinocerebellar ataxia type 17.

The present invention also provides a method of treating or preventing macular degeneration, the method comprising administering a heterocyclic compound having the general Formula (I):

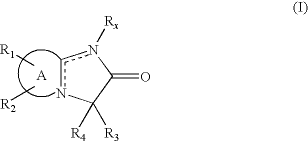

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has macular degeneration. In another embodiment, the subject has been diagnosed with macular degeneration. In another embodiment, the macular degeneration is treated.

The present invention also provides a method of treating or preventing Parkinson's disease, the method comprising administering a heterocyclic compound having the general Formula (I):

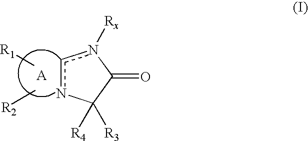

or a pharmaceutically acceptable salt, hydrate or prodrug thereof to a human subject in need thereof, wherein R₁, R₂, R₃, R₃ and Rₓ are as defined herein. In one embodiment, the subject to whom a heterocyclic compound having the general Formula (I) is administered has Parkinson's disease. In another embodiment, the subject has been diagnosed with Parkinson's disease. In another embodiment, the Parkinson's disease is treated.

The present invention also provides a method of treating or preventing polyglutamine diseases, the method comprising administering to a subject in need there of an effective amount of a heterocyclic compound having the general Formula (I):

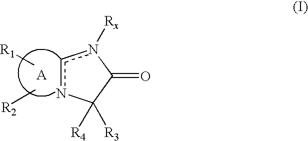

or a pharmaceutically acceptable salt, hydrate or prodrug thereof to a human subject in need thereof, wherein R₁, R₂, R₃, R₃ and Rₓ are as defined herein.

The present invention also provides a method of treating or preventing systemic amyloidosis, the method comprising administering to a subject in need there of an effective amount of a heterocyclic compound having the general Formula (I):

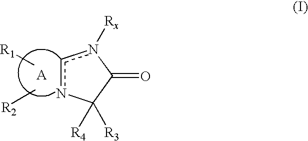

or a pharmaceutically acceptable salt, hydrate or prodrug thereof to a human subject in need thereof, wherein R₁, R₂, R₃, R₃ and Rₓ are as defined herein.

In one embodiment of any of the methods herein, the subject is a human subject.

In one embodiment of any of the methods herein, ubiquitylated protein is mono-ubiquitylated. In another embodiment of any of the methods herein, ubiquitylated protein is poly-ubiquitylated.

The term “harmful protein,” as used herein, refers to a protein that is damaged, misfolded, toxic, or unwanted.

The present invention also provides a method for screening for a compound that decreases the level of ubiquitylated protein, the method comprising: (a) exposing cells or tissue that express ubiquitylated protein to a test compound, and (b) detecting the amount of ubiquitylated protein in said cells or tissue, wherein an decrease in the amount ubiquitylated protein in cells or tissue exposed to the compound, relative to ubiquitylated protein in cells or tissue that are not exposed to the compound, indicates that the compound decreased the amount of ubiquitylated protein.

In one embodiment of any of the screening methods herein, the screening method is carried out in vivo. In another embodiment, the screening method is carried out in vitro.

In another embodiment of any of the screening methods herein, the screening method is carried out in a high-throughput manner. In another embodiment, the screening method is automated. In another embodiment, the screening method invention is computer-controlled.

In another embodiment of any of the screening methods herein, the screening method is carried out in the brain of an animal. In another embodiment, the cells are in the brain of an animal.

In another embodiment of any of the screening methods herein, the screening method is carried out in cells in cell culture or tissue culture. In another embodiment, the cells are selected from the group consisting of SHSY5Y, HEK, PC12, CHO, fibroblast, 3T3, IMR-32, BV-2, T98G, NT2N, Neuro2A cells, primary neuronal cells, and primary microglial cells, and organotypic slice cultures from wild-type or transgenic mice. In another embodiment, the cells are Neuro2A cells.

In another embodiment of any of the screening methods herein, the screening method is carried out in a high-throughput manner. In another embodiment, the screening method is computer-controlled.

In another embodiment of any of the screening methods herein, the compound screened is a small molecule. In another embodiment, the compound screened is a nucleic acid. In another embodiment, the compound screened is an antisense-RNA molecule, an RNAi molecule, an interfering RNA molecule, a small interfering RNA molecule, or an siRNA molecule. In another embodiment, the compound screened is not one or more of an antisense-RNA molecule, an RNAi molecule, an interfering RNA molecule, a small interfering RNA molecule, or an siRNA molecule.

In another embodiment of any of the screening methods herein, a plurality of cultured cells are exposed separately to a plurality of test compounds, e.g. in separate wells of a microtiter plate. In this embodiment, a large number of test compounds may be screened at the same time.

The test compounds may be presented to the cells or cell lines dissolved in a solvent. Examples of solvents include, DMSO, water and/or buffers. DMSO may be used in an amount below about 1%. Alternatively, DMSO may be used in an amount of about 0.1% or below. At this concentration, DMSO functions as a solubilizer for the test compounds and not as a permeabilization agent. The amount of solvent tolerated by the cells must be checked initially by measuring cell viability with the different amounts of solvent alone to ensure that the amount of solvent has no effect on the cellular properties being measured.

Suitable buffers include cellular growth media, for example Iscove's media (Invitrogen Corporation) with or without 10% fetal bovine serum. Other known cellular incubation buffers include phosphate, PIPES or HEPES buffers. One of ordinary skill in the art can identify other suitable buffers with no more than routine experimentation.

Cells that produce APP or fragments thereof include, but are not limited to SHSY5Y, HEK, PC12, CHO, fibroblast, 3T3, IMR-32, BV-2, T98G, NT2N, Neuro2A cells, primary neuronal cells, and primary microglial cells. In another embodiment, the cells are Neuro2A cells.

In another embodiment, the cells that produce APP or fragments thereof include cells into which nucleic acid encoding APP or mutated APP has been introduced, e.g., by transfection.

The heterocyclic compound of the present invention can be administered at an effective oral dosage of 0.0005 mg per kilogram of body weight or higher. In one embodiment, the compound is administered as part of a unit dosage form containing 5, 10, 15, 20, 25, 30, 35, 40, 45, 50, 55, 60, 65, 70, 75, 80, 85, 90, 95, or 100 mg.

Compositions for use in this invention include all compositions wherein the active ingredient is contained in an amount which is effective to achieve its intended purpose. While individual needs vary, determination of optimal ranges of effective amounts of each component is within the skill of the art. Typically, the active ingredient may be administered to mammals, e.g. humans, orally at a dose of 0.001 to 3 mg/kg, or an equivalent amount of the pharmaceutically acceptable salt thereof, per day of the body weight of the mammal being treated for AD. The active ingredient may be administered to mammals, e.g. humans, intravenously or intramuscularly at a dose of 0.001 to 3 mg/kg, or an equivalent amount of the pharmaceutically acceptable salt thereof, per day of the body weight of the mammal being treated for AD. Approximately 0.001 to approximately 3 mg/kg can be orally administered to treat or prevent such disorders. If another agent is also administered, it can be administered in an amount which is effective to achieve its intended purpose.

The unit oral dose may comprise from approximately 0.001 to approximately 200 mg, or approximately 0.5 to approximately 180 mg of the composition of the invention. The unit dose may be administered one or more times daily as one or more tablets, each containing from approximately 0.1 to approximately 90 mg, conveniently approximately 10 to 180 mg of the composition or its solvates. In one embodiment, the unit oral dose can be 10, 20, 30, 40, 50, 60, 70, 80, 90, 100, 110, 120, 130, 140, 150, 160, 170, or 180 mg.

In a topical formulation, the active ingredient may be present at a concentration of approximately 0.01 to 100 mg per gram of carrier.

In addition to administering the active ingredient as a raw chemical, the active ingredient may be administered as part of a pharmaceutical preparation containing suitable pharmaceutically acceptable carriers comprising excipients and auxiliaries, which facilitate processing of the active ingredient into preparations that can be used pharmaceutically. The preparations, particularly those preparations, which can be administered orally, such as tablets, dragees, and capsules, and also preparations, which can be administered rectally, such as suppositories, as well as suitable solutions for administration by injection or orally, can contain from approximately 0.01 to 99 percent, or from approximately 0.25 to 75 percent of active ingredient, together with the excipient.

The heterocyclic compound of Formula (I) can be in the form of hydrate or acid addition salts as a pharmaceutically acceptable salt. Possible acid addition salts include inorganic acid salts such as the hydrochloride, sulfate, hydrobromide, nitrate, and phosphate salts and organic acid salts such as acetate, oxalate, propionate, glycolate, lactate, pyruvate, malonate, succinate, maleate, fumarate, malate, tartrate, citrate, benzoate, cinnamate, methanesulfonate, benzenesulfonate, p-toluenesulfonate, and salicylate salts.

Acid addition salts are formed by mixing a solution of the particular compound of the present invention with a solution of a pharmaceutically acceptable non-toxic acid, such as hydrochloric acid, hydrobromic acid, fumaric acid, maleic acid, succinic acid, acetic acid, citric acid, lactic acid, tartaric acid, carbonic acid, phosphoric acid, sulfuric acid, oxalic acid, and the like. Basic salts are formed by mixing a solution of the particular compound of the present invention with a solution of a pharmaceutically acceptable non-toxic base, such as sodium hydroxide, potassium hydroxide, choline hydroxide, sodium carbonate, Tris, N-methyl-glucamine and the like.

The pharmaceutical compositions of the invention may be administered to any animal or “subject,” which may experience the beneficial effects of the active ingredient. Foremost among such subject animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal, inhalation, or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use can be obtained by combining the active ingredient with solid excipients, optionally grinding the resultant mixture and processing the mixture of granules, after adding suitable auxiliaries, if desired or necessary, to obtain tablets or dragee cores.

Suitable excipients are, in particular: fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, using, e.g. maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, e.g. silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active ingredient doses.

Other pharmaceutical preparations, which can be used orally, include push-fit capsules made of gelatin, as well as soft, sealed capsules made of gelatin and a plasticizer, such as glycerol or sorbitol. The push-fit capsules can contain the active ingredient in the form of granules, which may be mixed with fillers, such as lactose, binders such as starches, and/or lubricants such as talc or magnesium stearate and, optionally, stabilizers. In soft capsules, the active ingredient can be dissolved or suspended in suitable liquids, such as fatty oils, or liquid paraffin. In addition, stabilizers may be added.

Possible pharmaceutical preparations, which can be used rectally include, e.g. suppositories, which consist of a combination of one or more of the active ingredient with a suppository base. Suitable suppository bases are, e.g. natural or synthetic triglycerides, or paraffin hydrocarbons. In addition, it is also possible to use gelatin rectal capsules, which consist of a combination of the active ingredient with a base. Possible base materials include, e.g. liquid triglycerides, polyethylene glycols, or paraffin hydrocarbons.

Suitable formulations for parenteral administration include aqueous solutions of the active ingredient in water-soluble form, e.g. water-soluble salts and alkaline solutions. In addition, suspensions of the active ingredient as appropriate oily injection suspensions may be administered. Suitable lipophilic solvents or vehicles include fatty oils, e.g. sesame oil; or synthetic fatty acid esters, e.g. ethyl oleate or triglycerides or polyethylene glycol-400. Aqueous injection suspensions may contain substances, which increase the viscosity of the suspension include, e.g. sodium carboxymethyl cellulose, sorbitol, and/or dextran. Optionally, the suspension may also contain stabilizers.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady, Medicinal Chemistry: A Biochemical Approach, Oxford University Press, New York, pages 388 392 (1985)).

Also included within the scope of the present invention are dosage forms of the active ingredient, in which the oral pharmaceutical preparations comprise an enteric coating. The term “enteric coating” is used herein to refer to any coating over an oral pharmaceutical dosage form that inhibits dissolution of the active ingredient in acidic media, but dissolves rapidly in neutral to alkaline media and has good stability to long-term storage. Alternatively, the dosage form having an enteric coating may also comprise a water soluble separating layer between the enteric coating and the core.

The core of the enterically coated dosage form comprises an active ingredient. Optionally, the core also comprises pharmaceutical additives and/or excipients. The separating layer may be a water soluble inert active ingredient or polymer for film coating applications. The separating layer is applied over the core by any conventional coating technique known to one of ordinary skill in the art. Examples of separating layers include, but are not limited to sugars, polyethylene glycol, polyvinylpyrrolidone, polyvinyl alcohol, hydroxypropyl cellulose, polyvinyl acetal diethylaminoacetate and hydroxypropyl methylcellulose. The enteric coating is applied over the separating layer by any conventional coating technique. Examples of enteric coatings include, but are not limited to cellulose acetate phthalate, hydroxypropyl methylcellulose phthalate, polyvinyl acetate phthalate, carboxymethylethylcellulose, copolymers of methacrylic acid and methacrylic acid methyl esters, such as Eudragit®L 12,5 or Eudragit®L 100 (Rohm Pharma), water based dispersions such as Aquateric® (FMC Corporation), Eudragit®L 100-55 (Rohm Pharma) and Coating CE 5142 (BASF), and those containing water soluble plasticizers such as Citroflex® (Pfizer). The final dosage form is an enteric coated tablet, capsule or pellet.

Examples of prodrugs of the compounds of the invention include the simple esters of carboxylic acid containing compounds (e.g. those obtained by condensation with a C1-4 alcohol according to methods known in the art); esters of hydroxy containing compounds (e.g. those obtained by condensation with a C₁₋₄ carboxylic acid, C₃₋₆ dioic acid or anhydride thereof (e.g. succinic and fumaric anhydrides according to methods known in the art); imines of amino containing compounds (e.g. those obtained by condensation with a C₁₋₄ aldehyde or ketone according to methods known in the art); and acetals and ketals of alcohol containing compounds (e.g. those obtained by condensation with chloromethyl methyl ether or chloromethyl ethyl ether according to methods known in the art).

Symptoms of AD include confusion, disturbances in short-term memory, problems with attention, problems with spatial orientation, personality changes, language difficulties and mood swings. It is understood that the list of symptoms of AD may be expanded upon in the future as medical science continues to evolve. Thus, the term “symptoms of AD” is not to be limited to the list of symptoms provided herein.

As used herein an effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the disease. Typically, repeated administration is required to achieve the desired amelioration of symptoms.

In the general Formula (I), the structural unit having the general Formula (II) may be one or more structural units selected from multiple types of structural units having the general Formula (III).

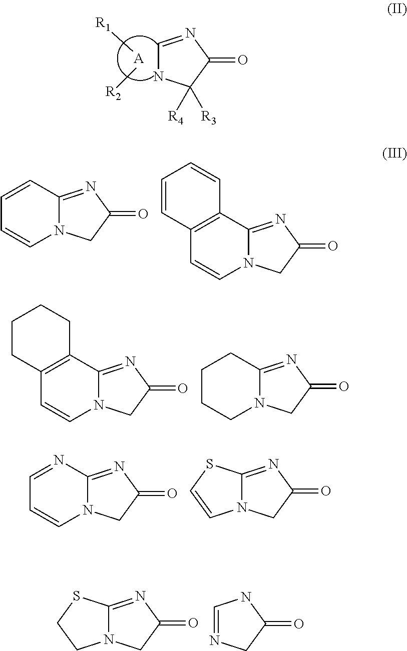

In the general Formula (I), Rₓ is methyl or nil. In the general Formula (I) and Formula (II), R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅ (wherein R₅ is phenyl (which may be substituted with C₁-C₆ alkyl, halogen atom or cyano) or thienyl) and —O—(CH₂)ₙ—R₆, wherein R₆ is a vinyl group, C₃-C₈ cycloalkyl group, or phenyl group, and n is 0 or 1.

In the general Formula (I) and Formula (II), R₃ and R₄ each are one or more functional groups independently selected from the group consisting of a hydrogen atom, C₁-C₆ alkyl group, C₂-C₆ alkenyl, C₃-C₈ cycloalkyl group, CH₂—R₅ (wherein R₅ is phenyl (which may be substituted with C₁-C₆ alkyl, halogen atom or cyano); naphtyl or thienyl) and —CH(R₈)—R₇. Alternatively, R₃ and R₄ together form a spiro ring having the general Formula (IV):

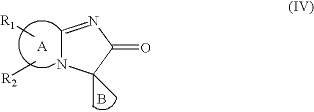

R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group. The above R₈ is a hydrogen atom or C₁-C₆ alkyl group.

Furthermore, in the general Formula (IV), the structural unit B may be one or more structural units selected from multiple types of structural units having the general Formula (V). The structural unit B binds at a position marked by * in the general Formula (V) to form a spiro ring.

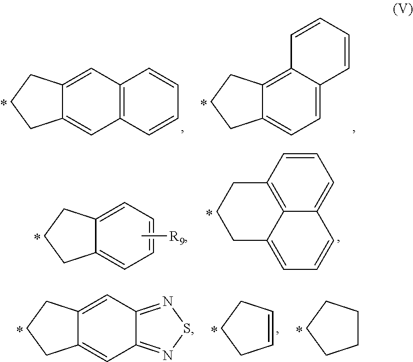

R₉ is one or more functional groups selected from the group consisting of a hydrogen atom, halogen atom, hydroxy group, C₁-C₆ alkoxy group, cyano group, and trifluoromethyl group.

When the heterocyclic compound having the general Formula (I) has asymmetric carbon atoms in the structure, its isomer from asymmetric carbon atoms and their mixture (racemic modification) is present. In such cases, all of them are included in the heterocyclic compound used in the embodiments described later.

The term “C₁-C₆” refers to 1 to 6 carbon atoms unless otherwise defined. The term “C₃-C₈” refers to 3 to 8 carbon atoms unless otherwise defined. The term “C₁-C₆ alkyl” includes linear or branched alkyl groups such as methyl, ethyl, n-propyl, isopropyl, n-butyl, tert-butyl, sec-butyl, n-pentyl, and n-hexyl. The term “C₁-C₆ alkoxy” includes linear or branched alkoxy groups such as methoxy, ethoxy, n-propoxy, isopropoxy, n-butoxy, tert-butoxy, sec-butoxy, n-pentyloxy, and n-hexyloxy. The term “C₃-C₈ cycloalkyl” includes cyclopropyl, cyclobutyl, cyclopentyl, cyclohexyl, cycloheptyl, and cyclooctyl. The term “halogen atom” includes fluorine, chlorine, bromine, and iodine.

In another embodiment, the heterocyclic compound useful in the practice of the present invention selected from the group consisting of:
3,3-dimethylimidazo(1,2-a)pyridin-2(3H)-one,
3,3-dipropylimidazo(1,2-a)pyridin-2(3H)-one,
3,3-dibutylimidazo(1,2-a)pyridin-2(3H)-one,
3,3-diallylimidazo(1,2-a)pyridin-2(3H)-one,
3,3-diallyl-8-benzyloxyimidazo(1,2-a)pyridin-2(3H)-one,
3,3-di(2-propinyl)imidazo(1,2-a)pyridin-2(3H)-one,
3,3-dibenzylimidazo(1,2-a)pyridin-2(3H)-one,
3,3-dibenzyl-8-methylimidazo(1,2-a)pyridin-2(3H)-one,
3,3-dibenzyl-5,7-dimethylimidazo(1,2-a)pyridin-2(3H)-one,
3,3-dibenzyl-8-hydroxyimidazo(1,2-a)pyridin-2(3H)-one,
3,3-dibenzyl-8-methoxyimidazo(1,2-a)pyridin-2(3H)-one,
3,3-dibenzyl-8-ethoxyimidazo(1,2-a)pyridin-2(3H)-one,
8-allyloxy-3,3-dibenzylimidazo(1,2-a)pyridin-2(3H)-one,
3,3-dibenzyl-8-isopropoxyimidazo(1,2-a)pyridin-2(3H)-one,
3,3-dibenzyl-8-cyclopropylmethyloxyimidazo(1,2-a)pyridin-2(3H)-one,
3,3-dibenzyl-8-cycloheptyloxyimidazo(1,2-a)pyridin-2(3H)-one,
3,3-dibenzyl-6-chloroimidazo[1,2-a]pyridin-2(3H)-one,
3,3-dibenzyl-6,8-dichloroimidazo[1,2-a]pyridin-2(3H)-one,
3,3-dibenzyl-8-chloro-6-trifluoromethylimidazo[1,2-a]pyridin-2(3H)-one,
3,3-dibenzyl-8-benzyloxyimidazo[1,2-a]pyridin-2(3H)-one,
8-amino-3,3-dibenzylimidazo[1,2-a]pyridin-2(3H)-one,
8-acetylamino-3,3-dibenzylimidazo[1,2-a]pyridin-2(3H)-one,
3,3-dibenzyl-8-benzylaminoimidazo[1,2-a]pyridin-2(3H)-one,
3,3-bis(3-chlorobenzyl)imidazo[1,2-a]pyridin-2(3H)-one,
3,3-bis(3-fluorobenzyl)imidazo[1,2-a]pyridin-2(3H)-one,
3,3-bis(4-fluorobenzyl)imidazo[1,2-a]pyridin-2(3H)-one,
3,3-bis(2,4-dichlorobenzyl)imidazo[1,2-a]pyridin-2(3H)-one,
3,3-bis(4-dimethylaminobenzyl)imidazo[1,2-a]pyridin-2(3H)-one,
3,3-bis(4-methoxybenzyl)imidazo[1,2-a]pyridin-2(3H)-one,
3,3-bis(4-biphenylmethyl)imidazo[1,2-a]pyridin-2(3H)-one,
3,3-bis(4-cyanobenzyl)imidazo[1,2-a]pyridin-2(3H)-one,
3,3-bis(4-hydroxy-benzyl)imidazo[1,2-a]pyridin-2(3H)-one,
3,3-bis(3-phenyl-1-propyl)imidazo[1,2-a]pyridin-2(3H)-one,
3,3-bis(2,4-difluorobenzyl)imidazo[1,2-a]pyridin-2(3H)-one,
3,3-bis(4-nitrobenzyl)imidazo[1,2-a]pyridin-2(3H)-one,
3,3-bis(4-carboxybenzyl)imidazo[1,2-a]pyridin-2(3H)-one,
8-benzyloxy-3,3-bis(1-phenylethyl)imidazo[1,2-a]pyridin-2(3H)-one,
8-benzyloxy-3,3-bis(3-methylbenzyl)imidazo[1,2-a]pyridin-2(3H)-one,
8-benzyloxy-3,3-bis(4-methylbenzyl)imidazo[1,2-a]pyridin-2(3H)-one,
3-benzyl-3-(4-fluorobenzyl)imidazo[1,2-a]pyridin-2(3H)-one,
3-ethyl-3(4-fluorobenzyl)imidazo[1,2-a]pyridin-2(3H)-one,
8-methyl-3,3-bis(3-pyridylmethyl)imidazo[1,2-a]pyridin-2(3H)-one,
8-methyl-3,3-bis(4-pyridylmethyl)imidazo[1,2-a]pyridin-2(3H)-one,
3,3-bis(2-thienylmethyl)imidazo[1,2-a]pyridin-2(3H)-one,
3,3-bis(2-furylmethyl)imidazo[1,2-a]pyridin-2(3H)-one,
spiro(imidazo[1,2-a]pyridin-2(3H)-one-3,2′-indan),
spiro(imidazo[1,2-a]pyridin-2(3H)-one-3,2′-(2,3)dihydrophenarene),
spiro(imidazo[2,1-b]thiazol-6(5H)-one-5,2′-benzo(f)indan),
spiro(imidazo[1,2-b]thiazol-6(5H)-one-5,2′-indan),
spiro(2-methylimidazo[1,2-b]thiazol-6(5H)-one-5,2′-benzo(f)indan),
5,5-bis(4-fluorobenzyl)imidazo[2,1-b]thiazol-6(5H)-one,
5,5-dibenzylimidazo[2,1-b]thiazol-6(5H)-one,
5,5-bis(4-methylbenzyl)imidazo[2,1-b]thiazol-6(5H)-one,
5,5-bis(4-cyanobenzyl)imidazo[2,1-b]thiazol-6(5H)-one,
5,5-dibenzyl-2-methylimidazo[2,1-b]thiazol-6(5H)-one,
5,5-bis(4-fluorobenzyl)-2-methylimidazo[2,1-b]thiazol-6(5H)-one,
5,5-dicyclohexyl-2-methylimidazo[2,1-b]thiazol-6(5H)-one,
5,5-bis(4-cyanobenzyl)-2-methylimidazo[2,1-b]thiazol-6(5H)-one,
5,5-di(2-butenyl)imidazo[2,1-b]thiazol-6(5H)-one,
5,5-dibutylimidazo[2,1-b]thiazol-6(5H)-one,
5,5-dicyclohexylimidazo[2,1-b]thiazol-6(5H)-one,
5,5-bis(2-thienylmethyl)imidazo[2,1-b]thiazol-6(5H)-one,
spiro(2,3-dihydroimidazo[2,1-b]thiazol-6(5H)-one-5,2′-benzo(f)indan),
5,5-dibutyl-2,3-dihydroimidazo[2,1-b]thiazol-6(5H)-one,
5,5-di(2-butenyl)-2,3-dihydroimidazo[2,1-b]thiazol-6(5H)-one,
5,5-bis(4-methylbenzyl)-2,3-dihydroimidazo[2,1-b]thiazol-6(5H)-one,
5,5-bis(2-thienylmethyl)-2,3-dihydroimidazo[2,1-b]thiazol-6(5H)-one,
5,5-bis(4-fluorobenzyl)-2,3-dihydroimidazo[2,1-b]thiazol-6(5H)-one,
5,5-dibenzyl-2,3-dihydroimidazo[2,1-b]thiazol-6(5H)-one,
spiro(imidazo[1,2-a]pyridin-2(3H)-one-3,2′-benzo(f)indan),
2-hydroxy-3-(2-naphthylmethyl)-imidazo[1,2-a]pyridine,
3-benzylimidazo[1,2-a]pyridin-2(3H)-one,
spiro(5,6,7,8-tetrahydroimidazo[1,2-a]pyridin-2(3H)-one-3,2′-benzo(f)indan),
3,3-dicyclohexyl-5,6,7,8-tetrahydroimidazo[1,2-a]pyridin-2(3H)-one,
3,3-bis(2-thienylmethyl)-5,6,7,8-tetrahydroimidazo[1,2-a]pyridin-2(3H)-one,
3,3-dibutyl-5,6,7,8-tetrahydroimidazo[1,2-a]pyridin-2(3H)-one,
3,3-dipropyl-5,6,7,8-tetrahydroimidazo[1,2-a]pyridin-2(3H)-one,
spiro(imidazo[1,2-a]pyrimidin-2(3H)-one-3,2′-benzo(f)indan),
3,3-di(2-butenyl)imidazo[1,2-a]pyrimidin-2(3H)-one,
3,3-bis(2-thienylmethyl)imidazo[1,2-a]pyrimidin-2(3H)-one,
3,3-bis(4-fluorobenzyl)imidazo[1,2-a]pyrimidin-2(3H)-one,
3,3-dicyclohexylimidazo[1,2-a]pyrimidin-2(3H)-one,
3,3-bis(4-cyanobenzyl)imidazo[1,2-a]pyrimidin-2(3H)-one,
3,3-bis(4-methylbenzyl)imidazo[1,2-a]pyrimidin-2(3H)-one,
4,4-dibenzyl-1-methyl-5-oxo-4,5-dihydroimidazole,
spiro(imidazo[1,2-a]pyridin-2(3H)-one-3,2′-(4′-fluoroindan)),
spiro(imidazo[1,2-a]pyridin-2(3H)-one-3,2′-(5′-methoxyindan)),
spiro(imidazo[1,2-a]pyridin-2(3H)-one-3,2′-(5′-iodoindan)),
spiro(imidazo[1,2-a]pyridin-2(3H)-one-3,2′-(4′-cyanoindan)),
spiro(imidazo[2,1-a]isoquinolin-2(3H)-one-3,2′-indan),
spiro(imidazo[1,2-a]pyridin-2(3H)-one-3,2′-((1,2,5-thiadiazo)(4,5-c)indan)),
spiro(imidazo[2,1-a]isoquinolin-2(3H)-one-3,2′-((1,2,5-thiadiazo)(4,5-c)indan)),
spiro(imidazo[1,2-a]pyrimidin-2(3H)-one-3,4′-(1-cyclopentene)),
spiro(imidazo[1,2-a]pyrimidin-2(3H)-one-3,2′-indan),
spiro(imidazo[1,2-a]pyrimidin-2(3H)-one-3,2′-((1,2,5-thiadiazo)(4,5-c)indan)),
spiro(imidazo[1,2-a]pyridin-2(3H)-one-3,2′-(5′-trifluoromethylindan)),
spiro(imidazo[1,2-a]pyridin-2(3H)-one-3,2′-benzo(e)indan),
spiro(imidazo[2,1-a]isoquinolin-2(3H)-one-3,1′-(3′-cyclopentene)),
spiro(8-benzyloxyimidazo[1,2-a]pyridin-2(3H)-one-3,1′-(3′-cyclopentene)),
spiro(7,8,9,10-tetrahydroimidazo[2,1-a]isoquinolin-2(3H)-one-3,1′-cyclopentane),
spiro(imidazo[2,1-a]isoquinolin-2(3H)-one-3,1′-cyclopentane), and
spiro(5,6,7,8-tetrahydroimidazo[1,2-a]pyridin-2(3H)-one-3,2′-indan).

In another embodiment, the compound is spiro(imidazo[1,2-a]pyridin-2(3H)-one-3,2′-indan).

In another embodiment, the method of the present invention can be practiced using any of the compounds disclosed in U.S. application Ser. No. 11/872,408 (published as US 2008/0103157 A1); U.S. application Ser. No. 11/872,418 (published as US 2008/0103158 A1); U.S. Pat. No. 6,635,652; U.S. Pat. No. 7,141,579; and international Appl. No. PCT/JP2007/070962 (published as WO 2008/047951), each of which is incorporated by reference in its entirety.

The compound ST101, also know as ZSET1446, has shown pharmacological activity in rodent models of learning and memory relevant to AD after both acute (single-dose) and chronic administration. The chemical name for ST101 is spiro(imidazo[1,2-a]pyridin-2(3H)-one-3,2′-indan).

For example, ST101 significantly improves age-impaired memory and attenuates memory deficits induced by chemical amnesic agents such as methamphetamine, the glutamate receptor antagonist, MK-801 and the muscarinic antagonist, scopolamine. (Yamaguchi Y., et al., J. Pharmacol. Exp. Ther. 317:1079-87 (2006); Ito Y., et al., J. Pharmacol. Exp. Ther. 320: 819-27 (2007)).

Experiments have shown that ST101 potentiates nicotine-stimulated release of acetylcholine (ACh), increases extracellular ACh concentrations in the cerebral cortex, and increases extracellular concentrations of both ACh and dopamine in the hippocampus. The breadth of models across which ST101 exerts its effects suggests the potential for involvement at an upstream target in the signaling pathway(s) associated with these processes.

ST101 has also demonstrated effects in the Senescence Accelerated Mouse 8 (SAMP8), a mouse strain that develops age-related deficits in learning and memory along with accumulation of Aβ-like deposits in brain tissue. The SAMP8 mouse is discussed in Morley, J.E., Biogerontology 3: 57-60 (2002). ST101 decreased accumulation of Aβ-like deposits and also produced an improvement in learning and memory functions, suggesting the behavioral effect of ST101 may be linked to reduction of Aβ production and/or accumulation. See US 2008/103158 A1.

All patents, patent applications, and publications discussed herein are hereby incorporated by reference in their entireties.

### Example 1

### Effect of ST101 on β Amyloid In Vitro in Neuro2a Cultured Cells

Neuro2a is a murine neuroblastoma cell line that is known to produce amyloid peptides Aβ₁₋₄₀ and Aβ₁₋₄₂ in amounts measurable by ELISA assays. These forms of Aβ have been correlated with the pathology in AD brain and Aβ₁₋₄₂ in particular is postulated to have the ability to block a7 nicotinic receptors and to produce direct neurotoxic effects. Neuro2a cells were treated for 24 hours with ST101 added to the tissue culture medium. Tissue culture medium was collected and analyzed by ELISA for the presence of Aβ.

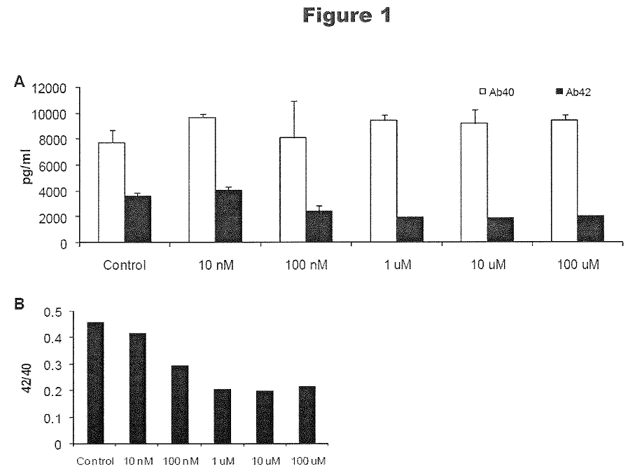

FIGS. 1A and 1B are bar graphs that depict the effect of the compound ST101 on Aβ production by Neuro2a cells. FIG. 1A is a bar graph that depicts the Aβ concentration in the cell culture medium as a function of ST101 concentration compared to control. FIG. 1B a bar graph that depicts the ratio of Aβ₁₋₄₂ to Aβ₁₋₄₀ as a function of ST101 concentration compared to control. As shown in FIGS. 1A and 1B, ST101 significantly reduced Aβ₁₋₄₂ without major effects on Aβ₁₋₄₀ (FIG. 1).

### Example 2

### Effect of ST101 in 3xTg-AD Mice in the Morrris Water Maze

Dr. Frank LaFerla's laboratory at the University of California, Irvine, has developed a transgenic mouse that contains 3 mutations relevant to Alzheimer's pathology (βAPPSwe, PS1M146V, and tauP301L) (Oddo et al., “Triple-transgenic model of AD with plaques and tangles: intracellular Aβ and synaptic dysfunction, Neuron 39(3):409-21 (2003)). These mutations shift APP cleavage from α- to β-secretase, increase production of Aβ₁₋₄₂ and drive the accumulation of tau into paired-helical filaments. The 3xTg-AD animals develop essential features of AD in an age-dependent fashion, with deficits in memory-related behavioral function, plaque and tangle pathology and synaptic dysfunction, including deficits in long-term potentiation, an activity believed critical to memory (Oddo et al., 2003). Furthermore, plaque formation precedes tangle formation and so mimics the development of the AD in humans. The 3xTg-AD mouse represents one of the closest animal models of AD developed to date.

### ST101 Administration and Test Methods

3xTg-AD mice of approximately one year of age were treated for 2 months with ST101. An average dose of 5 mg/kg/day was administered in drinking water (calculated dose, based on mean water consumption). Behavioral effects were tested by assessing performance on the Morris Water Maze. Biochemical effects were examined by measuring brain content of Aβ and APP by ELISA and Western Blot.

Behavioral Effects: Performance of 3xTg-AD Mice in the Morris Water Maze (MWM), adapted from Roozendaal et al., Proc. Natl. Acad. Sci. U.S.A. 100: 1328-1333 (2003).

The MWM tests both spatial memory (i.e. hippocampus dependent) and cued learning (i.e. non-hippocampal) in rodents. The maze is a circular tank filled with opaque water. Mice are placed in the water and must swim to find and escape onto a platform submerged 1.5 cm beneath the surface of the water. The time (in seconds) required to find the platform is recorded. Animals rely on visual cues in the room containing the tank in order to find the platform on successive challenges. Training was conducted daily for seven consecutive days.

Retention of training was assessed twice, 24 and 72 hours after the final training trial. Animals were subjected to a 60-second free swim in the tank with the platform removed. Parameters measured included (1) latency: time required to reach the former platform location and (2) crosses: the number of times the animal swam across the former platform location. Decreases in latency and increases in crosses are indicative of improved spatial memory and cued learning.

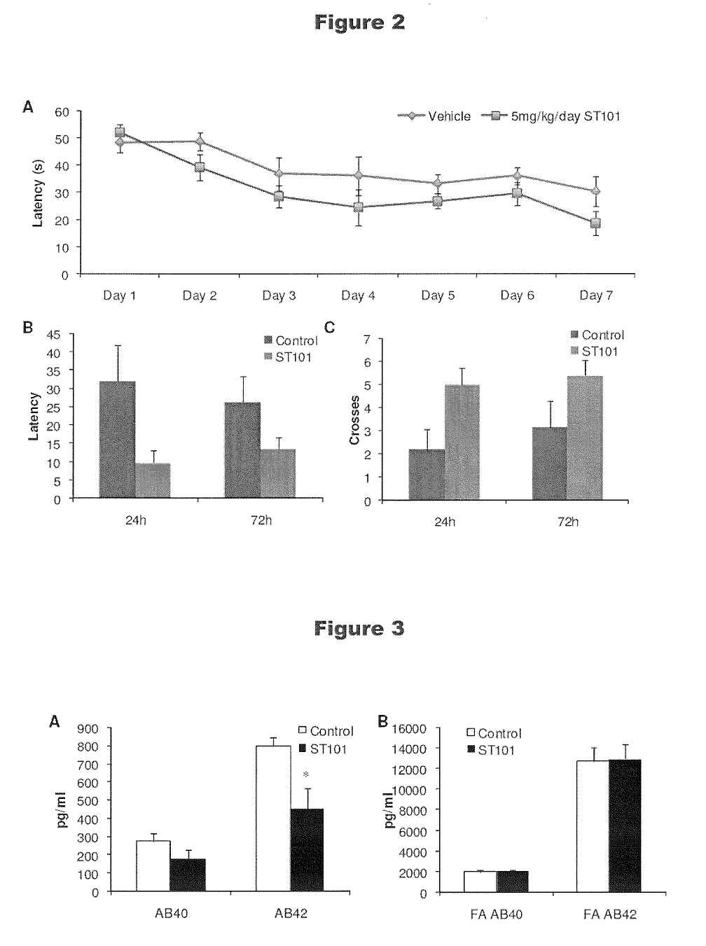

FIGS. 2A, 2B and 2C are graphs that depict the effect of ST101 in 3xTg-AD mice in the MWM. FIG. 2A is a graph depicting latency (in seconds) during training, compared to control mice. FIGS. 2B and 2C are bar graphs that depict latency (in seconds) at 24 and 72 hours after training in ST101-treated animals and control mice.

As shown in FIG. 2A, ST101 and Control animals had similar latency on the first day of training. However, ST 101-treated mice showed greater reductions in latency on successive days of the training compared with controls. FIGS. 2B and 2C also demonstrate both reductions in latency and increases in crosses during retention testing at both 24 and 72 hours. These data confirm that ST101 improves learning and memory performance in the 3xTg-AD mouse strain, which closely resembles human AD.

### Example 3

### Effect of ST101 on Aβ in Brain Tissue from 3xTg Mice-Ad

### Biochemical Effects: ST101 and Amyloid Processing Pathways

At the end of the 2-month treatment period, 3xTg Mice were sacrificed and brain tissue was processed. In the first set of analyses, soluble Aβ₁₋₄₀ and Aβ₁₋₄₂, as well as insoluble Aβ (after formic acid extraction), were quantified by ELISA. Soluble Aβ represents protein that has been processed from full length APP and released. Insoluble Aβ represents fibrillar accumulates that are ultimately deposited in amyloid plaques.

FIGS. 3A and 3B are bar graphs that depict the effect of ST101 on Aβ in brain tissue from 3xTg mice-AD. FIG. 3A depicts the amounts of soluble Aβ₁₋₄₀ and Aβ₁₋₄₂ in brain tissue in mice treated with ST101, relative to control mice. FIG. 3B a bar graph that depicts the amounts of insoluble Aβ₁₋₄₀ and Aβ₁₋₄₂ (formic acid extraction) in mice treated with ST101, relative to control mice. One animal in the ST101 treated group in panel A was excluded due to analytical artifact.

As shown in FIGS. 3A and 3B, ST101-treated mice had significantly decreased levels of soluble Aβ 1-42 and moderately decreased soluble Aβ₁₋₄₀. Insoluble Aβ was unaffected. These results suggest that ST101 may impact Aβ production or release.

### Example 4

### APP C-Terminal Fragments Detected by Antibody CT20

To attempt to determine at what part in the Aβ processing/release pathway ST101 may be active, a series of Western blot analyses of brain extracts from the same mice were conducted. These Westerns blots examined intact APP as well as products of its post-translational processing and subsequent degradation.

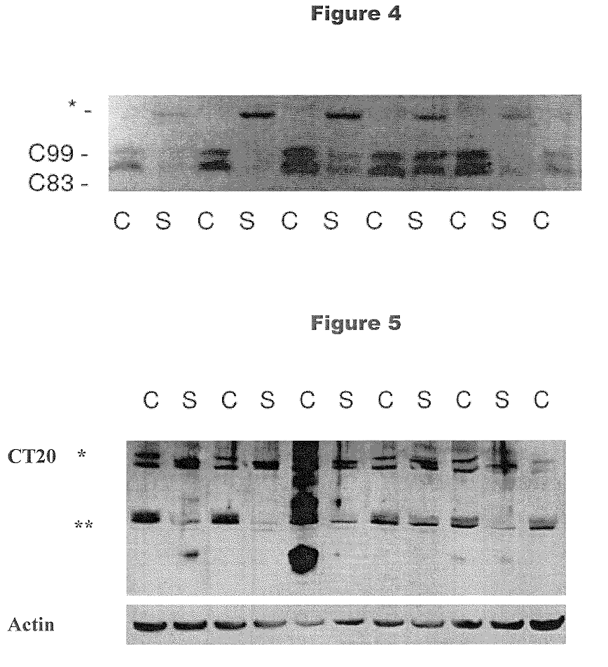

FIG. 4 is a Western blot that depicts APP C-terminal fragments detected by antibody CT20 in the brains of ST101-treated (S) 3xTg-AD mice, relative to untreated (C) 3xTg mice-AD.

As shown in FIG. 4, antibody CT20 (directed against the C-terminus of APP) revealed a substantial decrease in C99 and C83 C-terminal APP fragments. These fragments are byproducts of β-secretase and α-secretase cleavage, respectively. Also shown is the appearance of a novel, longer C-terminal fragment of about 17 kDa molecular weight (indicated by *).

### Example 5

### APP and Degradation Fragment Detected by Antibody CT20

FIG. 5 is a Western blot that depicts APP and degradation fragments detected by antibody CT20 in the brains of ST101-treated (S) 3xTg-AD mice, relative to untreated (C) 3xTg-AD mice. “CT20” stands for full length APP species, and “Actin” stands for anti-beta-actin antibody as a protein loading control.

The Western blot analysis detected full-length unprocessed APP in all extracts (FIG. 5,*). Subtle band shifts suggested additional ST101-induced modification of APP, e.g., slightly lowered molecular weight of some full-length species (possible change in glycosylation, phosphorylation or other post-translational modifications) and the disappearance or significant reduction of a major APP degradation intermediate (˜50 kDa) (FIG. 5, **).

### Example 6

### Alternative Amyloid Processing Pathway

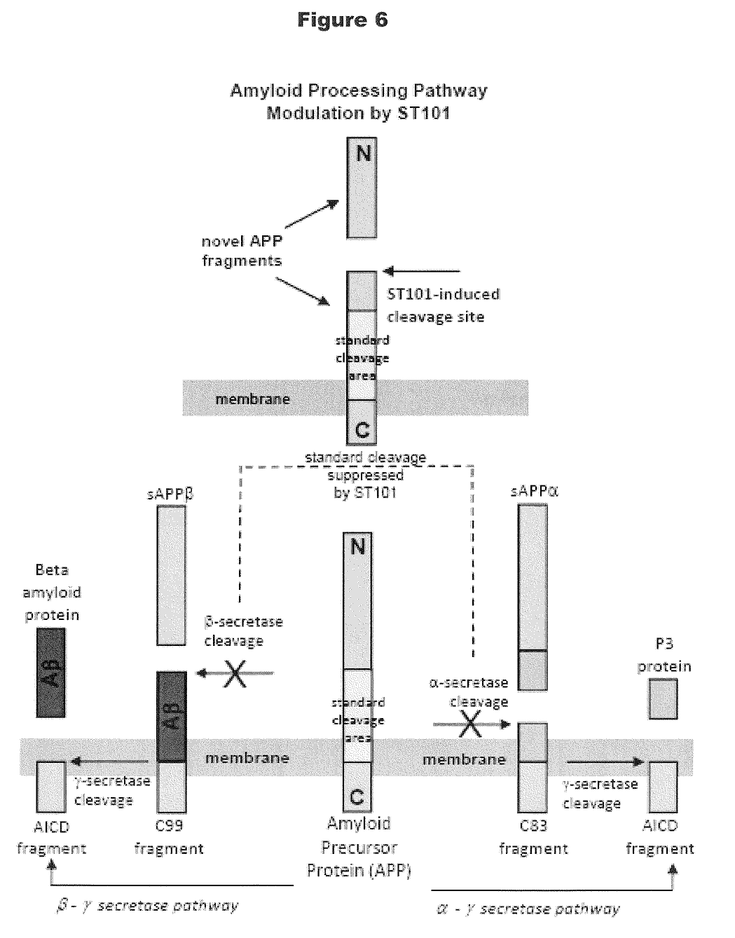

FIG. 6 is a drawing that depicts a proposed amyloid processing pathway leading to a novel APP C-terminal fragment. The proposed pathway explains the appearance of the novel approximately 17 kD fragment shown in the Western blot from FIG. 4. This fragment is generated by cleavage at an uncharacterized site about 60 amino acids N-terminal to the β-secretase cleavage site.

The new pathway appears to preempt both α- and β-secretase cleavage, as the usual products of these cleavage events are greatly reduced (C83 and C99 for α- and β-secretase, respectively), and therefore the cleavage sites that are the targets for these enzymes remain intact.

This alteration of APP metabolism induced by ST101 is accompanied by marked improvement in learning and memory tasks in an animal model arguably considered to be a close representation of clinical AD. When viewed in conjunction with earlier non-clinical data, it appears ST101 may operate at physiological processes upstream of those of both marketed agents and agents currently under investigation with known mechanisms of action and thus, represents a new avenue of treatment for AD.

### Example 7

### Effects of ST101 on Aβ Production In Vivo (3xTg-AD Mice)

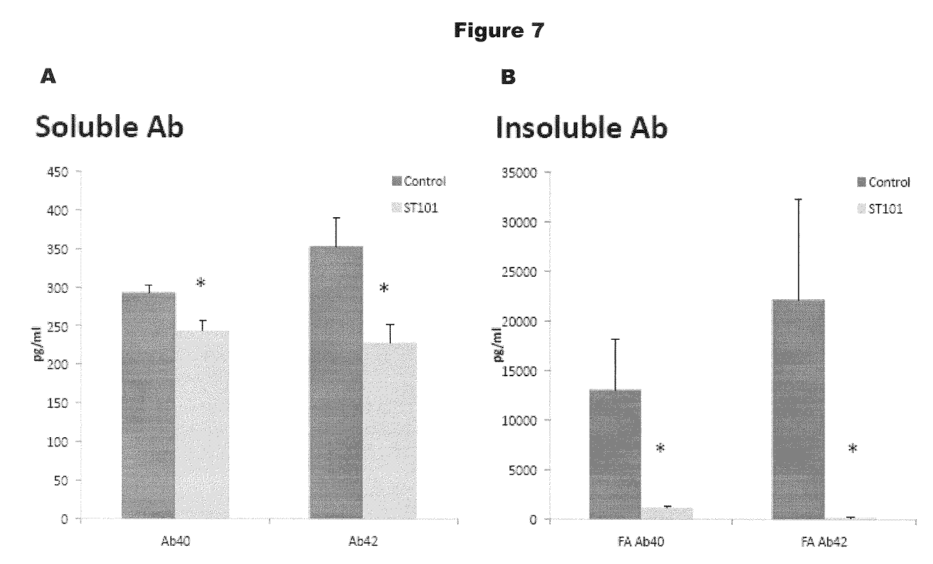

The previously described results of the Aβ-lowering effect of ST101 in 3xTg-AD mouse brain were obtained from approximately 12-month-old mice that had been treated with ST101 at 5/mg/kg/day in drinking water over a 2 month time period. Two experiments were conducted to confirm these findings. One experiment evaluated ST101 effects on 3×TG-AD mice of approximately 14.5 months of age after treatment for 2.5 months at the same dosage level (FIG. 7). FIGS. 7A and 7B are bar graphs that depict the effect of ST101 on Aβ in brain tissue from 3xTg-AD mice. FIG. 7A depicts the amounts of soluble Aβ₁₋₄₀ and Aβ₁₋₄₂ in brain tissue in mice treated with ST101, relative to control mice. FIG. 7B is a bar graph that depicts the amounts of insoluble Aβ₁₋₄₀ and Aβ₁₋₄₂ (formic acid extraction) in mice treated with ST101, relative to control mice. N=6/group. * denotes a statistically significant difference from control animals (p<0.05, Student's t-test). Group sizes: control n=6, ST101 n=6. Animals were approximately 14.5 months old at sacrifice after 2.5 months of treatment with ST101 at 5/mg/kg/day.

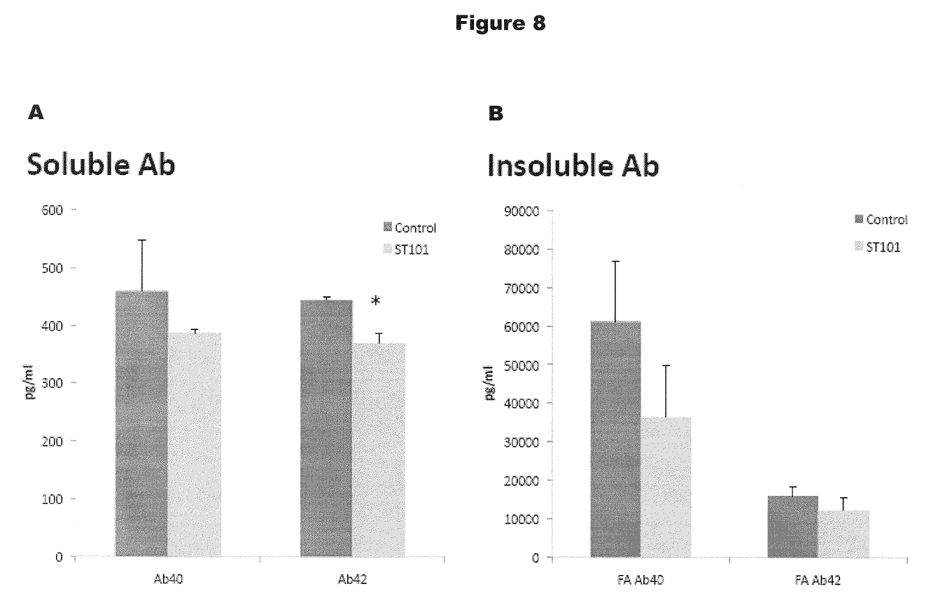

Another experiment evaluated ST101 effects in approximately 18 month-old animals after 2 months of treatment (FIG. 8). FIGS. 8A and 8B are bar graphs that depict the effect of ST101 on Aβ in brain tissue from 3xTg-AD mice. FIG. 8A depicts the amounts of soluble Aβ₁₋₄₀ and Aβ₁₋₄₂ in brain tissue in mice treated with ST101, relative to control mice. FIG. 8B a bar depicts the amounts of insoluble Aβ₁₋₄₀ and Aβ₁₋₄₂ (formic acid extraction) in mice treated with ST101, relative to control mice. * denotes statistically significant difference from control animals (p<0.05, Student's t-test). Group sizes: control n=4, ST101 n=6. Animals were approximately 20 months old at sacrifice after 2 months of treatment with ST101 at 5 mg/kg/day.

Both experiments show a reduction of Aβ₁₋₄₀ and Aβ₁₋₄₂ in “soluble” brain extracts as seen in the previous experiment. Reduction of Aβ in the “insoluble” fraction (obtained by formic acid extraction) was more variable. Previous data from 12 month-old mice showed no reduction of insoluble Aβ, whereas FIG. 7 showed a dramatic reduction of insoluble Aβ in 14.5 month-old mice that had underwent 2.5 instead of 2 months of treatment. 20-month-old mice showed a reduction of Aβ that did not reach statistical significance due to the small number of animals in the control group (n=4).

Overall, these results confirm a robust ST101 effect on levels of soluble Aβ. The effect was least pronounced in aged animals as these animals already have a large amyloid plaque burden before the onset of treatment. The greater variability of effects on Aβ in the insoluble fraction needs further follow-up. Effects due to animal age and length of treatment are possible, as well as technical issues with the effectiveness of formic acid extraction (less Aβ₁₋₄₂ was extracted from brains of the oldest mice).

### Example 8

### Effects of ST101 on Aβ Production In Vivo (Cynomolgus Monkeys)

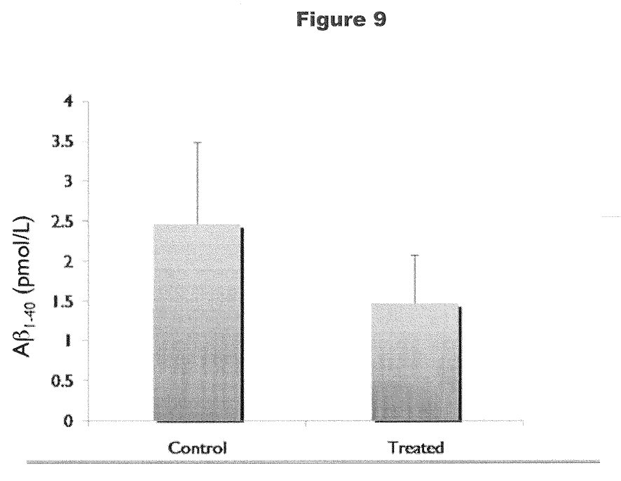

Brain samples from cynomolgus monkeys were obtained at the conclusion of a six-month chronic toxicity study. The cynomolgus monkeys used in this study were juveniles of less than 4 years of age. ST101 was administered daily for 6 months by naso-gastric tube at 10 mg/kg/day (n=8 per group). Data was generated from 8 treated and 8 control animals. Levels of Aβ₁₋₄₀ are shown in FIG. 9, which is a bar graph that depicts the effect of ST 101 on Aβ in brain tissue from cynomolgus monkeys. FIG. 9 depicts the amount of levels of Aβ₁₋₄₀ in monkeys treated with ST 101, relative to control monkeys.

Levels of Aβ in these juvenile animals were very low. There was a reduction of Aβ₁₋₄₀ in animals treated with ST101 that did not reach statistical significance. However, the mean Aβ₁₋₄₀ levels seen were at the lower limit of sensitivity of the assay and thus, Aβ₁₋₄₂ levels were not measurable.

The data indicate a reduction of Aβ₁₋₄₀ in cynomolgus monkey brain and support the data generated in the 3×TG-AD mice model. Further experiments are needed using monkey brain extracts to determine effects on APP processing using Western blots.

### Example 9

### Effect of ST101 on APP CTFs in Brain Tissue from 3xTg-AD Mice

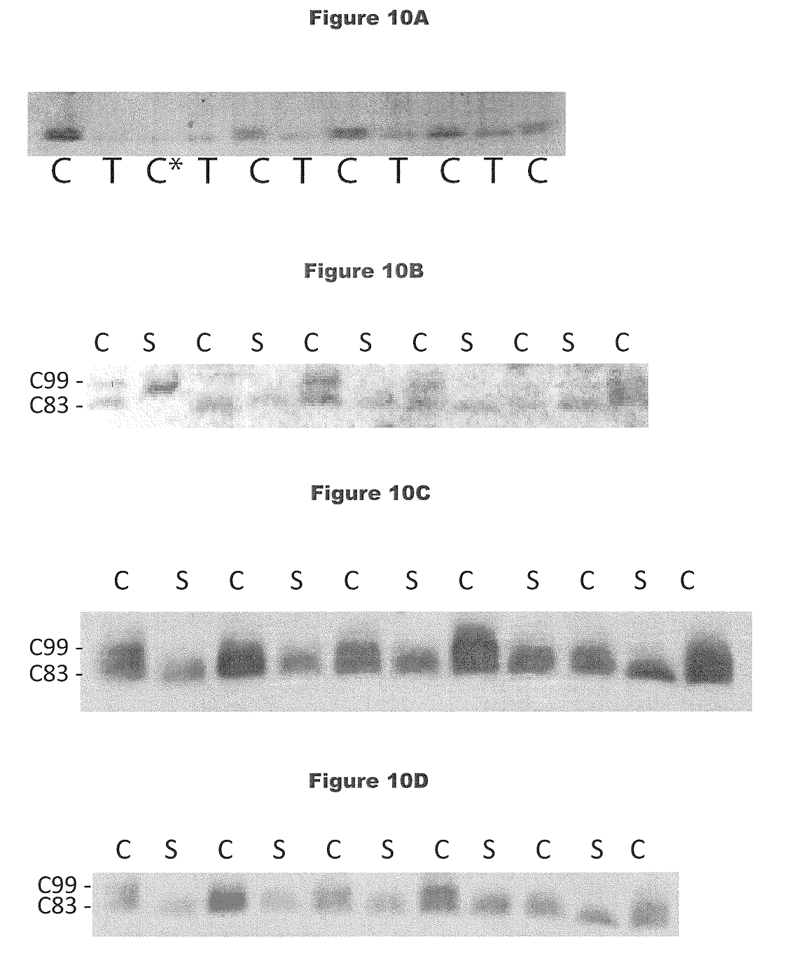

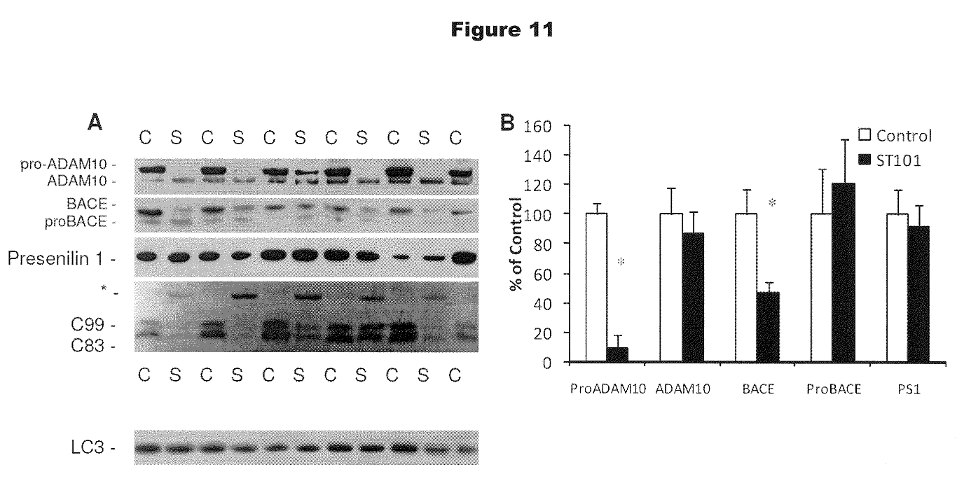

The previously described experiment in 12-month-old 3xTg-AD mice showed a profound reduction of C-terminal APP fragments. This effect was confirmed using brain extracts from 14.5 month-old 3xTg-AD mice treated for 2.5 months (FIGS. 10A-10B).

FIGS. 10A and 10B are Western blots that depict APP carboxy-terminal fragments detected by antibody CT20 in the brains of ST101-treated (T in FIG. 10A, S in FIG. 10B) 3xTg-AD mice, relative to untreated (C) 3xTg-AD mice. FIG. 10B is from a separate experiment that used the same brain extract used in the experiment for FIG. 10A. Animals were approximately 14.5 months old at sacrifice after 2.5 months of treatment with ST101 at 5 mg/kg/day in drinking water. * denotes a control animal with low levels of CTFs.

FIGS. 10C and 10D are Western blots that depict APP carboxy-terminal fragments detected by an APP C-terminal antibody (Eptitomics #: 15654-1) in the brains of ST101-treated (S) 3xTg-AD mice, relative to untreated (C) 3xTg-AD mice. FIG. 10D is a lighter exposure of the Western blot in FIG. 10C. Animals were approximately 14.5 months old at sacrifice after 2.5 months of treatment with ST101 at 5 mg/kg/day in drinking water.

The results in FIG. 10A confirm a significant effect of reduction of APP CTFs as seen in the earlier experiment (FIG. 4). However, the Western blot represented in FIG. 10A did not clearly resolve the C99 and C83 fragments. A repeat Western blot of the same brain extracts is shown in FIG. 10B. This Western blot achieved clear resolution of the C99 and C83 fragments. Although this particular Western blot exhibits some non-specific background it demonstrates that reduction of the C99 fragment is much more pronounced that reduction of the C83 fragment.

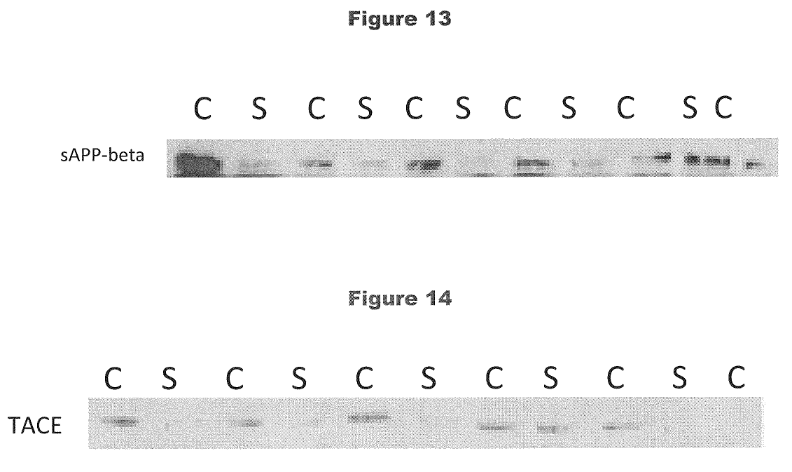

The results of FIGS. 10A and B were further confirmed in a repeat Western blot using a different C-terminal antibody directed against the C-terminal fragments of APP. The results of this repeat Western blot are shown as two different exposures of the same Western blot in FIGS. 10C and 10D. This Western blot confirms the reduction of the C99 fragment by ST101 treatment. It also confirms that the C83 fragment is not reduced to the same degree as in the previous experiment in 12-month old mice. The reduction of C99 can be explained by reduction of BACE as seen in the previous experiment (FIG. 11). Equivalent data on BACE are not yet available for the repeat experiment shown in FIG. 10A-D. The previous experiment had shown reduction of pro-ADAM-10, which explained the reduction of the C83 fragment (FIG. 11). In the experiment described here, C83 is not reduced to the same degree. This predicts a lesser effect of ST101 on ADAM-10 in this experiment. Data on ADAM-10 are not yet available from this experiment. A more selective effect of ST101 on BACE versus pro-ADAM10 is also consistent with a more pronounced reduction of A-beta (FIG. 7B) and a reduction of sAPP-beta (FIG. 13). The lesser of reduction of the C83 fragment is also consistent with the lack of detection of a 17 kDa in the experiment. As alpha-secretase (ADAM-10) cleavage seems largely intact in this experiment, there is no need for APP to be forced into an alternative pathway that creates the 17 kDa fragment. At this time it is not clear what determined the difference of the effect of ST101 on the C83 fragment between the two experiments in 12 month-old (FIG. 4) versus 14.5 month-old mice (FIGS. 10A-10D). However, the animals were of different age and treatment duration was different.

### Example 10

### Reduction of Beta-Secretase (BACE) and Pro-ADAM10 by Treatment with ST101 (3×TG-AD Mice)

APP C-terminal fragments C99 and C83 are created by beta-secretase and alpha-secretase cleavage respectively. The reduction of both C99 and C83 induced by ST101 could therefore be due to reduction or inhibition of secretases. This hypothesis was tested using Western blots of brain extracts from 3xTg-AD mice from the initial experiment in 12-month-old animals treated with ST101 for 2 months. The only beta-secretase is BACE1 and the constitutive alpha-secretase is ADAM10.

Western blots were probed with antibodies against BACE1 and ADAM10. The results are shown in FIG. 11. FIG. 11A is a series of Western blots depicting levels of proADAM10, ADAM10, proBACE, BACE, Presenilinl and APP-CFTs in brain extracts from ST101 treated 3×TG-AD mice (S) versus control mice (C). C: control brain extract. S: ST101-treated brain extract. Animals were approximately 12 months old at sacrifice after 2 months of treatment with ST101 at 5 mg/kg/day.

FIG. 11B depicts quantification of the Western blot bands from FIG. 11A by densitometry.

The Western blots show a profound reduction in BACE and pro-ADAM10. Pro-BACE and ADAM-10 protein levels are not significantly affected. These results are consistent with reduced activity of both alpha and beta-secretases leading to a reduction of APP-CTFs C99 and C83. Presenilin 1, a component of the gamma-secretase complex did not show significant changes.

Further experiments will include direct measurements of enzymatic activity of ADAM10 and BACE. These experiments will also address the questions raised by the differing effects of ST101 on pro-enzyme versus active enzyme levels: ST101 reduces BACE without reducing pro-BACE, but reduces pro-ADAM10 without reducing ADAM10.

### Example 11

### Reduction of Pathological Tau Accumulation In Vivo (3xTg-AD Mice)

The 3xTg-AD mouse model incorporates both pathological hallmarks of Alzheimer's disease: Aβ amyloid plaques and neurofibrillary tangles. Neurofibrillary tangles consist of accumulates of abnormally phosphorylated tau protein. In the 3xTg-AD mice pathological somato-dendritic accumulation of tau can be seen in immunohistochemistry as part of the disease model phenotype.

ST101 effects on tau distribution and accumulation were explored in immunohistochemistry with H7 anti-tau antibody. A clear reduction of pathological somato-dendritic tau staining was observed in the hippocampus. Hematoxylin/Eosin stained control sections showed no neuronal loss.

### Example 12

### Molecular Weight Shifts in Tau Species In Vivo (3xTg-AD Mice)

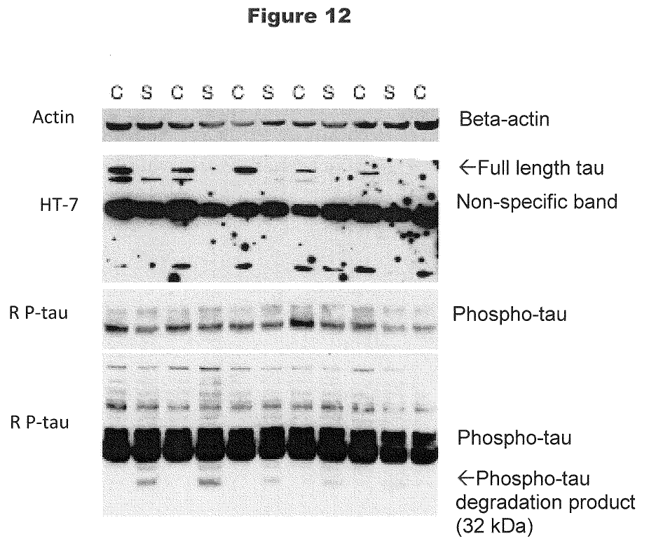

The status of tau phosphorylation, accumulation and degradation can be assessed in Western blots. Two anti-tau antibodies, directed against non-phosphorylated and phosphorylated tau, were used to probe brain extracts from the initial experiment. Western blots are shown in FIG. 12. FIG. 12 is a series of Western blots depicting levels of full-length tau, tau accumulates, tau degradation products and phosphorylated tau levels in brain extracts from ST101 treated 3×TG-AD mice (S) versus control mice (C). Beta actin levels (Ac) were used as a loading control. P-tau, two panels per antibody: top—normal exposure; bottom—overexposure to visualize minor protein bands. Ac: beta actin antibody as protein loading control. Animals were approximately 12 months old at sacrifice after 2 months of treatment with ST101 at 5 mg/kg/day.

Overexposed Western blots with both antibodies reveal subtle changes induced by ST101 treatment. H7 antibody showed the disappearance of accumulated tau and degradation products. R-p-tau antibody showed appearance of a new phosphorylated tau degradation product.

The discovery of reduction of BACE and proADAM10 provide major new insights in the mechanism of action of ST101 and provide new opportunities for screening assays to evaluate novel APPMs (amyloid processing pathway modulators).

Beyond BACE and pro-ADAM10, ST101 affects the concentration of other proteins as well. Effects of ST101 on TACE, huntingtin, and generalized effects on protein degradation are shown in the Examples below.

The data indicate that the potential disease-modifying effect of ST101 is mediated by reduction of BACE protein levels. The changes induced by ST101 represent a novel mechanism of BACE inhibition/reduction which does not seem to be shared with any known BACE inhibitors or BACE modulators. Reduction of BACE activity remains a major therapeutic goal in Alzheimer's disease.

ST101 also induced reduction of pro-ADAM10, the immediate precursor to the alpha secretase ADAM10, and decrease of products of alpha-secretase cleavage as reflected in lowered C83. On the surface, broad reduction of alpha-secretase activity could be considered an undesirable effect as alpha-secretases cleave multiple other substrates. However, ST101 has been proven safe in rodent and monkey toxicity studies of up to 6 months at doses up to approximately 100-fold higher than the ones used in the 3xTg-AD mice. This indicates that the levels of pro-ADAM10 reduction caused by ST101 are not sufficient to induce toxicity. This may be due to the specific nature of the effect of ST101 on pro-ADAM10 or due to activity of other alpha-secretases compensating for a major effect of ST101 on pro-ADAM10.

Further work will have to address the specificity of the effects of ST101 for BACE and pro-ADAM10. It is likely that ST101 will affect other proteins as well. In this context, it will be interesting to determine whether ST101 affects ADAM17, another alpha-secretase, also known as TACE, tumor necrosis factor converting enzyme. If ST101 reduces levels of TACE, this would open new opportunities for use of ST101 as a TNF,modulator.

The presented experiments confirm a profound effect of ST101 on APP processing and A-beta production Demonstration of downregulation of BACE and ADAM10 provide a plausible explanation of ST101 effects on A-beta production.

### Example 13

### Effect of ST101 on sAPP-Beta in Brain Tissue from 3xTgAD Mice

FIG. 10B describes a reduction of fragment C99. C99 is created by BACE cleavage which leads to release of soluble APP, specifically sAPP-beta. Thus, a reduction of C99 predicts a concomitant reduction of sAPP-beta. This was tested by Western blot as shown in FIG. 13. The Western blot was obtained using an sAPP-beta specific antibody in brain extracts from ST101 treated 3×TG-AD mice (S) versus control mice (C). Animals were approximately 14.5 months old at sacrifice after 2 months of treatment with ST101 at 5 mg/kg/day in drinking water.

FIG. 13 confirms that the reduction of C99 in FIG. 10B was accompanied by a concomitant reduction of sAPP-beta. In the previous experiment that also showed a significant reduction of C99, sAPP-beta could not be detected due to technical difficulties.

### Example 14

### Effect of ST101 on TACE in Brain Tissue from 3xTgAD Mice

In addition to ADAM10, ADAM17 (TACE, which is Tumor Necrosis Factor converting enzyme) is known to act as an alpha-secretase of APP. Thus, it was tested in a Western blot whether ST101 reduces levels of ADAM17 (TACE).

FIG. 14 is a Western blot that shows the effect of ST101 on TACE in brain tissue from 3xTgAD Mice. The Western blot was obtained using a TACE specific antibody in brain extracts from ST101 treated 3×TG-AD mice (S) versus control mice (C). Animals were approximately 14.5 months old at sacrifice after 2 months of treatment with ST101 at 5 mg/kg/day in drinking water.

FIG. 14 shows a reduction of TACE levels in the majority of ST101 treated animals, compared to control animals. This suggests that ST101 is capable of reducing TACE levels.

The breadth and scope of the present invention should not be limited by any of the above-described exemplary embodiments, but should be defined only in accordance with the following claims and their equivalents.

### Example 15

### Effect of ST101 on Levels of Ubiquitylated Proteins

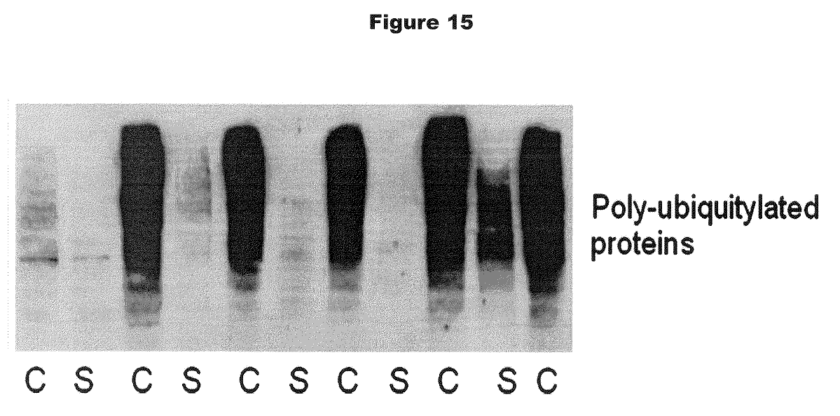

Brain extracts from 12-month-old 3xTg-AD mice that had been treated with ST101 (S) at 5/mg/kg/day in drinking water over 2 months were analyzed in Western blots using an anti-ubiquitin antibody (Dako no. Z0448). The results are shown in FIG. 15, which illustrates a profound reduction of ubiquitylated proteins. The control (C) samples show a typical high molecular weight “smear” representing a mixture of ubiquitylated proteins. The level of these proteins was significantly reduced after treatment with ST101. Of note, monomeric ubiquitin concentrations were largely unaffected by ST101 treatment (data not shown).

Confirmatory experiments in samples from 14.5-month-old mice, treated for approximately 2.5 months with 5 mg/kg/day of ST101, were conducted and also showed a significant reduction of ubiquitylated proteins in brain extracts from ST101-treated animals, compared to control animals.

### Example 16

### Effect of ST101 on Ubiquitylated α-Tubulin

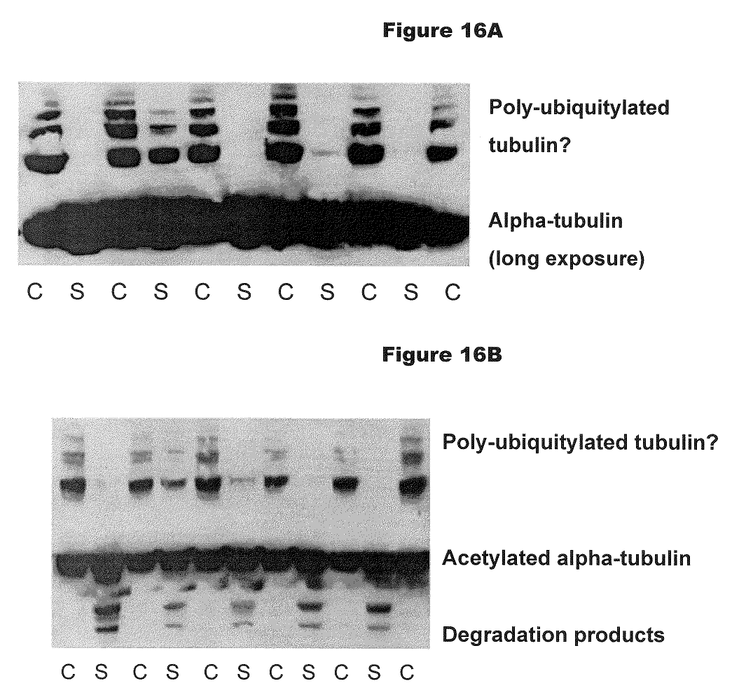

Probing of the same samples as in Example 15 in Western blots revealed the patterns shown in FIGS. 16A (which employed an α-tubulin antibody) and 16B (which employed acetylated α-tubulin antibody).

FIG. 16A shows a long-exposure Western blot. The overexposed band near the bottom of the figure represents α-tubulin. Above this band a ladder of immuno-reactive bands was detected by the antibody. This ladder was either absent or significantly reduced in samples from ST101-treated (S) animals, versus control (C) animals.

FIG. 16B, confirms this finding and, in addition, demonstrates the presence of reduced tubulin degradation products after treatment with ST101.

The molecular weight shift represented in the high molecular weight ladder was consistent with the addition of one or several ubiquitin molecules to α-tubulin. At this point, though an unproven hypothesis that needs to be followed-up experimentally, it appears obvious that ST101 had a major effect in reducing the high molecular weight ladder.

### Example 17

### Effect of ST101 on Huntingtin Protein

The data provided herein suggests that enhanced UPS degradation may be the mechanism by which treatment of ST101 leads to marked reduction of BACE, pro-ADAM10 and tau protein. However, the effect of ST101 on ubiquitylation, shown above, also suggests that if the actions of ST101 are directed toward the UPS, ST101 should facilitate removal of many other proteins degraded by this mechanism.

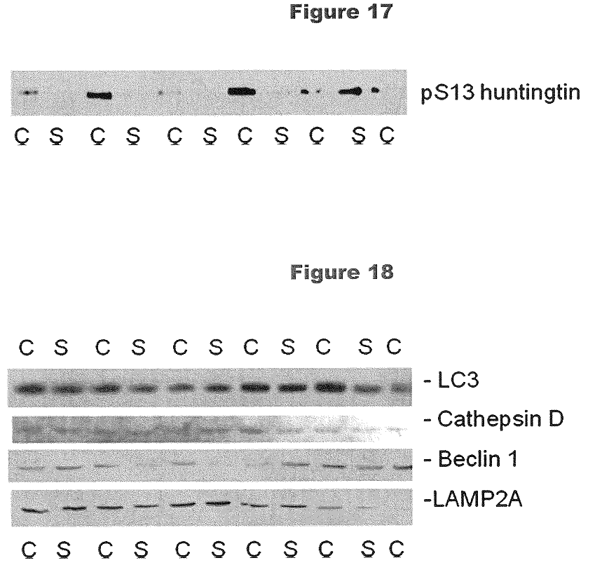

In a first pilot experiment to explore the effect of ST101 on proteins that accumulate in neurodegenerative diseases other than AD, brain extracts from ST101-treated 3xTg-AD mice (S) and control mice (C) mice were analyzed by Western blotting with an antibody directed against huntingtin, specifically against a phospho-epitope around serine 13 (pS13 huntingtin). The result is shown in FIG. 17. These extracts are from the same mice as those used in Examples 15 and 16.

Protein levels of huntingtin were significantly reduced in ST101 treated animals (S) compared to control animals (C) in the majority of cases.

### Example 18

### Effect of ST101 on Mediators of the Autophagy and Lysosomal Degradation Pathways

A number of studies have implicated defects of autophagy and lysosomal degradation in Alzheimer's disease (for review, see Shacka et al., Front Biosci. 13: 718-36 (2008)). Particular observations that support the role of these pathways in amyloidogenesis include the presence of lysosomal targeting sequences in APP and BACE (Koh et al., J. Biol. Chem. 280(37): 32499-504 (2005), Thinakaran et al., J. Biol. Chem. 283(44):29615-9 (2008)).

The concentrations of several proteins involved in these pathways, specifically LC3, Beclin 1 (macroautophagy), LAMP2A (chaperone mediated autophagy), and cathepsin D (lysosomal protease) were measured in Western blots from 3xTg-AD mouse brains (FIG. 18, extracts from the same animals used in Examples 15-17.

The Western blot analysis in FIG. 18 did not reveal any obvious differences between ST101-treated (S) and control (C) animals, suggesting that macroautophagic pathways, the regulation of LAMP2A, and the activity of cathepsin D may not be determinants of ST 101's ability to decrease ubiquitylated proteins.

In addition to the reduction of APP processing and degradation products (sAPP beta, C99, C83, Aβ₁₋₄₀, Aβ₁₋₄₂), ST101 has shown significant reductions of tau protein, pro-ADAM10, BACE1, Huntingin, and α-tubulin high molecular weight ladders.

The reduction of ubiquitylated proteins provides a common mechanism explaining the effect of ST101 on a diverse group of proteins, but not all proteins. Ubiquitylated proteins are targeted for degradation by both the lysosome and the proteasome. Literature reports indicate that tau, BACE, huntingtin and tubulin are subject to UPS degradation. (Babu et al., J. Neurochem. 94:192-203 (2005); Quing et al., FASEB J. 18:1571-3 (2004); Yang et al., Exp Cell Res. 313(3):538-50 (2007); and Ren et al., J. Neurosci. 23(8):3316-24 (2003)). No specific reports were found on ADAM-10 degradation, but other related “sheddases” are ubiquitylated.

ST101 reduced the amount of ubiquitylated proteins without significant change in levels of free ubiquitin. This suggests that ST101 may increase degradation of the ubiquitylated proteins by the lysosome and/or the proteasome. An obvious next step for experimental follow-up will be direct measurements of proteasome function after treatment with ST101.

An alternative explanation for reduced ubiquitylated proteins would be inhibition of ubiquitylation activity. However, this does not fit with the other available data, because (1) the end result should be increased rather than decreased levels of tau, BACE ADAM10 etc.; (2) free ubiquitin levels should be increased instead of unaltered; and (3) inhibition of the UPS should be toxic. For instance, proteasome inhibitors such as Velcade are very toxic. In contrast, ST101 has shown very little toxicity in chronic toxicity studies at high doses and in reproductive toxicity studies where perturbation of this system has serious developmental consequences.

ST101 is the first small molecule drug ever discovered to produce a dramatic reduction in the concentrations of ubiquitylated proteins combined with alterations in the processing of multiple proteins and improvement in learning and memory. The discovery described herein demonstrates that small molecules can enhance protein degradation, in particular through the ubiquitin proteasome pathway. Thus, it is expected that the compounds described herein are useful for treatment of a wide variety of diseases that will benefit from reductions in ubiquitylated proteins.

These data taken together with the findings described herein suggest an indirect mechanism for the reduction of Aβ and sAPP-β: ST101 leads to the reduction of BACE and reduced BACE levels lead to reduced production of Aβ.

However, it cannot be ruled out that in addition, ubiquitylation of Aβ can occur, in particular as the presence of ubiquitin has been demonstrated in Aβ plaques.

The ubiquitylation system is very complex, with multiple ubiquitin ligases and their activating and conjugating enzymes. It is not clear to date whether the molecular target of ST101 is at the level of ubiquitylation or upstream or downstream at the lysosome or proteasome level. Further work is required to confirm whether ST101 acts as a general enhancer of lysosome or proteasome function, whether it enhances protein degradation of a more selective subset of proteins or whether it also enhances protein degradation via other pathways. However, it is clear that the apparent effect of ST101 in accelerating the ubiquitin/proteasome pathway is an unprecedented pharmacological activity not previously observed with any small molecule drug.

The effects of ST101 on the UPS can be linked to its effects in models of learning and memory. ST101 has demonstrated efficacy in a multitude of models of learning and memory (Yamaguchi et al., J Pharmacol Exp Ther. 317(3):1079-87 (2006); Ito et al., J. Pharmacol. Exp. Ther. 320(2): 819-27 (2007)), as well as efficacy in increasing long-term potentiation (LTP) (Han et al., J. Pharmacol. Exp. Ther. 326(1): 127-34 (2008)). These studies included learning and memory defects induced by direct Aβ administration. A recent publication describes that administration of the ubiquitin hydrolase Uch-L1 reverses Aβ-induced decrease in synaptic function in hippocampal slices and improves learning and memory in a mouse model of AD (Gong et al., Cell. 126(4): 775-88 (2006)). These data link functional improvements to improved function of the UPS. ST101 increases clearance of ubiquitylated proteins. This mechanism provides an explanation for functional improvements in at least some of the models described by Yamaguchi et al., Ito et al. and Han et al. ST101 can be used to treat or prevent other diseases. Increased or accelerated clearance of ubiquitylated proteins induced by ST101 may be useful not only in AD, but in a number other diseases (for review see: Dahlmann et al., 2007). A number of diseases are characterized by accumulation of misfolded proteins, toxic proteins or toxic degradation products, many of which accumulate in inclusion bodies or as extracelluar deposits: Alzheimer's disease: Aβ-plaques and tau neurofibrillary tangles; Parkinson's disease (Lewy bodies with α-synuclein), Huntington's disease (aggregates of huntingtin) and prion disease (misfolded PrPᵥₐᵣᵢₐₙₜ). In all these diseases the UPS has been implicated (Ross et al., Trends Cell Biol. 14(12): 703-11 (2004); Bennett et al., Nature 448(7154): 704-8 (2007); Lauren et al., Nature 457(7233): 1128-32 (2009)).

Increased degradation of ubiquitylated proteins induced by ST101 may have use in many other diseases, for instance cataracts of the lens, type 2 diabetes and Paget's disease of the bone. Accumulation of misfolded crystallins is responsible for the generation of age-related cataracts of the lens (Stiuso et al., FEBS Lett. 531(2): 162-7 (2002)). Islet amyloid polypeptide (IAPP) forms amyloid in β-cells of the pancreas (Huang et al., Am J Physiol Endocrinol Metab. 293(6): E1656-62 (2007)). Mutations of the ubiquitin associated domain of the protein p62 lead to Paget's disease of the bone characterized by reduced osteoclast function (Layfield et al., Biochem Soc Trans. 36(Pt 3): 469-71 (2008)).

ST101 may also provide benefit in diseases with increased protein turnover. For instance in stroke or trauma, in particular traumatic brain injury, massive amounts of proteins from damaged cells have to be removed and degraded. In the case of traumatic brain injury APP is upregulated during the repair process, leading to increased production of Aβ (Loane et al., 2009). Traumatic brain injury is a known risk factor for the development of Alzheimer's disease (Dementia pugilistica). Thus, ST101 may prove beneficial in two ways: it may increase the removal of damaged and/or defective proteins and it may prevent the accumulation of harmful byproducts generated during the protein processing.

Decreased proteasomal activity is believed to play a role in the normal aging process. ST101 has been tested in a model of accelerated senescence (SAM-P8 mice). In two independent experiments with 16 and 32 weeks of treatment, ST101-treated mice showed a significant improvement of senescence scores in comparison to control mice. These findings are shown in FIG. 5 of International Patent Application No. PCT/JP2007/070963, which published as WO 2008/047952. The data in WO 2008/047952 indicate that ST101 significantly reduced the senescence score in SAM-P8 mice. An ST101 induced increase of clearance of proteins by the ubiquitin-proteasome system provides a likely explanation for the improved senescence scores.

ST101 dramatically reduces the amount of ubiquitylated proteins likely by making breakdown of accumulated and/or damaged proteins more efficient. This discovery provides an explanation for the reduction of BACE and Aβ by ST101 and is a likely basis for ST101's efficacy in mouse models of AD. This mechanism is not specific for AD. Thus, ST101 has the potential for therapeutic benefit in many other diseases in which the UPS is involved. It has been already demonstrated that ST101 reduces the concentrations of huntingtin protein, albeit in a non-Huntington's Disease model. Therefore, ST101 may have therapeutic benefit in Huntington's disease. Other disease targets include neurodegenerative diseases such as Parkinsons' Disease and prion disease, stroke, traumatic brain injury as well as non-CNS diseases such as type 2 diabetes, cataracts, Paget's disease of the bone and many others. ST101 may also have effects on the normal ageing process.

ST101 is the first small molecule drug ever discovered to produce a dramatic reduction in the concentrations of ubiquitylated proteins, related to enhanced activity of the Ubiquitin Proteasome System, that is coupled to equally dramatic alterations in protein processing pathways and improvement in learning and memory. The discovery demonstrates that these activities represent new targets for the treatment of disease with small molecule drugs.

### Example 19

### Effect of ST101 on App Processing and A-Beta Production in Brain Tissue from Cynomolgus Monkeys

The previously described examples showed an effect of ST101 in 3xTg-AD mice and in juvenile normal cynomolgus monkeys at the end of a 6-month toxicity study. Such effects were confirmed using brain samples from cynomolgus monkeys obtained at the conclusion of a 9-month chronic toxicity study.

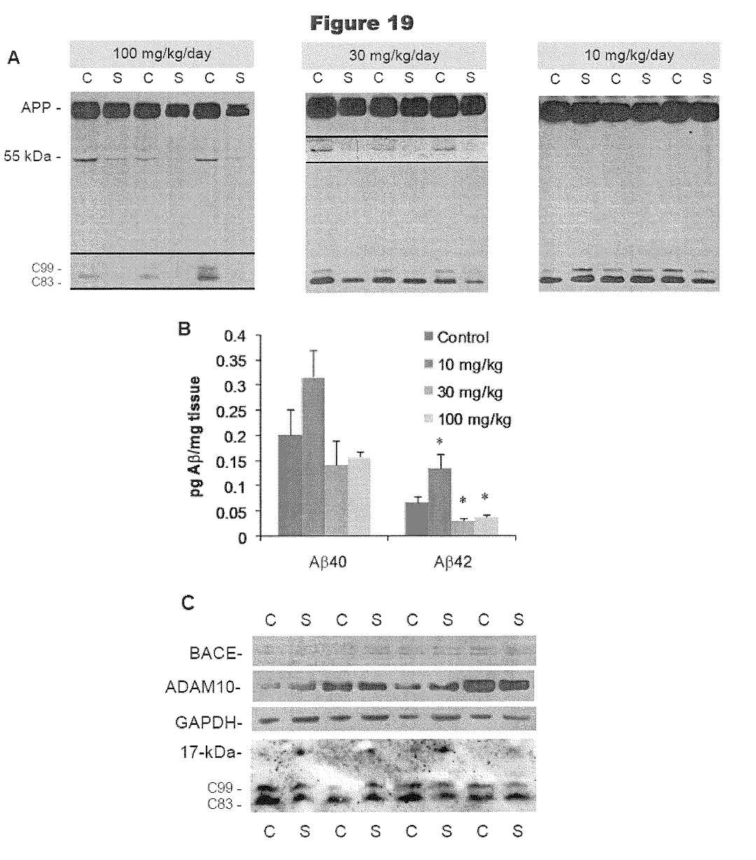

Male cynomolgus monkeys were treated for 9 months with 0, 10, 30 or 100 mg/kg/day ST101 with once daily oral drug administration. Hippocampal brain samples were analyzed in Western blots and ELISAs for APP fragments and Aβ. FIG. 19A: Western blot analyses of APP with a C-terminal APP antibody 1565-1 (Epitomics) revealed reductions in both C83 and C99 in the 30 and 100 mg/kg/day groups, but no differences in the 10 mg/kg/day group. Reductions in a ˜55-kDa fragment were also seen with treatment in these groups. Black outlines depict image level enhancement in Adobe Photoshop CS4, to better visualize weak signals. FIG. 19B: Sandwich ELISA measurements of hippocampal lysates revealed significant increases in Aβ42 in the 10 mg/kg/day ST101 group, but significant decreases in both the 30 and 100 mg/kg/day ST101 groups. FIG. 19C: Western blot analyses of hippocampal brain samples show no differences in the 100 mg/kg/day group for either BACE or ADAM10. However, long exposures reveal the presence of the 17-kDa APP fragment only in treated monkeys (antibody CT20, Calbiochem). This fragment was not detected in either the 30 or 10 mg/kg/day ST101 groups. Error bars indicate SEM (standard error of the mean) and * indicates statistical significance versus control (p<0.05). (S: ST101-treated animals, versus C: control animals).

These findings in cynomolgus monkeys confirm ST101 effects seen in 3xTg-AD mice on APP fragments and A-beta. This indicates that ST101 has activity in primates and has the ability to penetrate into the primate brain in order to exert the described effects.

There were some differences in ST101 effects between 3xTg-AD mice and cynomolgus monkeys. In particular no effect was detected on BACE and ADAM-10 in monkeys in contrast to the 3xTg-AD mice. These differences may be due to species differences in metabolism, differences in brain penetration, juvenile status of the normal cynomolgus monkeys versus the adult status of transgenic mice, low expression levels of APP and A-beta in the monkeys versus high levels in the transgenic mice, differences in ST101 doses, differences in brain regions (cortex in mice versus hippocampus in monkeys), or differences in dose administration (mice: continuous administration in drinking water; monkeys once daily administration by oral gavage).

The lack of an ST101 effect on BACE and ADAM-10 seen in this monkey experiment is in contrast to the effect seen in transgenic mice (Example 10). This indicates that an effect on BACE and ADAM-10 may not be necessary for ST101 to lower the production of A-beta. Rather, the more general effects of ST101 on protein ubiquitylation and protein degradation described in Examples 15, 16 and others may be the primary cause for altered APP processing and decreased A-beta production.

### Example 20

Effect of ST101 on Acetylated Alpha-Tubulin and Beta-Tubulin in Brain Tissue from Cynomolgus Monkeys

Example 16 shows an effect of ST101 on alpha-tubulin ubiquitylation and on acetylated alpha-tubulin degradation in 3xTg-AD mice. The effects on tubulin were confirmed in the group of cynomolgus monkeys described in example 19.

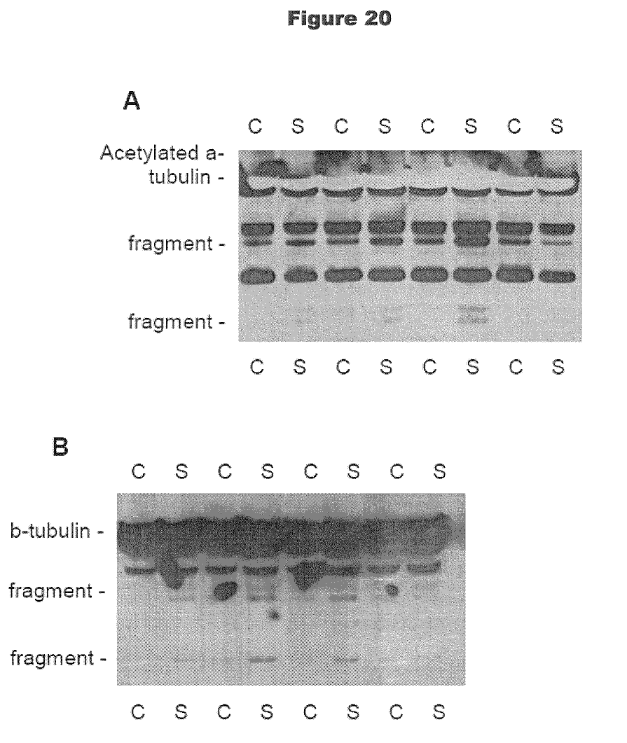

Western blots of hippocampal brain samples revealed the patterns shown in FIGS. 20A (which employed an acetylated α-tubulin antibody) and 20B (which employed beta-tubulin antibody).

FIG. 20A shows a reduction of acetylated alpha-tubulin fragments in ST101-treated (S) animals, versus control (C) animals. FIG. 20B shows an increase of beta-tubulin degradation products after treatment with ST101.

These data indicate that ST101 has general effects on protein degradation in both mice and cynomolgus monkeys.

### Example 21

### Effect of ST101 on Levels of Ubiquitylated Proteins in Normal Mice

In order to detect whether the effect of ST101 on protein ubiquitylation seen in 3xTg-AD mice as shown in Example 15 could also be seen in normal mice, C57/B6 wildtype mice were treated with ST101.

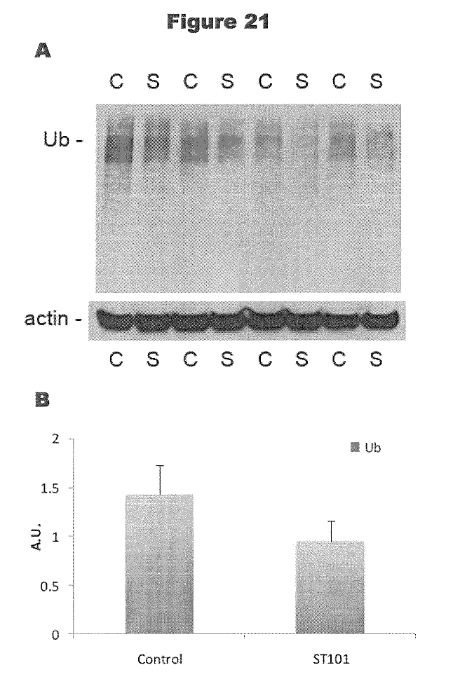

Brain extracts from 2 month-old C57/B6 wildtype mice that had been treated with ST101 at 5/mg/kg/day in drinking water over 2 months were analyzed in Western blots using an anti-ubiquitin antibody (Dako no. Z0448). The results are shown in FIG. 21 A, which illustrates a modest reduction of ubiquitylated proteins comparing control (C) brain samples with ST101 treated samples (S). FIG. 21B shows quantification of the Western blot signal from FIG. 21A by densitometry (mean+/−SEM) in arbitrary units after normalization for the beta-actin signal (loading control).

### Example 22

### Effect of ST101 on Levels of Proteasome Subunits

The ST101 effect of reducing ubiquitylated proteins may be due to increased proteasome activity accelerating the clearance of ubiquitylated proteins. Thus, one possible mechanism of action of ST101 is a direct effect on the assembly or composition of the proteasome, which consists of multiple subunits.

Brain extracts from 12-month-old 3xTg-AD mice (described in Example 15) that had been treated with ST101 at 5/mg/kg/day in drinking water over 2 months were analyzed in Western blots using an anti-proteasome alpha-subunit antibody.

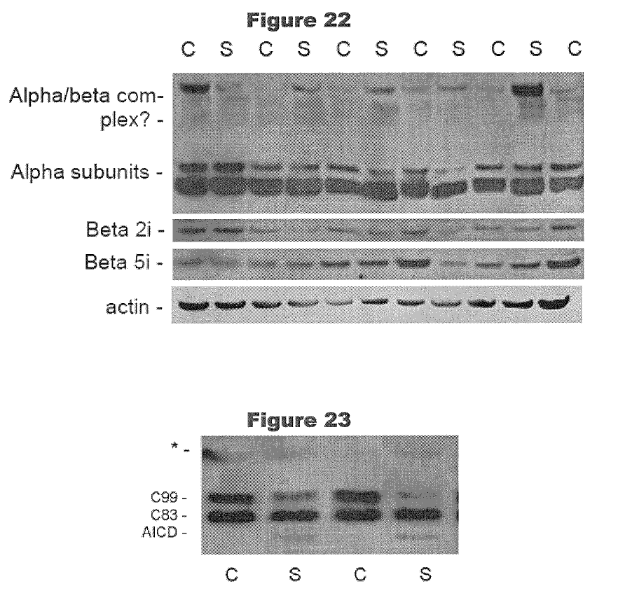

FIG. 22 shows proteasome alpha subunits in brain extracts from ST101-treated (S) and control animals (C). No difference is apparent in the concentration of proteasome alpha subunits. However, there is a clear decrease in high molecular weight immuno-reactive bands after ST101 treatment. As these bands react with the proteasome alpha-subunit antibody they may represent complexes of alpha-subunits with other subunits. Antibodies against two other proteasome subunits (beta 2i and beta 5i) did not reveal changes after ST101 treatment.

### Example 23

### Effect of ST101 on the APP-AICD Fragment

The processing of APP by gamma-secretase leads to the generation of an additional C-terminal fragment termed the AICD fragment (APP intracellular domain, shown schematically in FIG. 6). Once generated by gamma-secretase, the AICD fragment can be transported to the nucleus where it acts as a transcriptional activator. Although the biological role of AICD-mediated transactivation is not clear, it has been proposed that suppression of its production by gamma-secretase inhibitors may have adverse effects. Thus, it was examined whether ST101 affects the production of the AICD fragment.

Brain extracts from 12-month-old 3xTg-AD mice that had been treated with ST101 (S) at 5/mg/kg/day in drinking water over 2 months were analyzed in Western blots using a C-terminal APP antibody (CT-20 Calbiochem). The results are shown in FIG. 23. The AICD fragment is visible in brain extracts from ST101 treated mice (S) but not in control mice (C).

These data indicate that production of the AICD fragment is not reduced, but rather increased by treatment with ST101.

What is claimed is:

1. A method of decreasing the level of ubiquitylated protein in a human subject, the method comprising administering to a subject in need thereof an effective amount of a heterocyclic compound having the general Formula (I): 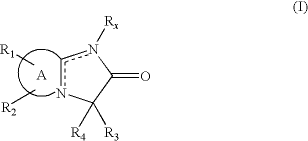
or a pharmaceutically acceptable salt, hydrate or prodrug thereof to a human subject in need thereof, wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 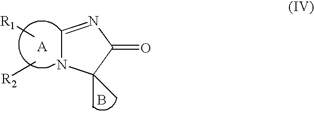
wherein B may be one or more structural units selected from structural units having the general Formula (V), 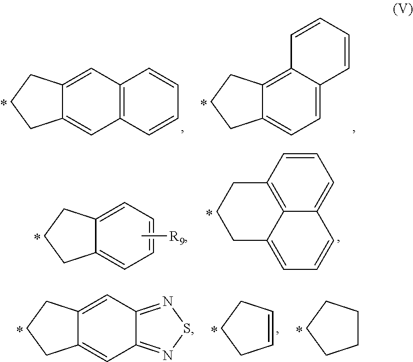
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

2. A method of enhancing the removal and degradation of harmful proteins in a human subject, the method comprising administering to a subject in need thereof an effective amount of a heterocyclic compound having the general Formula (I): 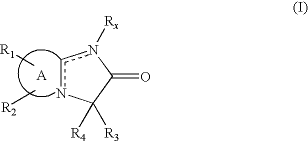
or a pharmaceutically acceptable salt, hydrate or prodrug thereof to a human subject in need thereof, wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 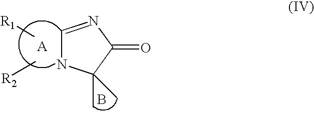
wherein B may be one or more structural units selected from structural units having the general Formula (V), 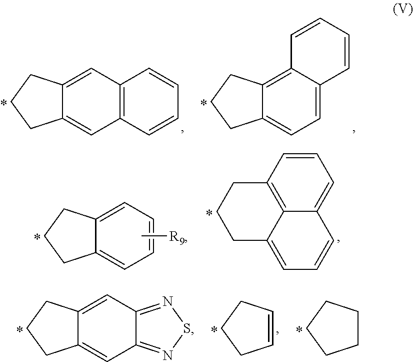
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

3. A method of enhancing proteasome breakdown of accumulated and/or harmful protein in a human subject, the method comprising administering to a subject in need thereof an effective amount of a heterocyclic compound having the general Formula (I): 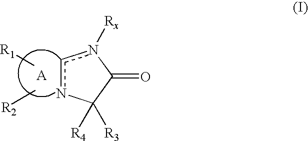
or a pharmaceutically acceptable salt, hydrate or prodrug thereof to a subject in need thereof,
wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 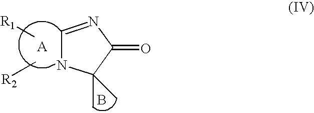
wherein B may be one or more structural units selected from structural units having the general Formula (V), 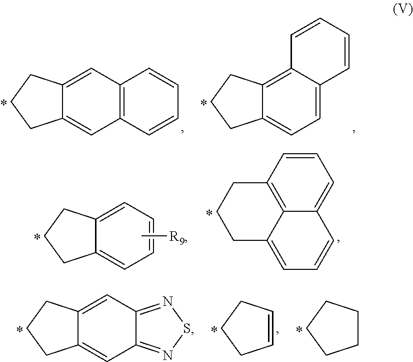
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

4. A method of enhancing the activity of the ubiquitin-proteasome system pathway in a human subject, the method comprising administering to a subject in need thereof an effective amount of a heterocyclic compound having the general Formula (I): 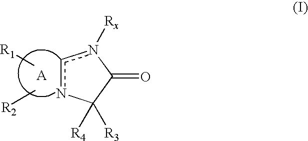
or a pharmaceutically acceptable salt, hydrate or prodrug thereof to a human subject in need thereof,
wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 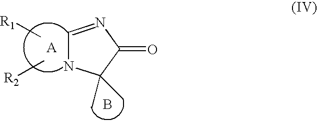
wherein B may be one or more structural units selected from structural units having the general Formula (V), 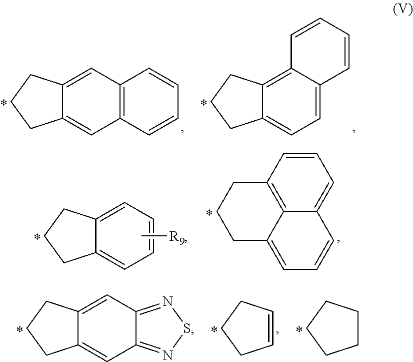
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

5. A method of treating or preventing cardiac dysfunction, the method comprising administering to a subject in need thereof an effective amount of a heterocyclic compound having the general Formula (I): 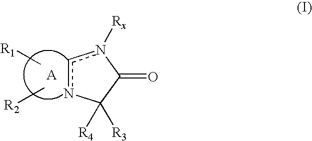
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 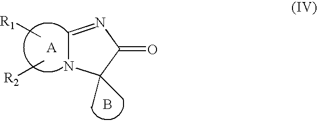
wherein B may be one or more structural units selected from structural units having the general Formula (V), 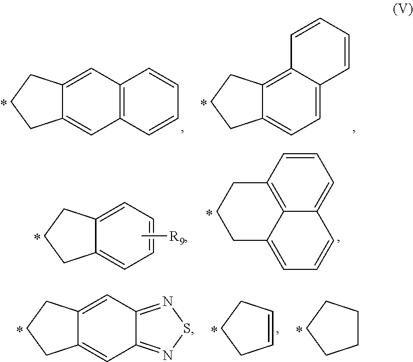
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

6. A method of treating or preventing spinobulbar muscular atrophy (Kennedy Disease), the method comprising administering to a subject in need thereof an effective amount of a heterocyclic compound having the general Formula (I): 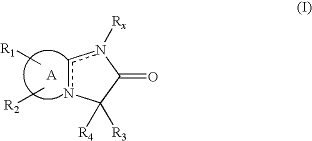
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 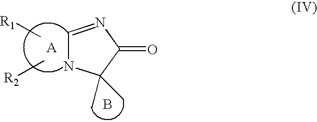
wherein B may be one or more structural units selected from structural units having the general Formula (V), 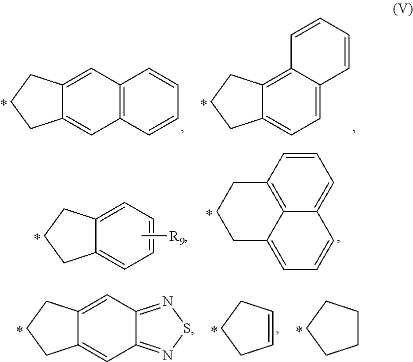
the structural unit B binds at a position marked by in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

7. A method of treating or preventing dentatorubral-pallidoluysian atrophy (Haw River Syndrome), the method comprising administering to a subject in need thereof an effective amount of a heterocyclic compound having the general Formula (I): 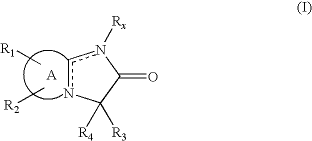
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 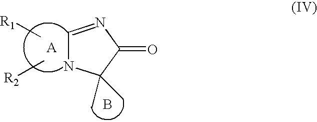
wherein B may be one or more structural units selected from structural units having the general Formula (V), 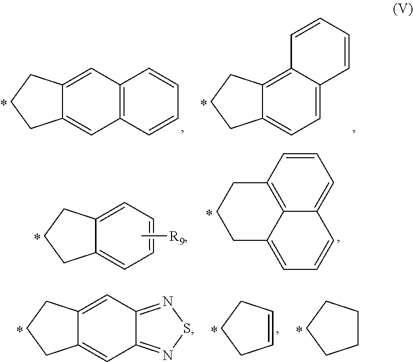
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

8. A method of treating or preventing spinocerebellar ataxia, the method comprising administering to a subject in need thereof an effective amount of a heterocyclic compound having the general Formula (I): 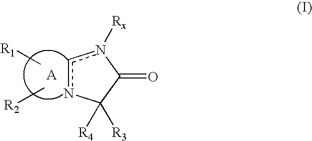
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 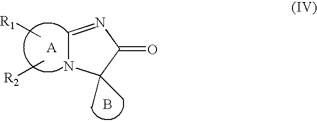
wherein B may be one or more structural units selected from structural units having the general Formula (V), 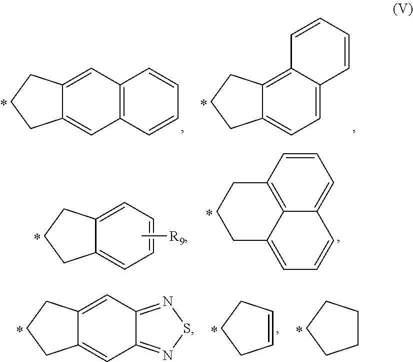
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

9. A method of treating or preventing macular degeneration, the method comprising administering to a subject in need thereof an effective amount of a heterocyclic compound having the general Formula (I): 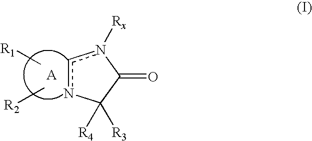
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 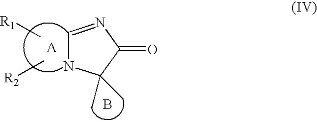
wherein B may be one or more structural units selected from structural units having the general Formula (V), 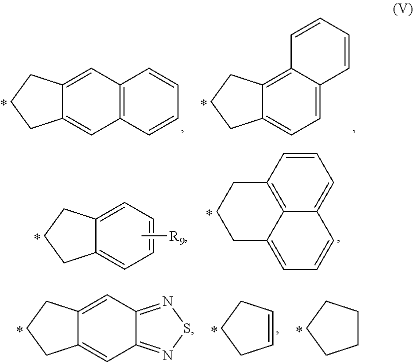
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

10. A method of decreasing the level of ubiquitylated protein in a human subject, the method comprising administering to a subject in need thereof an effective amount of a compound that is not a heterocyclic compound having the general Formula (I): 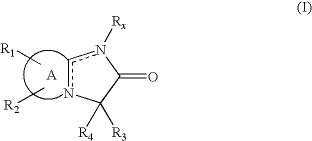
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 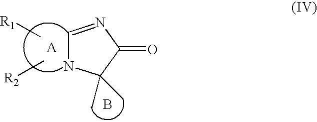
wherein B may be one or more structural units selected from structural units having the general Formula (V), 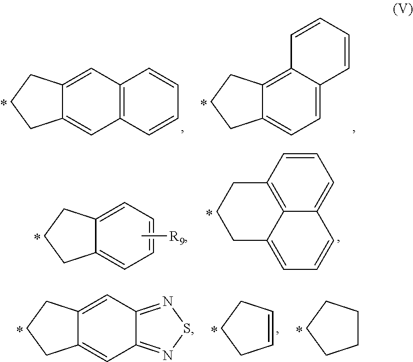
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group, and
wherein the compound is not a ubiquitin hydrolase.

11. A method of enhancing the removal and degradation of harmful proteins in a human subject, the method comprising administering to a subject in need thereof an effective amount of a compound that is not a heterocyclic compound having the general Formula (I): 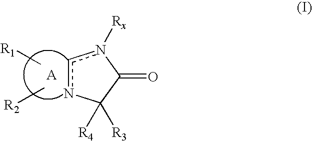
or a pharmaceutically acceptable salt, hydrate or prodrug thereof to a human subject in need thereof,
wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 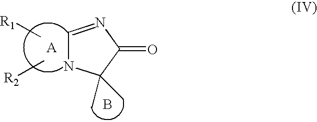
wherein B may be one or more structural units selected from structural units having the general Formula (V), 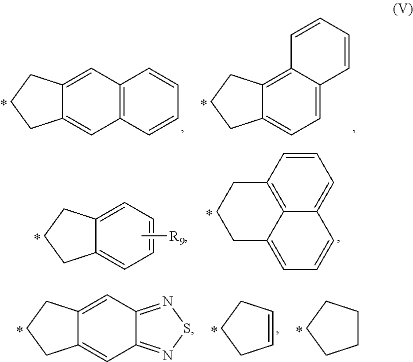
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group, and
wherein the compound is not a ubiquitin hydrolase.

12. A method of enhancing proteasome breakdown of accumulated and/or harmful protein in a human subject, the method comprising administering to a subject in need thereof an effective amount of a compound that is not a heterocyclic compound having the general Formula (I): 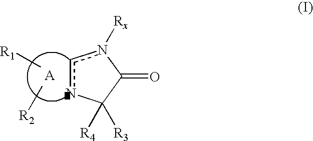
or a pharmaceutically acceptable salt, hydrate or prodrug thereof to a human subject in need thereof,
wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the groupconsisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 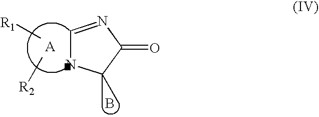
wherein B may be one or more structural units selected from structural units having the general Formula (V), 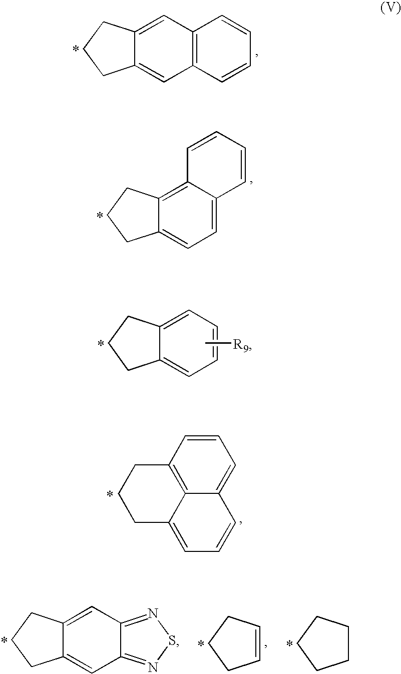
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group, and
wherein the compound is not a ubiquitin hydrolase.

13. A method of enhancing the activity of the ubiquitin-proteasome system pathway in a human subject, the method comprising administering to a subject in need thereof an effective amount of a compound that is not a heterocyclic compound having the general Formula (I): 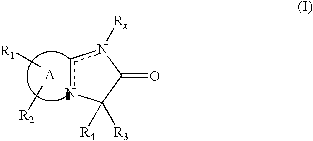
or a pharmaceutically acceptable salt, hydrate or prodrug thereof to a human subject in need thereof,
wherein
Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 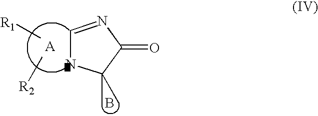
wherein B may be one or more structural units selected from structural units having the general Formula (V), 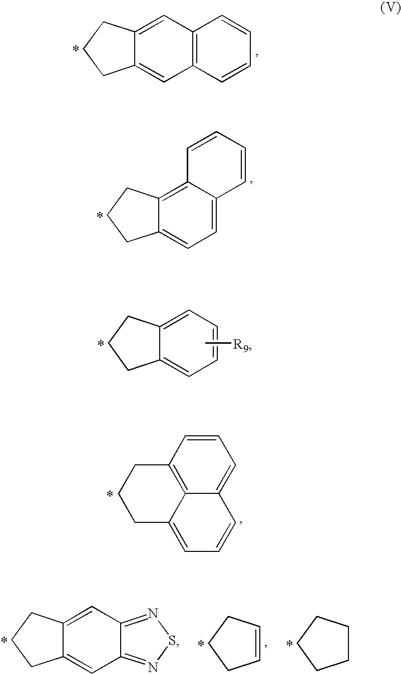
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group, and
wherein the compound is not a ubiquitin hydrolase.

14. A method for screening for a compound that decreases the level of ubiquitylated protein, said method comprising:
(a) exposing cells or tissue that express ubiquitylated protein to a test compound, and
(b) detecting the amount of ubiquitylated protein in said cells or tissue,
wherein an decrease in the amount ubiquitylated protein in cells or tissue exposed to the compound, relative to ubiquitylated protein in cells or tissue that are not exposed to the compound, indicates that the compound decreased the amount of ubiquitylated protein.

15. A method of treating or preventing cataracts of the eye, the method comprising administering to a subject in need thereof an effective amount of a heterocyclic compound having the general Formula (I): 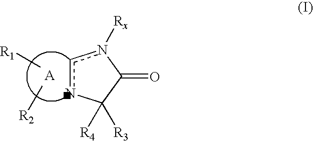
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 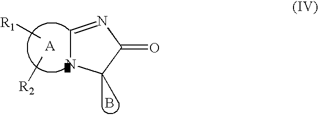
wherein B may be one or more structural units selected from structural units having the general Formula (V), 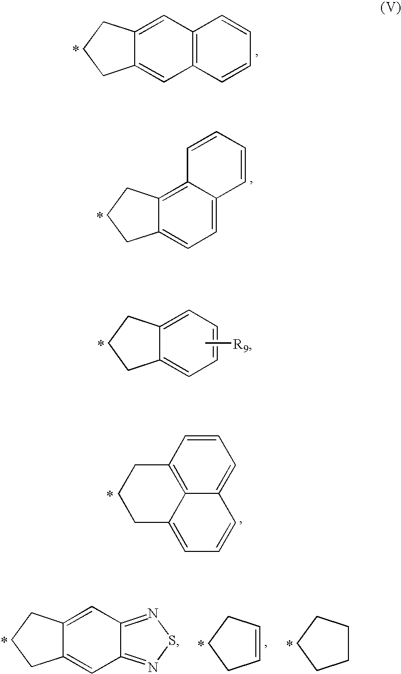
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

16. A method of treating or preventing type 2 diabetes, the method comprising administering to a subject in need thereof an effective amount of a heterocyclic compound having the general Formula (I): 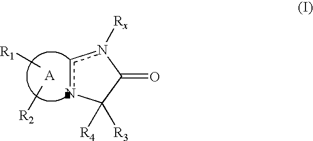
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 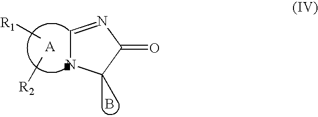
wherein B may be one or more structural units selected from structural units having the general Formula (V), 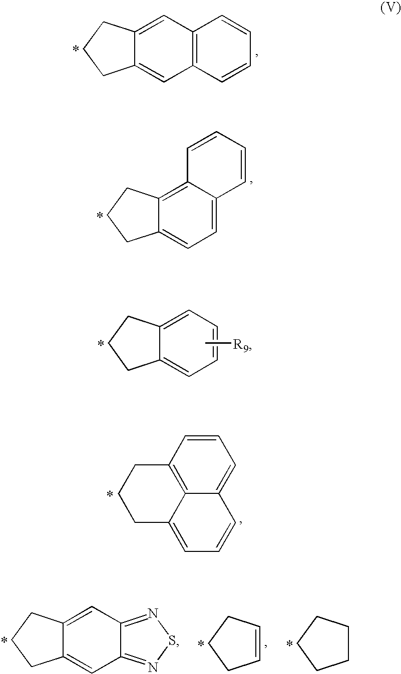
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

17. A method of treating or preventing Paget's disease, the method comprising administering to a subject in need thereof an effective amount of a heterocyclic compound having the general Formula (I): 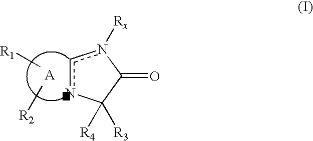
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 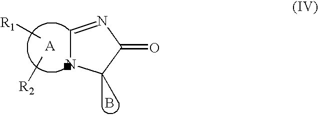
wherein B may be one or more structural units selected from structural units having the general Formula (V), 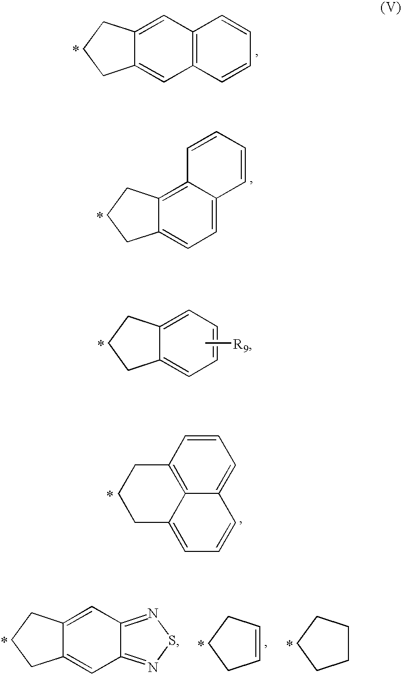
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

18. A method of treating traumatic brain injury, the method comprising administering to a subject in need thereof an effective amount of a heterocyclic compound having the general Formula (I): 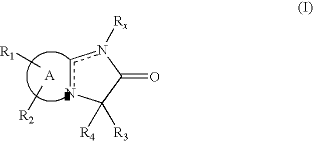
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 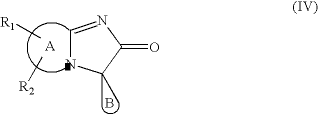
wherein B may be one or more structural units selected from structural units having the general Formula (V), 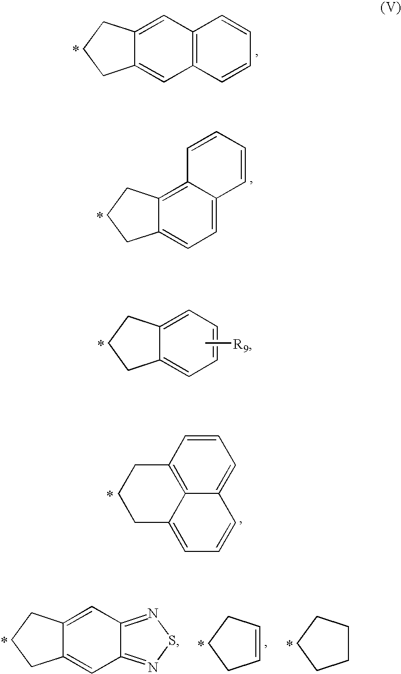
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

19. A method of treating or preventing Amyotrophic Lateral Sclerosis, the method comprising administering to a subject in need thereof an effective amount of a heterocyclic compound having the general Formula (I): 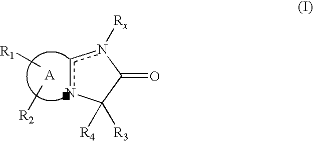
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 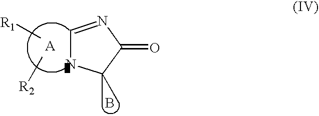
wherein B may be one or more structural units selected from structural units having the general Formula (V), 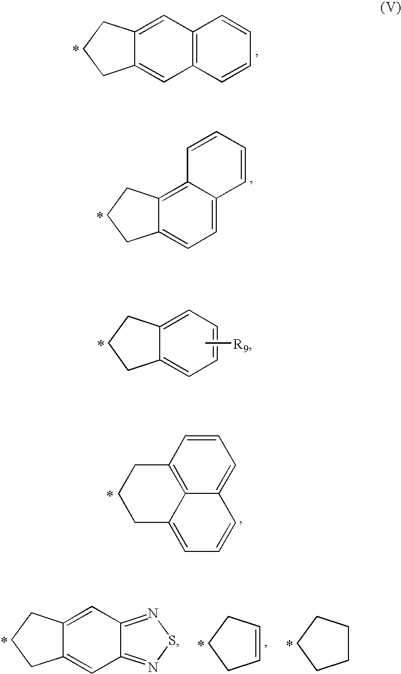
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

20. A method of treating or preventing Lewy body disease, the method comprising administering to a subject in need thereof an effective amount of a heterocyclic compound having the general Formula (I): 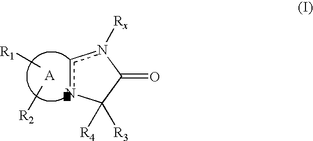
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 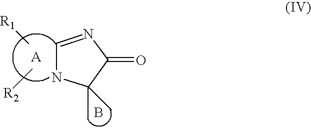
wherein B may be one or more structural units selected from structural units having the general Formula (V), 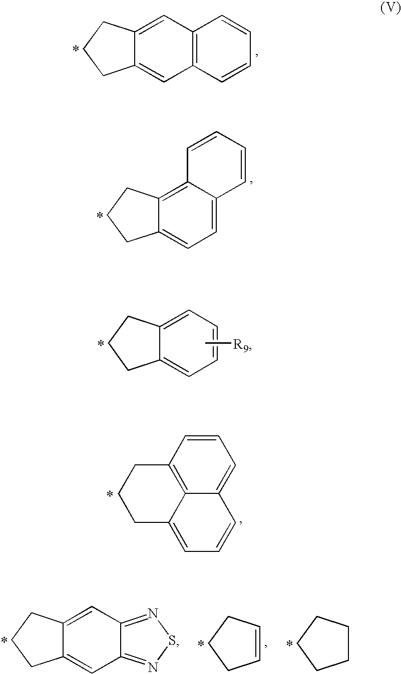
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

21. A method of treating or preventing frontotemporolobar dementia, the method comprising administering to a subject in need thereof an effective amount of a heterocyclic compound having the general Formula (I): 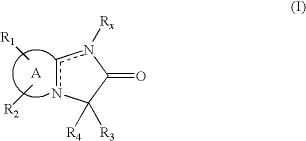
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 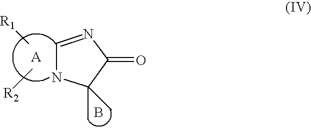
wherein B may be one or more structural units selected from structural units having the general Formula (V), 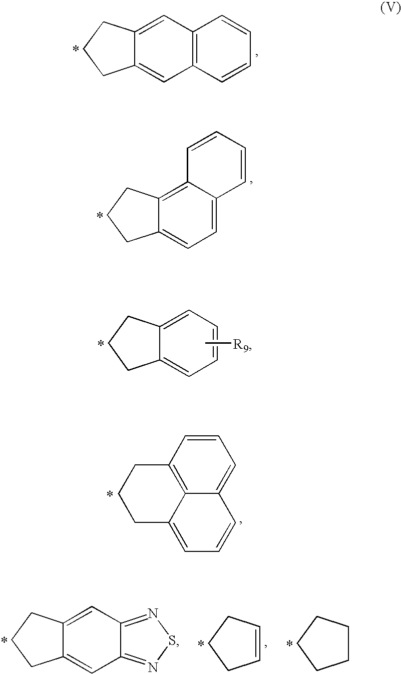
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

22. A method of treating or preventing sporadic inclusion body myositis, the method comprising administering to a subject in need thereof an effective amount of a heterocyclic compound having the general Formula (I): 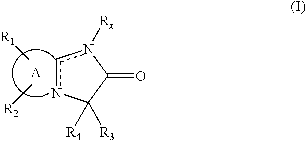
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group
consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 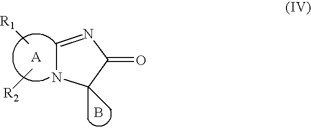
wherein B may be one or more structural units selected from structural units having the general Formula (V), 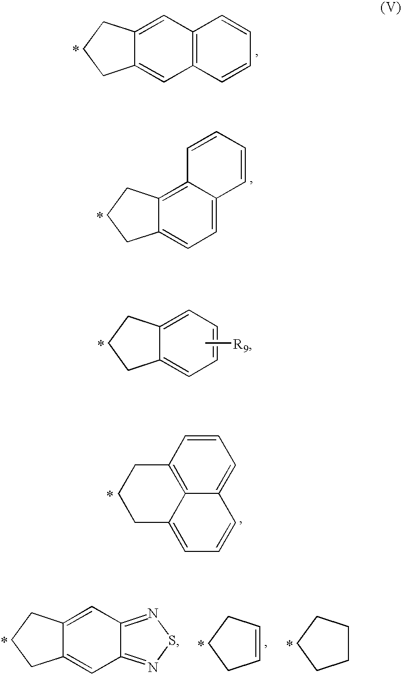
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

23. A method of treating or preventing a prion disease, the method comprising administering to a subject in need thereof an effective amount of a heterocyclic compound having the general Formula (I): 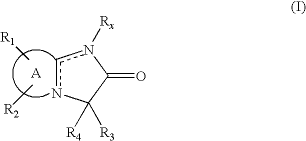
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 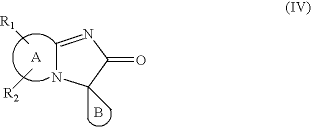
wherein B may be one or more structural units selected from structural units having the general Formula (V), 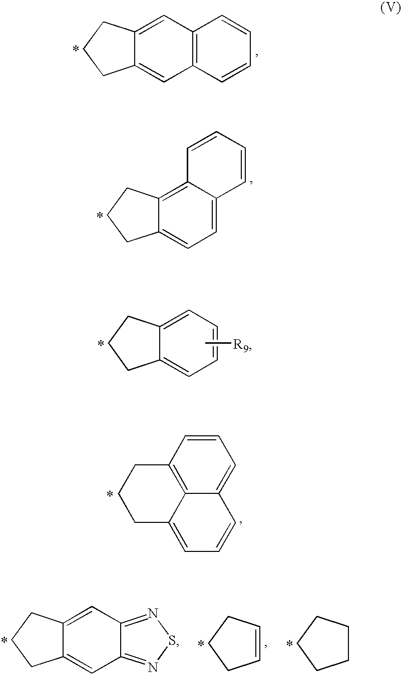
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

24. A method of treating or preventing Parkinson's disease, the method comprising administering to a subject in need thereof an effective amount of a heterocyclic compound having the general Formula (I): 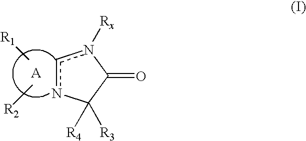
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 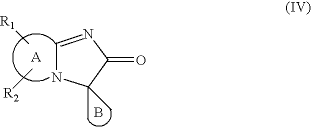
wherein B may be one or more structural units selected from structural units having the general Formula (V) 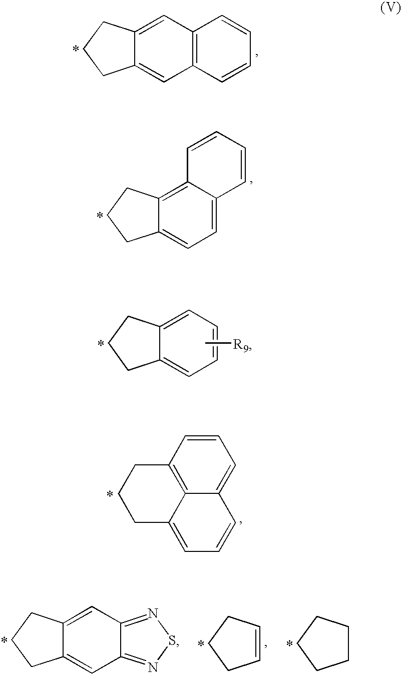
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

25. A method of treating or preventing a polyglutamine disease, the method comprising administering to a subject in need thereof an effective amount of a heterocyclic compound having the general Formula (I): 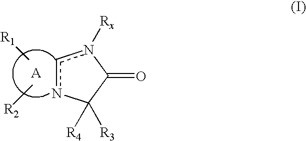
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 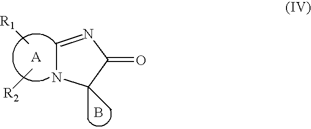
wherein B may be one or more structural units selected from structural units having the general Formula (V) 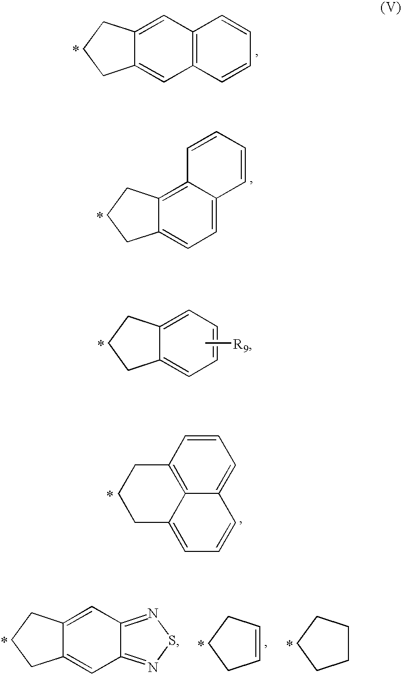
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

26. A method of treating or preventing Huntington's disease, the method comprising administering to a subject in need thereof an effective amount of a heterocyclic compound having the general Formula (I): 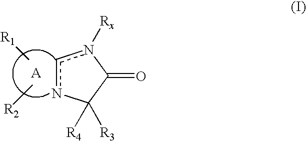
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 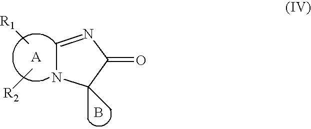
wherein B may be one or more structural units selected from structural units having the general Formula (V) 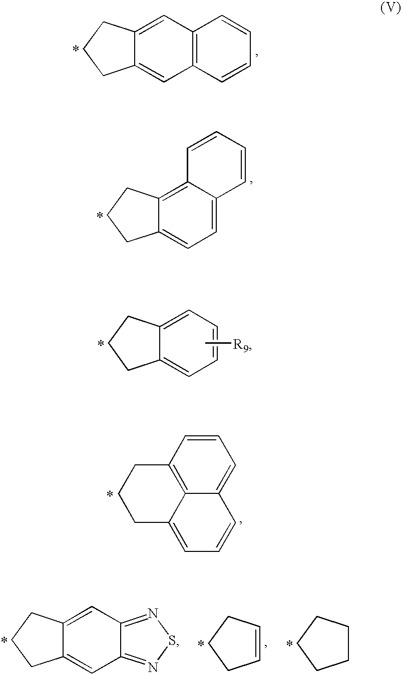
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.

27. A method of treating or preventing systemic amyloidosis, the method comprising administering to a subject in need thereof an effective amount of a heterocyclic compound having the general Formula (I): 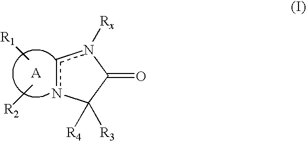
or a pharmaceutically acceptable salt, hydrate or prodrug thereof to a human subject in need thereof,
wherein Rₓ is methyl or nil;
R₁ and R₂ each are one or more functional groups independently selected from the group consisting of a hydrogen atom, halogen atom, hydroxy group, amino group, acetylamino group, benzylamino group, trifluoromethyl group, C₁-C₆ alkyl group, C₁-C₆ alkoxy group, C₂-C₆ alkenyl, C₃-C₈ cycloalkyl, benzyloxy, CH₂—R₅, and —O—(CH₂)ₙ—R₆;
R₃ and R₄ are either
(i) each one or more functional groups independently selected from the group consisting of a hydrogen atom, C₁-C₆ alkyl group, C₂-C₆ alkenyl, C₃-C₈ cycloalkyl group, CH₂—R₅, and —CH(R₈)—R₇; or
(ii) R₃ and R₄ together form a spiro ring of Formula (IV): 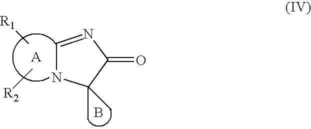
wherein B may be one or more structural units selected from structural units having the general Formula (V) 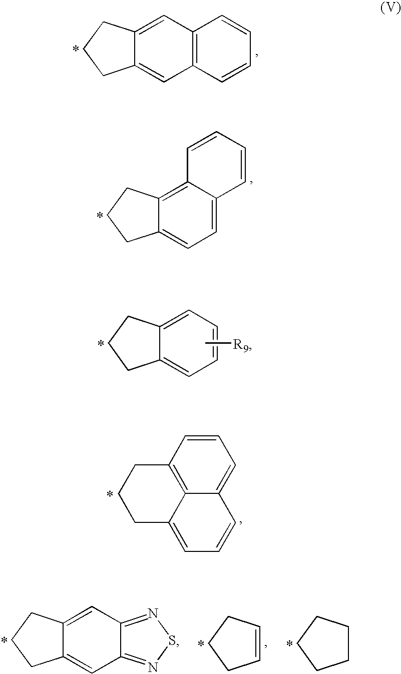
the structural unit B binds at a position marked by * in the Formula (V) to form a spiro ring; and
R₅ is naphthyl; thienyl; or phenyl, which may be substituted with C₁-C₆ alkyl, halogen atom or cyano;
R₆ is a vinyl group, C₃-C₈ cycloalkyl group, or phenyl group, and n is 0 or 1;
R₇ is one or more functional groups selected from the group consisting of a vinyl group; ethynyl group; phenyl optionally substituted by a C₁-C₆ alkyl group, C₁-C₆ alkoxy group, hydroxy group, 1 or 2 halogen atoms, di C₁-C₆ alkylamino group, cyano group, nitro group, carboxy group, or phenyl group; phenethyl group; pyridyl group; thienyl group; and furyl group;
R₈ is a hydrogen atom or C₁-C₆ alkyl group; and
R₉ is one or more functional groups selected from the group consisting of a hydrogen atom, halogen atom, hydroxy group, C₁-C₆ alkoxy group, cyano group, and trifluoromethyl group.
